SEQUENCE LISTING (86 sequences)

SEQ ID NO: 1 - DNA, 1476 bp, synthetic construct
  source 1..1476: mol_type = other DNA
atggatttcttaaccccctgtctcgtggtagcatccattgcctggatttgtatgttgatactaagagcaagaaaaccgtctaaacttcctcctggcccatatggtccgcccatcataggtaatatactacatttaggtcctaaacctcatagatctttggccgacttggcccgaaaatatgggccagtcatgaagcttaggttgggttcggtaaccacagttgttatatcttccccagaagcagctaaagctgttttacaaaaacacgattcatcattttggaatagacctgctccttcttctgttagggcggttggtcacgatgaattctcagtcgcttggctgcctgtagacaaacaatggagaaaacttcggaaaataatgaaagaattaatgttctcatcgccaagattagatgccggccaaggtatgagacgtgcaaaacttcaacaattaagtgattatgtctggggtaggtgccaggctggtagagcggtcgaggttggagaggctgcattcacgacttctctgaatttaatgtcagctacattattttctactgattttgctcgtttcgattccgatagcagtcaggaaatgaaggaagtggtatggggcgttatgaagtgtgtgggttctccaaacttggtagattactttccagtcttgaaatccctcgatccgcaagggatcctaaaagacgcaaagttctgctttgggaagttgttcgcgatctttgacgaaattctggatgaacgcttgaagatctctcgtggtgaaaagcaggatttggtagaggcactaatcgatttaaatcaacgtgacgttcctcaactaagccgcgacgatattaaccacctattgttagacttatttgttgctggttcagatactactagtggtacagtcgaatgggcaatgacagaattgattaggcatcccgaaaagatgactaagttgagaaatgagattacttcatttgttgaagagaacggcccaattgaagaaagtgatatcagcagattgccatacttacaagccgtcgttaaagagacgtttagacttcatcctgtggccccattcctacttccacataaggctagctcggacattgaaattaacggttacacggttcctaaaaatgcacagatcctggttaacatttgggcctccggaagggatccgattaactgggtggatgctgataagtttgtgccagaaaggtttttaagtgagaataaaggactgaactttataggacaggattttgaactcataccctttggcgctggtagaagaatttgtccagggttgcccttggctaatagaatggtccatcaaatgctggtgaccttcgttggaaatttcgagtggaagcttgaaggaataaaggtagaggaaatggacatggacgaaaattttggcttaaccttgcaaaaagcaatcccactacgagcgattccaacaaaattataa

SEQ ID NO: 2 - protein, 491 aa, Origanum vulgare
MDFLTPCLVVASIAWICMLILRARKPSKLPPGPYGPPIIGNILHLGPKPHRSLADLARKYGPVMKLRLGSVTTVVISSPEAAKAVLQKHDSSFWNRPAPSSVRAVGHDEFSVAWLPVDKQWRKLRKIMKELMFSSPRLDAGQGMRRAKLQQLSDYVWGRCQAGRAVEVGEAAFTTSLNLMSATLFSTDFARFDSDSSQEMKEVVWGVMKCVGSPNLVDYFPVLKSLDPQGILKDAKFCFGKLFAIFDEILDERLKISRGEKQDLVEALIDLNQRDVPQLSRDDINHLLLDLFVAGSDTTSGTVEWAMTELIRHPEKMTKLRNEITSFVEENGPIEESDISRLPYLQAVVKETFRLHPVAPFLLPHKASSDIEINGYTVPKNAQILVNIWASGRDPINWVDADKFVPERFLSENKGLNFIGQDFELIPFGAGRRICPGLPLANRMVHQMLVTFVGNFEWKLEGIKVEEMDMDENFGLTLQKAIPLRAIPTKL

SEQ ID NO: 3 - DNA, 1497 bp, synthetic construct
  source 1..1497: mol_type = other DNA
atggagtggttttgggcggctctctcattgatagtctttttgtcgttgttacaccaattactaaaaaaggagaagaaaagggaagctaacctcccaccgagtcctattgctcttcctgtaatcggccatttacacctattgggaaaaaaccttcccttaaagctgcacgcaattgccgaacgtcatggtcctatagtttttttaagattaggccttgtacgtgccttggtggttagcacagcagctggggcggagttagtgttaaaaactcatgatttggtttttagtggaagagttcatcatcaagcatctcgctaccttggttatgaccaaaagaatatcgtatttgcaccttatggggcatattggcgaaacatgcgtagattatgtatggtcaaattgttgaatgctgccaagataaatgaatttaggcccgtaagaagagctgaattagaagaaaccgttgcttccatgagaagggcagctgaagaaaggggagtggtggacgtgtcagcagtaatttccggtgtcattggagatatgaactcgttaatggttttcggaagaaaatatgtggaccgggatctggatgaagagttaggtttcaaggcagtcatagatgaaatgttgcacgttggtgccttgcctaatctaggtgacttttttccattcatggctgcactagacttgcaaggtttggatagaaggatgaaagaattgagcaagatttttgacgggttcctagaacgtataattgacgatcacttgctgaaaaagactgagaataccaaaaagggggatttcgtggatacaatgctggctgttatggaggccggcgaagccgattttgaattcgatcgtagacatgttaaagctgttctgctcgatatgcttatcgctggtatggatacgtctgccagcacagttgaatgggctctgtctgagttgattagacaccctgaaatcactaagaaactacaaaaagaactcgagcaggttgttggtatggaccagatggtagatgagtctcatctagacaaactagattacctggattccgtccttaaagagaccttaagattatacccacccggtagattgcttgtccatgaaacgatggaagaatgtacagttaacggttttcatattccaaagggtacatggactttcgtcaatatgtggagtataggaagagatccagcgatgtggcatgaaccagaaaagtttgtacctgaacgctttgccggtgaaaatttagattttctaggccaaaatttcaaattcattccattcggtgcgggccgaaggtcatgcccgggtttacagttgggcttaacttttgttaggttggtgttggcacaacttgtacattgttttgactgggaacttccaaacggaatggttccgtcagacttggatatgaacgagaaattcggtatcgtcacttctagagataagcatttaatggctattccaacctacagattaaataaataa

SEQ ID NO: 4 - protein, 498 aa, Origanum vulgare
MEWFWAALSLIVFLSLLHQLLKKEKKREANLPPSPIALPVIGHLHLLGKNLPLKLHAIAERHGPIVFLRLGLVRALVVSTAAGAELVLKTHDLVFSGRVHHQASRYLGYDQKNIVFAPYGAYWRNMRRLCMVKLLNAAKINEFRPVRRAELEETVASMRRAAEERGVVDVSAVISGVIGDMNSLMVFGRKYVDRDLDEELGFKAVIDEMLHVGALPNLGDFFPFMAALDLQGLDRRMKELSKIFDGFLERIIDDHLLKKTENTKKGDFVDTMLAVMEAGEADFEFDRRHVKAVLLDMLIAGMDTSASTVEWALSELIRHPEITKKLQKELEQVVGMDQMVDESHLDKLDYLDSVLKETLRLYPPGRLLVHETMEECTVNGFHIPKGTWTFVNMWSIGRDPAMWHEPEKFVPERFAGENLDFLGQNFKFIPFGAGRRSCPGLQLGLTFVRLVLAQLVHCFDWELPNGMVPSDLDMNEKFGIVTSRDKHLMAIPTYRLNK

SEQ ID NO: 5 - DNA, 2079 bp, synthetic construct
  source 1..2079: mol_type = other DNA
atgacctccgcactctacgcatccgatttgtttaagcagttgaaatctataatgggaaccgactccttgagcgatgacgtggttttagtgattgcgactacttcattggccttagtagccggattcgttgtacttttgtggaaaaaaacaactgctgatcgctctggtgaattaaaaccactaatgatcccaaagagtctgatggctaaagatgaagatgatgatctagacctaggttcaggtaaaacaagagtctctatattttttggcacccagacaggtacggctgaaggctttgctaaagcattgtcggaagaaattaaggccagatatgaaaaggcagcagtcaaggttatagacctggacgattacgctgcggacgacgaccaatacgaggaaaaactaaagaaggagacgttggcttttttttgtgtagctacatacggcgatggagaaccgacagacaatgctgctagattctacaagtggttcactgaagagaacgaacgtgacataaaattacagcaacttgcatacggggtgtttgcccttggcaaccgacaatacgaacactttaataagatcgggatcgttctagatgaggagctgtgcaaaaaaggagcaaaaaggcttattgaggtgggtctcggcgatgacgatcaaagcattgaggatgatttcaacgcgtggaaagaatcgttgtggagtgaattggataaacttttaaaggacgaagatgataagagtgtggctacaccatatactgccgttattcccgagtatagagtcgtaacacacgatccacgtttcacaactcaaaagtcaatggaatcaaatgtcgccaatggtaacactactattgatattcaccacccttgcagagttgatgtagcagtacaaaaagagcttcatacccatgaatctgataggagctgtatacatttggagtttgatatcagcagaacggggataacttatgagacaggagatcatgttggtgtctatgctgagaatcatgtcgagattgttgaagaagctggtaaactactggggcactccttagatttagttttctcgattcatgctgataaagaagatggctcacctttggaaagtgctgtaccaccaccatttcctgggccttgtactttaggaaccggtctggctaggtatgcagatttattgaatccgccacgaaaatcagcactagtagctttagctgcatatgccactgaacctagtgaagcagaaaaattaaaacatctcaccagtcctgatggtaaagacgaatactcacagtggattgttgcatcacaaagatcattattagaagtgatggcagcgtttccctctgccaaacctccacttggagtctttttcgctgccattgccccgagattgcaaccaagatattacagtatttcttcttctccaaggttggcacccagcagagttcatgtcacttcagcgttggtttatggtccaaccccaacaggtaggatccataaaggtgtttgttccacttggatgaaaaatgcagtcccagctgaaaagtctcacgaatgttctggtgcccctatctttataagagcttctaattttaagttgccctcgaacccgtctacacctatagttatggtgggtcccggaaccggtctagcacctttccgtggttttttgcaggaaagaatggcgctgaaagaagacggtgaagaattaggctccagtttactgttctttggctgtcggaacaggcaaatggatttcatttacgaagacgagcttaacaatttcgtcgatcagggcgtgatttccgaattaattatggccttttcgagagagggagcacaaaaggaatatgttcagcataaaatgatggagaaggctgcgcaagtttgggacttgatcaaggaggaaggatatctatatgtttgcggtgatgccaaaggtatggctagagatgttcatcgtacattacatacgatagtgcaagaacaagaaggtgtatcttcatccgaagctgaagccatcgttaagaagttacaaacggaaggtcgctatctcagagacgtatggtaa

SEQ ID NO: 6 - protein, 692 aa, Arabidopsis thaliana [sequence omitted]

SEQ ID NO: 7 - DNA, 1485 bp, synthetic construct
  source 1..1485: mol_type = other DNA
atggcctggtattggctggctttaagctttgttattttgttcctatctttcttacaacacttacttaggaagaccgatttgccaccaggaccgatagccttaccactcattggacatttgcaccttcttggcaagaacctctctagagatttacataggttagctgcgaaacatggccctattatcttcttaagactgggtactattccggcagttgtagtttcttcggctgctggtgctgaactagttttaaagacacatgatctggcattcgcaggacgaactcatcatcaagctgcaaggtacattggttacgaccaacgtaacattttgtttgcaccttatggcgcctactggcgcaacatgaggagattgtgcacggtagaactgctatcatcggccaaaatcaatgaattccgaccaataagaagagccggcatgggggaaatggttggttcattaagacgtgatgcagaaagaggtgaaatcgtcgatttaacagcaagggtaagtggagttttgggggatatgaattgtttaatggtgtttggtcgaaaatacgtcgatcgtgatttggatgaaaagttgggcttcaaagccgtgattgatgaaacactgcaagttgctgctatcccgaatttgggggatttttttccattcatcgccgcactagacgtgcagggactcgacaggcggatgaagaagctatcaagcatattcgatgcttttcttgacaagattatcgacgatcatttgcaaagaaagctagagaaaactcacaatcacgattttgtcgacactatgttggcggttatggattcaggtgaagctggttttgagtttgatagaagacatgtcaaagcggtattattggacattttaattggcgggatggatacaacactatccactgtggaatgggctatatcagagctgattagacaccccaaagtcaccaagaaacttcagaacgagcttgaaagaatagtttcccttgaccagatggtggacgagtctcatttagataaattggaatatttagacttggtaattaaggagaccttacgcctccacccacccggtaggttgttggtgcatgagagtatggaaaattgcacggtcaacggttttcatattccaaaaggtacatggacctttgtaaatatatggacgatcggtagagatccaaatttctggcatgaacctgacgaatttgttcctgaacgttttgtaggagaaaatgttgacctgttgggtcaacacttccaattaatcccttttggttctggtagaagatcctgtcctggacaacaattaggtctaaccttggttaaattggttgttgctcagcttgtgcactgtttcgattgggaattgcctgatggagtcgagcccagagatcttgatatgactgaacattttggcatagtcactagtagagacaaacatttaatggcaataccatcttatcgtctatataaataa

SEQ ID NO: 8 - protein, 494 aa, Salvia hispanica
MAWYWLALSFVILFLSFLQHLLRKTDLPPGPIALPLIGHLHLLGKNLSRDLHRLAAKHGPIIFLRLGTIPAVVVSSAAGAELVLKTHDLAFAGRTHHQAARYIGYDQRNILFAPYGAYWRNMRRLCTVELLSSAKINEFRPIRRAGMGEMVGSLRRDAERGEIVDLTARVSGVLGDMNCLMVFGRKYVDRDLDEKLGFKAVIDETLQVAAIPNLGDFFPFIAALDVQGLDRRMKKLSSIFDAFLDKIIDDHLQRKLEKTHNHDFVDTMLAVMDSGEAGFEFDRRHVKAVLLDILIGGMDTTLSTVEWAISELIRHPKVTKKLQNELERIVSLDQMVDESHLDKLEYLDLVIKETLRLHPPGRLLVHESMENCTVNGFHIPKGTWTFVNIWTIGRDPNFWHEPDEFVPERFVGENVDLLGQHFQLIPFGSGRRSCPGQQLGLTLVKLVVAQLVHCFDWELPDGVEPRDLDMTEHFGIVTSRDKHLMAIPSYRLYK

SEQ ID NO: 9 - DNA, 1497 bp, synthetic construct
  source 1..1497: mol_type = other DNA
atgaattttaatatcttaattgattttttaacttcaagcctggtggccgcttccatcgcatggatttgtattctcatttctcgcgctaggaagagttcaaaattgccgcctggcccttacggtcatccggtaattggaaatgttttacatttaggacctaagccacatcggtcgctcgccaaactgagtcaaaaatatggaccagttatgtccttaaaattggggagtatcaccactgtggtcattagttcagcagaaactgcaaagttggttttgcagaaacacgatagcagcttctccaatcgaactattccatctgcatccaaagcattaaggcatcatgatttttcagtagtatggttgcccgtcggtagccaatggaggaaattaagacgaatttgcagagaacaaatgttctctgccctcagattagacgcctctcaaggtttaagaagggaaaaattgcaaaagttaagagatttcgtagctgaaagatgtgatagaggtgctgccgtcgatgttggcgatgctgcttttaccacggcactaaatttaatgagtgcctctttgttttcggctgaattcgctcgctttgattctgaaagttcccaagaaatgaaggaagttgtgtggggcgtgatgaaaagtgtagggtcaccaaacttcgcagattattttccattgctgaagccagctgacccccaaggcatatttcaagccgcggaattcaacttcggtaagctattcgcgaaactagatgaaataatcgatgagaagcttaagtcaggtggcgaaaagcgtgacctagttgaagctctattagaaataaatagacgtgacgaagatcaactatcgagagaagatatcagacatttgctattggatctgtttgttgcaggtacagacactacatcgggtacggttgaatgggccatgactgagttgattaggaatcctcagaaacttgaaaaacttcgtaacgaaataagatctttcatgtcagagcacggaaagcaacaggtccaggagtctgatatctcctcattaccatacttacaagcagtcgttaaggaaacctttagacttcatccaacagcgccttttttactaccccacaaggcttcttctgacgttgaaataaatggatatattgtccctgaaaacgcgcagatacttataaatgtctgggcttcaggtcgtgattctaatatctggaaaaaccctaacgagttcttgcccgagagatttctgaatgagaatgggaacgtggactttaaaggaagagattttgagttgattccttttggtgcaggtaggaggatatgtccaggtttgccacttgcagacagaatggttcaccttatgttggtaacattcgtgggtaactacgaatggaaattggaaaacattaaaccggaggaaatggacatgaatgagaattttgggatcaccctgcaaaaagctgttcctttgaaagctattccaacaaaattataa

SEQ ID NO: 10 - protein, 498 aa, Salvia splendens
MNFNILIDFLTSSLVAASIAWICILISRARKSSKLPPGPYGHPVIGNVLHLGPKPHRSLAKLSQKYGPVMSLKLGSITTVVISSAETAKLVLQKHDSSFSNRTIPSASKALRHHDFSVVWLPVGSQWRKLRRICREQMFSALRLDASQGLRREKLQKLRDFVAERCDRGAAVDVGDAAFTTALNLMSASLFSAEFARFDSESSQEMKEVVWGVMKSVGSPNFADYFPLLKPADPQGIFQAAEFNFGKLFAKLDEIIDEKLKSGGEKRDLVEALLEINRRDEDQLSREDIRHLLLDLFVAGTDTTSGTVEWAMTELIRNPQKLEKLRNEIRSFMSEHGKQQVQESDISSLPYLQAVVKETFRLHPTAPFLLPHKASSDVEINGYIVPENAQILINVWASGRDSNIWKNPNEFLPERFLNENGNVDFKGRDFELIPFGAGRRICPGLPLADRMVHLMLVTFVGNYEWKLENIKPEEMDMNENFGITLQKAVPLKAIPTKL

SEQ ID NO: 11 - DNA, 1482 bp, synthetic construct
  source 1..1482: mol_type = other DNA
atggcttggttttggttagcactctcctttattattttattatctttcctgcagcacttgttgcggcgtagagatttgcctcctggccccatcgccttaccacttatcggacacctacacttattgggtaaaaatctctcgagagatttacatagacttgctgcaaagcatggaccaattatttttttacgtctagggacaattccagcagttgttgtttcatcagccgctggtgcggaattagtgctcaaaacacatgacctagcgttcgcgggaagaacacatcatcaggcagctagatatattggttacgatcaacgtaatatactgttcgctccgtatggtgcatattggcgaaatatgagaaggttgtgtacagtggaactgctatcttctgcaaaaataaatgagtttcgaccaattaggagagctggaatgggtgaaatggttgggtctttgcgaagagatgccgaaagaggcgaaatagtagatttgactgctagagtatctggggttttgggtgacatgaactgcttgatggtattcggaagaaagtatgtagatagagatttagatgagaaactgggttttaaagctgtcattgacgaaacactacaggtcgcagccatacctaatctcggcgacttttttcctttcattgctgctttggacctacagggccttgacaggcgtatgaaaaagctgtcctctatcttcgatgcctttttggacaagattatcgacgaccacctacaaaggaaaccagaaaagacgcataaccgcgattttgtggatacaatgttagctgtgatggatagtggtgaagcaggtttcgaattcgataggagacacgtgaaggcagttttgctggatatactaataggcggtatggatactactttgagtaccgttgagtgggccatcagcgagcttatccgccatccaaaggttaccaagaaattacaatccgagttggaaaggatcgtgtcattggacgaaatggtagacgagtcacaccttgataaacttgagtacttagacttagtcgttaaagaaactttacggctgcacccccccggaagacttttagtccacgaatccattgagaattgcactgttaacggcttccatattccgaaaggtacctggacgtttgtcaatatctggactattggtagagatccaaatttttggcatgaacctgatgaattcgtcccagaaaggcttgttgggaagaacgtcgatttactaggccaacattttcaacttattccttttggaagcggtaggagaagttgtcctggacaacaattgggtttgacgttagtaaagttggttgttgctcaattggtacattgttttgattgggaactaccagatggtgtggaaccgcgtgatttggatatgaccgaacattttggtatagttactagtagagccaaacatttaaactcaataccatcgtacagattatacaaataa

SEQ ID NO: 12 - protein, 493 aa, Salvia splendens
MAWFWLALSFIILLSFLQHLLRRRDLPPGPIALPLIGHLHLLGKNLSRDLHRLAAKHGPIIFLRLGTIPAVVVSSAAGAELVLKTHDLAFAGRTHHQAARYIGYDQRNILFAPYGAYWRNMRRLCTVELLSSAKINEFRPIRRAGMGEMVGSLRRDAERGEIVDLTARVSGVLGDMNCLMVFGRKYVDRDLDEKLGFKAVIDETLQVAAIPNLGDFFPFIAALDLQGLDRRMKKLSSIFDAFLDKIIDDHLQRKPEKTHNRDFVDTMLAVMDSGEAGFEFDRRHVKAVLLDILIGGMDTTLSTVEWAISELIRHPKVTKKLQSELERIVSLDEMVDESHLDKLEYLDLVVKETLRLHPPGRLLVHESIENCTVNGFHIPKGTWTFVNIWTIGRDPNFWHEPDEFVPERLVGKNVDLLGQHFQLIPFGSGRRSCPGQQLGLTLVKLVVAQLVHCFDWELPDGVEPRDLDMTEHFGIVTSRAKHLNSIPSYRLYK

SEQ ID NO: 13 - DNA, 1491 bp, synthetic construct
  source 1..1491: mol_type = other DNA
atggatcttttaacgtccttgatattcgtagcttccatagcttggatctgcattttgattctaacatctaattcaagagcaaggaaatcatcaaaactcccacctggtccatatgggccccctattataggtaatattttacatctaggtcccaagccacataggtcgttggctaagttatcccaaaagtatggcccggtgatgtccctgaaactgggtagcataactacggttgtgataagttcagctgaaacagctaaactcgtacttcaaaagcatgattcaagcttcagtaacaggactattccttctgcttctaaggctttgagacatcatgaattttcggttgcatggttaccagttggttctcaatggagaaagttgagaaagatctgtagagagcaaatgtttagcgcgccacgtttagatagttctgaaggattgaggagagaaaaattgcataaattacgtgatttcgtcgccgagagatgtgatagaggtgtcgccgtcaacgttggcgacgcagcatttacaaccgcactaaacttgatgtccgcgtccttattttcagttgaattcgccaggtttgactcagaatcttctcaggaaatgaaagaggtagtttggggtgtaatgaaatctgttggtagtccgaatctggccgattactttcctctcctaaaaccagcagacccgcagagaatttttaaagcagcggaattcaactacggcaaattatttgcaaagctagatgaaatcatcgatgaaaaacttaaatctggtggcgacaaaagggatttagtggaggcattactagagattaataggagagatgaagcccaactttcaagagacgacattagacacttgcttcttgatttatttgttgctggtactgacactacctcaggtactgtagaatgggcaatgacagaattaattcgtaatccacagaaaatggccaagttgagatccgagattagatcttttatgagtgagaacggaaagcaacaagtagaagagagtgacatcactagcctaccatacttgcaagctgtggttaaagaaaatttccggctacaccctgccgctccgcttttggtccctcacaaagctaattgcgacgttgaaatcaatggatatattgtgccagaaaacgctcaaatattgattaacgtctgggcgtctggccgcgattcgaacatttggaagaatcctaatgaatttctgccagaacgattcttgaatgaaaacggttcggttgatttcaagggacgtgattttgagttgataccatttggggctggacgaagaatatgtcccggattacctttagctgatcgcatggtgcaccagatgctggtcacgttcgtcggtaattatgagtggaagttagaaaataccaagcccgaagaaatggatatgaatgaaaactttgggatcaccctgcaaaaagcagttccattaaaagccatccctacaaaattgtaa

SEQ ID NO: 14 - protein, 496 aa, Salvia splendens
MDLLTSLIFVASIAWICILILTSNSRARKSSKLPPGPYGPPIIGNILHLGPKPHRSLAKLSQKYGPVMSLKLGSITTVVISSAETAKLVLQKHDSSFSNRTIPSASKALRHHEFSVAWLPVGSQWRKLRKICREQMFSAPRLDSSEGLRREKLHKLRDFVAERCDRGVAVNVGDAAFTTALNLMSASLFSVEFARFDSESSQEMKEVVWGVMKSVGSPNLADYFPLLKPADPQRIFKAAEFNYGKLFAKLDEIIDEKLKSGGDKRDLVEALLEINRRDEAQLSRDDIRHLLLDLFVAGTDTTSGTVEWAMTELIRNPQKMAKLRSEIRSFMSENGKQQVEESDITSLPYLQAVVKENFRLHPAAPLLVPHKANCDVEINGYIVPENAQILINVWASGRDSNIWKNPNEFLPERFLNENGSVDFKGRDFELIPFGAGRRICPGLPLADRMVHQMLVTFVGNYEWKLENTKPEEMDMNENFGITLQKAVPLKAIPTKL

SEQ ID NO: 15 - DNA, 1491 bp, synthetic construct
  source 1..1491: mol_type = other DNA
atggattttcttacgtcgagtattttcgccgcgagtattgcttggatttgcatcttaatcctgacgagtaatagccaagctcgtaaattgtcaaagttaccacctggtccctatggtctgcctataattggtaacatcttgcagcttggtccaaagcctcaccattcattagcaaaactttctcaaaagtacggcccggtaatgttgttaaagctaggctccattaccacagtggttatttcgtccgcagaaaccgcaaagttggttcttcaaaagcacgactcaagtttttccaatcgtacaattccttctgctataacagctttgcgtcaccatgaattttccgtaggttggttgccagtaggtaatcaatggaggaaattgcgtaagatatgtaaagaacagatgttctctgccccaaggctagatgctagtgaaggattaagaagggaaaaattgcaaaaattgagagactttgttgcagaaagatgtgatagaggtacagctgtcgatgttggagaggctgcttttactacttcattgaatctcatttctgctagcttattttccatggagtttgcccgattcgattcggaatcttctcaggaaatgaaagaagtcgtttggggagtgatgaaatctatcgggtcacctaatttagccgattatttcccgctgctaaaaccagcagatcctcaaggcatcttgaaggccgcagagtttaatttcgttaaactcttcgcgaaactcgatgagataattgacgaaaagttaaaatctggagttgaaaaaagagacctagtcgaggctttgttggaaatccatcgcagagatgaggcacaattgtcaagagatgatattagacatctactacttgatcttttattagccggtacggatactacctccggtactatggaatgggcaatgactgagttaatctcaaatccccagaaaatggtcaagttaagggacgaaattagatcctttatgtctgagaacggcaaacctcagattgacgaaagtgacatttctagcctgccttatttacaagcggtggtaaaagaaactttcaggttgcacccagctgcacccttcttattaccgcataaggcaagctcgtacgtggaaattaacggatacatagttccagaaaatgcccaaattttgataaatgtttgggcttcaggtagagattctagaatttggcggaacccagatgagtttttacccgaaagatttcttaacgagaacaacaatatagattttaagggtcgcaactttgaacttatcccattttcaacaggtcgaaggatatgtccaggcttgccactagcggacagaatggtgcatcaaatgctggtcactttcgtagggaatttcgaatggaaactagagaatatgaagctggaagaaatggacatgaacgaaaattttgggatcaccctgcaaaaagctgttcctttgaaggccataccaacaaaattataa

SEQ ID NO: 16 - protein, 496 aa, Salvia hispanica
MDFLTSSIFAASIAWICILILTSNSQARKLSKLPPGPYGLPIIGNILQLGPKPHHSLAKLSQKYGPVMLLKLGSITTVVISSAETAKLVLQKHDSSFSNRTIPSAITALRHHEFSVGWLPVGNQWRKLRKICKEQMFSAPRLDASEGLRREKLQKLRDFVAERCDRGTAVDVGEAAFTTSLNLISASLFSMEFARFDSESSQEMKEVVWGVMKSIGSPNLADYFPLLKPADPQGILKAAEFNFVKLFAKLDEIIDEKLKSGVEKRDLVEALLEIHRRDEAQLSRDDIRHLLLDLLLAGTDTTSGTMEWAMTELISNPQKMVKLRDEIRSFMSENGKPQIDESDISSLPYLQAVVKETFRLHPAAPFLLPHKASSYVEINGYIVPENAQILINVWASGRDSRIWRNPDEFLPERFLNENNNIDFKGRNFELIPFSTGRRICPGLPLADRMVHQMLVTFVGNFEWKLENMKLEEMDMNENFGITLQKAVPLKAIPTKL

SEQ ID NO: 17 - DNA, 1497 bp, synthetic construct
  source 1..1497: mol_type = other DNA
atgaactttaattttttgattgactttttaaccagctccttaattgcagcttctattgcatgggtctgtattctcatttcacgtgcgcgcaaatcatctaagctgccgcccggcccttacggattgcctgttatcggcaatatcttacatttaggcccaaaaccccacagaagtttggcggagttgagccagaaatacgggcccgtgatgagcttgaagttaggtagtataactaccgttgttattacatccgccgaaactgcgaagcttgtgttacaaaagcatgattcatcattctcgaataggacattaccttctgcttcaaaagccttgagacatcatgaattcagtgtagcttggttaccagtcggttcacaatggagaaaattaagaaaaatttgcagggaacaaatgttttccgccccacgtttggatgcttctgaaggtttgagaagggaaaaattgcaaaaattgcaagatttcgttgctgaaagatctgatagaggggcagctgttaatgtaggtgatgcagcatttactacggccttaaatctcatgtcggctagtctattctctgtagaatttgcaaggtttgattccgaaagctctcaggaaatgaaagaagtggtatggggtgttatgaaagccgtaggttcacctaatttggcggattacttcccacttctcaaacctgcagatcctcagggtatctttaaagcagcagagtataattatggcaagctttttgccaaactggacgaaatcatagacgagaagctaaagagtgaaggagaaaaacgagatcttgtcgaagcattgctggaaattaatagaagggatgagactcaattgtcacgagacgatatcagacatctactattggacctgttcgtggctggtactgatacaacctctggaacggttgaatgggccatgacagagttaatacgcaatccccaaaaaatggctaagctgagaagcgaaatcaggtcctttatgagtaaaaacgggaagcaacaagtagaggagtccgacatttcttctttaccttatcttcaggctgttgtcaaagaaaactttagactacaccctgctgctccatttttagttccacacaaagccaattgtgatgttgaaataaacgactatattgtcccggaaaatgcacagattcttattaacgtgtgggcctcgggtcgtgattcgaacatctggaagaacccaaacgaatttctgccggaacgtttcttaaatgaaaatgacaacatagattttaagggtagagatttcgagcttatcccattcggagctggaagaagaatatgtccaggtttgccattggctgatcggatggttcatcaaatgttagtcacattcgtgggtaattacgaatggaagttagaaaatatgaaaccagaggagattgacatgaacgagaactttggcataaccctacaaaaagcagttcctctaaaggctataccaactaagctataa

SEQ ID NO: 18 - protein, 498 aa, Salvia hispanica
MNFNFLIDFLTSSLIAASIAWVCILISRARKSSKLPPGPYGLPVIGNILHLGPKPHRSLAELSQKYGPVMSLKLGSITTVVITSAETAKLVLQKHDSSFSNRTLPSASKALRHHEFSVAWLPVGSQWRKLRKICREQMFSAPRLDASEGLRREKLQKLQDFVAERSDRGAAVNVGDAAFTTALNLMSASLFSVEFARFDSESSQEMKEVVWGVMKAVGSPNLADYFPLLKPADPQGIFKAAEYNYGKLFAKLDEIIDEKLKSEGEKRDLVEALLEINRRDETQLSRDDIRHLLLDLFVAGTDTTSGTVEWAMTELIRNPQKMAKLRSEIRSFMSKNGKQQVEESDISSLPYLQAVVKENFRLHPAAPFLVPHKANCDVEINDYIVPENAQILINVWASGRDSNIWKNPNEFLPERFLNENDNIDFKGRDFELIPFGAGRRICPGLPLADRMVHQMLVTFVGNYEWKLENMKPEEIDMNENFGITLQKAVPLKAIPTKL

SEQ ID NO: 19 - DNA, 1479 bp, synthetic construct
  source 1..1479: mol_type = other DNA
atggatatcttgaccagctcgataatattggcatcaattgcatggacgtgcattttaatttctcgagccagaaagttgtcaaaactacccccggggccatatggcttgccaatcatcggaaatattttacatttagggccaactccacaccggtcgttagccaaattgtctcataagtacggccccgttatgtctttaaagctgggatcaattaccacagtagtcatctcctctgcggaaacagcgaagttagtccttcaaaaatacgatagtagcttttccaatagaactattccattagcttctaaagctctgcgtcatgacgaatttagtgttggttggctaccagtcggctctcaatggaggaaattaaggaagatatgtagagaacaaatgttttcggctcccagattagatgctagtgaaggtcttagaagagaaaaacttcagaaactacgtgatttcgtagctaaccgctgcgaatcaggatcagctgttgatgtgggtgaagcagctttcacaacagctttaaatttaatttccgccagcttatttagtgcggagtttgcacgtttcgattctgagtctagtcaagaaatgaaagaagccgtctgtggtgtaatgaaatccgttggatctccaaatttcgcagattatttcccattgctcaagccagccgatcctcagggtcttctcaaggccgcagaactgtcaactggtatgttgttcgttaaactcgacgaaatcattgacgaaaagctgaaatcacgtggtgagaagcgagaccttgtagaagctttgctggaaataaaccaaagggatgaggcacaattgagtagagatgacataagacacttactgctagatttgttagttgctggtacggacacatcatccgcaactgtcgaatgggcaatgaccgagctaatcagaaatccgcacaagatgactaagttgagaaacgagataaaatcatttatgtccgagaatggcaaacagcaagttcaggaaagcgacatacctctattgccttacttgcaagccgtggtaaaagaaacatttagactgcatccggcggctccatttttgttacctcataaagctagtagcgatgttgagatcaacggttatatagtgccagcaaacgcacagattttgattaatgtttgggcttcgggtagggattccagaatttggaagcatcctgacgagtttcttcctgaaagatttttgaatgaaaacgaaggggttgatttcaaaggccaagatttcgaacttattcccttttctgccggtaggagaatttgtcctggattgccactagccgatcgcatggtgcaccaaatgctagtcaccttcgtggaaaattatgaatggaagttggaaaatatgaaaccggaggaaatggacatgaacgagtcttttggtatcacgcttaggaaggctattcctttaaaagctatacctactaaattataa

SEQ ID NO: 20 - protein, 492 aa, Salvia hispanica
MDILTSSIILASIAWTCILISRARKLSKLPPGPYGLPIIGNILHLGPTPHRSLAKLSHKYGPVMSLKLGSITTVVISSAETAKLVLQKYDSSFSNRTIPLASKALRHDEFSVGWLPVGSQWRKLRKICREQMFSAPRLDASEGLRREKLQKLRDFVANRCESGSAVDVGEAAFTTALNLISASLFSAEFARFDSESSQEMKEAVCGVMKSVGSPNFADYFPLLKPADPQGLLKAAELSTGMLFVKLDEIIDEKLKSRGEKRDLVEALLEINQRDEAQLSRDDIRHLLLDLLVAGTDTSSATVEWAMTELIRNPHKMTKLRNEIKSFMSENGKQQVQESDIPLLPYLQAVVKETFRLHPAAPFLLPHKASSDVEINGYIVPANAQILINVWASGRDSRIWKHPDEFLPERFLNENEGVDFKGQDFELIPFSAGRRICPGLPLADRMVHQMLVTFVENYEWKLENMKPEEMDMNESFGITLRKAIPLKAIPTKL

SEQ ID NO: 21 - DNA, 1086 bp, synthetic construct
  source 1..1086: mol_type = other DNA
atgatttgcaaagaacaaatgtttagcgtttctcgtttagacgcatcacaatccctacgacgcgaaaaattgcaaaagctttgtgattatgttaaggaatgttcagaaacagggagggctgtagatattggtgaggccgcttttacgacaagtcttaatctgatgtctgcgacacttttctccaaagaattcgcacaatttaactccgattcaagtcaggagatgaaggacgtagtatggggtgtgatggaatgtgtgggcagcccaaactttgctgattactttccagttttaaagagtatagatccacaaggtatctctaagcagctggaaattcattttgagaaattgttcgcgatattcgacggtataatcgatgataaaatgaaatcgcgtgccgagaataacgatttggtggaagccttgattgatatcaaccagagagacgaagctgagttgtccagggatgaaataaatcatcttctattggacctgtttgttgctggagttgataccacttctggtactgtagagtgggccatgaccgaactgcttagaaatcctgaaaagctatctagagttagaaatgaaatccaggatgtcataaaacaaaacggccaagtggacgaatcagatattcctcaattgccttatttacaagctgttgtcaaagagacctttagattacacccagcagctccgtttttagtaccacataaggctaatatggatgtcgaaatcaatggttacattgtccccaaagacacacaagtgctcgtcaatgtttgggcatcgggaagagatccaaatatttggaaaaatccagattctttcgaacctgaaagattcttaaacgacaacgtacacattgacttcaaaggtaaggattttgagctcattccctttggttcaggtaggagaatttgccctggcttaccgttggccaatcggatggtccatctaatgttagcaacgctagttgggaagttcgattggaagttggaaggaatattggatatggacgaaaagtttggattaactttgcagaaaagtatccctttaaaagcagttcccactaaattataa

SEQ ID NO: 22 - protein, 361 aa, Scutellaria montana
MICKEQMFSVSRLDASQSLRREKLQKLCDYVKECSETGRAVDIGEAAFTTSLNLMSATLFSKEFAQFNSDSSQEMKDVVWGVMECVGSPNFADYFPVLKSIDPQGISKQLEIHFEKLFAIFDGIIDDKMKSRAENNDLVEALIDINQRDEAELSRDEINHLLLDLFVAGVDTTSGTVEWAMTELLRNPEKLSRVRNEIQDVIKQNGQVDESDIPQLPYLQAVVKETFRLHPAAPFLVPHKANMDVEINGYIVPKDTQVLVNVWASGRDPNIWKNPDSFEPERFLNDNVHIDFKGKDFELIPFGSGRRICPGLPLANRMVHLMLATLVGKFDWKLEGILDMDEKFGLTLQKSIPLKAVPTKL

SEQ ID NO: 23 - DNA, 1479 bp, synthetic construct
  source 1..1479: mol_type = other DNA
atggattttttaactagctcagtgattttagcttcaattgcatggatatgcatgctaattagtcgagcgagaaaagttagcaaacttcccccaggtccatacggattacccataataggtaatatcctacagttggggcctaaaccccatcattctttagcaaaactctcacgaaagtatggacctgttatgtctcttaagttggggtccattacgacagttgtaattagtagtgcagaaacggctaagctcgtgcttcaaaaacatgacagctcattcagtaataggactgtactatctgcaataactgcactacgccaccacgaattctcggtaggatggcttccggtcggcaatcaatggaggaaattaagaaagatctgtaaagagcaaatgttttcggcaccacgcttagacgctagtgaaggtttaagaagggaaaaattacaaaaactacaggattatttagctgaaagatgtgaaagaggtacagctgttgatgtaggtgaggccgcctttacaacagctttgaatttgatgagcgcttctctattttccgccgaatttgttcaatttggctccgaaaccagccaggagatgaaagaggccatcagttcagtcgtaaagtcaataggtcgtcctaacctcgctgattattttccggttctgaaggccgttgacccccagggaatatacaaggaaacggaattgaatgtgggtcggctatttgtcaaattggatgaaatcatagatgaaaaattaagatccaggggtgagaagagagatttagtggaagtgttgcttgaaattcacaggagggacgaagcacaactttctagagacgatatcaggcatcttttgttggatttactggttgctggcaccgataccacctcaggaactatggaatgggccatgactgaactgattcgtaatccacagaagatggtcaagttgagaaatgagatcagatcattcatgtccgaatctggtaaacaacaaattgaggagtccgatatttcgtctttgtcttacttacaagctgtagttaaggaaaatttcagattgcatcctgcagcgccatttttggttccacataaggctaactgcgatgttgaaataaacggttatatcattccgaaaaatgcccaaatattcgtgaacgtctgggcatcggggagagactctaaaatctggaagaacccagatgaattcttgcctgaaagatttctgaacgaaaatgataacattgactttaaaggtagagactttgaactaatcccattcggttctggccgtcgtatgtgtccaggcttacctttagcggacagaatggtccaccaaatgttggttactttcgtcggaaattacgattggaagctggaaaacatgaaacctgaggagatggatatgaacgagaatttcggtattacactggaaaaagcgattcctttgaaagctattccaattaaagtataa

SEQ ID NO: 24 - protein, 492 aa, Salvia hispanica
MDFLTSSVILASIAWICMLISRARKVSKLPPGPYGLPIIGNILQLGPKPHHSLAKLSRKYGPVMSLKLGSITTVVISSAETAKLVLQKHDSSFSNRTVLSAITALRHHEFSVGWLPVGNQWRKLRKICKEQMFSAPRLDASEGLRREKLQKLQDYLAERCERGTAVDVGEAAFTTALNLMSASLFSAEFVQFGSETSQEMKEAISSVVKSIGRPNLADYFPVLKAVDPQGIYKETELNVGRLFVKLDEIIDEKLRSRGEKRDLVEVLLEIHRRDEAQLSRDDIRHLLLDLLVAGTDTTSGTMEWAMTELIRNPQKMVKLRNEIRSFMSESGKQQIEESDISSLSYLQAVVKENFRLHPAAPFLVPHKANCDVEINGYIIPKNAQIFVNVWASGRDSKIWKNPDEFLPERFLNENDNIDFKGRDFELIPFGSGRRMCPGLPLADRMVHQMLVTFVGNYDWKLENMKPEEMDMNENFGITLEKAIPLKAIPIKV

SEQ ID NO: 25 - DNA, 1374 bp, synthetic construct
  source 1..1374: mol_type = other DNA
atgtttcccattatcggcaacattctgagcttaggtcccaagcctcatcaatcacttgcaaagttgagcaggaaatatggaccagtcatgtctttgaaactggggagtctcacaactattgtcgtgtcgtccccagaagctgcaaaaatcgtgttacaaactcacgacttagccttttcttcaagatctattccgtcagctgctggtgccctaaatcatgacaagttttctatggtctggcttccagttgaaaatcaatggcgtaagttaagaaagatttgcaaagaacagatgttcagtgtttctcgtctggatgctagtcaaggattaagaagagaaaaacttcaaaagttatgtgattatgttaacgagtgttgcgaaacaggtcgagccgtggatattggtgaagctgcgtttacgacctccttgaatttgatttcagcaaccttgttctcaaaagagtttgctcagttcaacactggctcctctcaagaaatgaaagaagtagtaagaggcgtaatgaaatgtagcggtaccccaaactttgccgattatttcccagtgttaaaatcagtggatcctcaaggtatccttaaagaagctaagatatacttcgaaagattgttggctattttcgacgaaataatcgatgaaaagctcaagaatcggtcaaaaagcaccgagaaaaacgatttggtagatgcgctgatagaaattaaccaaagagacgaagccgaattaagtaggaacgatataaaacacttattgttagatctatttgtcgcaggtagtgatacaacttccggtacagttgaatgggctatgacggaactattgaggaatcctgaaaaaatgtcgagagtcaggggagagatcattgatataattggtgccaacggacaagttcaagagtctgatatttcgaatcttccctatcttcaggcagtagttaaggaaacatttagattacatccagccggcccatttctagttccgcataaagcggaagctaatgttgaagtcaatgggtacatagtaccaaaatctgcacagatactggtcaatgtttgggcatcgggtagagacagtaacgtttggtccaatgcagactcttttatgccagaacgattcttagatcatcaaatcgatttccgtgggaaggactttgagttgataccttttggtgctggaagaaggatttgtccaggattgcctttggcttaccgcatggttcacctaatggtggcgacactagtttacaatttcaaatggaagttagaggaaggcactaaacctgaggagatcgacatgaatgagaaatttggtctatccttgcagaagactatcccgctcaaagcaattcctacgaagtaa

SEQ ID NO: 26 - protein, 457 aa, Buddleja sp.
MFPIIGNILSLGPKPHQSLAKLSRKYGPVMSLKLGSLTTIVVSSPEAAKIVLQTHDLAFSSRSIPSAAGALNHDKFSMVWLPVENQWRKLRKICKEQMFSVSRLDASQGLRREKLQKLCDYVNECCETGRAVDIGEAAFTTSLNLISATLFSKEFAQFNTGSSQEMKEVVRGVMKCSGTPNFADYFPVLKSVDPQGILKEAKIYFERLLAIFDEIIDEKLKNRSKSTEKNDLVDALIEINQRDEAELSRNDIKHLLLDLFVAGSDTTSGTVEWAMTELLRNPEKMSRVRGEIIDIIGANGQVQESDISNLPYLQAVVKETFRLHPAGPFLVPHKAEANVEVNGYIVPKSAQILVNVWASGRDSNVWSNADSFMPERFLDHQIDFRGKDFELIPFGAGRRICPGLPLAYRMVHLMVATLVYNFKWKLEEGTKPEEIDMNEKFGLSLQKTIPLKAIPTK

SEQ ID NO: 27 - DNA, 1488 bp, synthetic construct
  source 1..1488: mol_type = other DNA
atggattttccgacttttctattggttgtattgtcaatcatttggattagtaccatgatcttgaccttcaatagtcgcgcccgcaagtcgtctaaattgccgccaggacctaatagattcccaattatcggaaacattttggagattggtccaaaaccgcataaatccttagctaaacttgcggcaaaatatggtccagtcatgtcattaaagttgggtacgttaacaacagtgatcatcagtagccctaaagcagctaaggctgtgttacaaactcatgatctaattttttcttcaagaaccgttccttgtgcagcagagattttatctcatcatgagtcgtcaatcgcctggttgccagccgaaaatagatggcgtaaattaagaaaactttgtcaagagcaaatgttttccgttgcaaggttagatgcctctcaaaatttacgacgagagaagctaagaaagcttaggggttacgttggtgcttgtagtgaaacagggagagcggtcgacataggggatgcagcatttactacggcgctaaacttaatgagtgctactttattttccttagaatttgcaccagaatttgactctgactcatcacaagaaatgaaagacgtggtctggggtgttatgaaatgcactgggagcccaaatttcgccgactacttccccattctaaaaagcgtagaccctcagggaatattaaaggaagctaagacgtatttccagaagctcttcgtaatatttgacggcattatagacgacaaattgaaaagcggtggagagtctggcaagcaggatttactcgaagcactgatagaaattaaccgtagggatgagaacgaattgacaaaaaatgatatcaagcacctgcttttagatttgttcgtagcaggtactgacaccacttctgtgacagttgaatgggctatggctgaattgcttaaacacccccataagatgtcccgtgtccggcaagaaatacgtgatactgtcggtgaaaacgtggaagttgaagaatccgatatttctagacttccatatctgcaagctgtagtgaaagagacatttaggctacacccagctgctccttttcttgtcccccacaaagttaatgctgatgttgaaataaaaggttttacagttccaaaaaacgcacaagttctggttaatgtatgggcttcgggcagggatcctgatacttggcccgaagccgaatcattttcccctgaaagattcatggatggaagacaaatagatattagaggaaaagattttgaattgattcctttcggtagtggcaggagaatatgtcctggtctgccgctagcctacagaatggtccatttgatgttggcgaatttagttgggtctttcgagtggagactcgatggcgattgcaaggaaatcgatatggatgacaaatttggtctgaccttgcagaaggctattccactaaaggccgtaccaatgaagttgtaa

SEQ ID NO: 28 - protein, 495 aa, Erythranthe guttata
MDFPTFLLVVLSIIWISTMILTFNSRARKSSKLPPGPNRFPIIGNILEIGPKPHKSLAKLAAKYGPVMSLKLGTLTTVIISSPKAAKAVLQTHDLIFSSRTVPCAAEILSHHESSIAWLPAENRWRKLRKLCQEQMFSVARLDASQNLRREKLRKLRGYVGACSETGRAVDIGDAAFTTALNLMSATLFSLEFAPEFDSDSSQEMKDVVWGVMKCTGSPNFADYFPILKSVDPQGILKEAKTYFQKLFVIFDGIIDDKLKSGGESGKQDLLEALIEINRRDENELTKNDIKHLLLDLFVAGTDTTSVTVEWAMAELLKHPHKMSRVRQEIRDTVGENVEVEESDISRLPYLQAVVKETFRLHPAAPFLVPHKVNADVEIKGFTVPKNAQVLVNVWASGRDPDTWPEAESFSPERFMDGRQIDIRGKDFELIPFGSGRRICPGLPLAYRMVHLMLANLVGSFEWRLDGDCKEIDMDDKFGLTLQKAIPLKAVPMKL

SEQ ID NO: 29 - DNA, 1491 bp, synthetic construct
  source 1..1491: mol_type = other DNA
atggcatggttctggcaaacaatcttgatcatactgttgaattggatcattcagaaattccttacattgaagaagaagcaaccaccgggacccattggtttacccatcataggccattttcacctcctggggaagaaccctcatcaagatctacatagactggctcaaaaacatggacctatcatgtatttgagatttgggtttgttcctactgttgtttttagctcaccagcaggtgctgaacttgttcttaagacgcatgatttagtctttgccggaagggcttcaaatcaagctgcgaagcatatctcatacgatggacgtaatattgttttcgctcaatatggatcatactggcgcgatatgcgaaagttagctactttaaatttactgagcactagtaaagtgagtcaattccaaccagttagaaaagccgaattgaggttggcggttgattcgttaagacgggctgcagcaggtcgtgaaactgttgacgtttctgaaatgattatgggtacaataggtgatatggtatgtttgatggtatttgggagaaaatttgctggtggtgaattaggtggttctggtggtggttttaaatatgttatagatgaaacatttcaagtggcagcaaaaccaaatcttggcgattttttcccttatatgggcactattgacctacaaggccttaccagaaggatgaaggagttgtccacaaccttcgatggatttttagagaagataatcgacgaccacgtacttcaaaacaagcaacagaaaaaagaaaaccaggacttcgtggacactatgatggccattatggatagcggtgaagcaggtttcgaattcgataggagacatgtcaaagcagttttacttgacatgctattgggcggtacggatacatcatcggccaccattgattgggccctggctgaactgattcgtcatccacagattatgaaaaagctacaacaggaacttgagcaggtggtcgggttggaccaatacgtggaggaaagtcacttagaaaagttagactacttgaatttcgtcgtgaaggagacgtttcgcttacatcccgtagttcctttgtttatacatgaagccctagaagactgtactgtggaggaatttcatattgctaaagggacacgaattatggttaacgtatggtctatgtccaaagatcctaatgtctggcaggatcctgagaaattctttccagaaaggtttataggttctaacttagatttgcacggtcacgattttgaactattgccattcggtagtggcagaagatcctgcccgggcttacaactaggattgaccactgttagattggtactagcgcaattggtacactgcttcgattgggagctcccgaatggtatgtctccatccgaattagatatgtctgaacattttggactcgtcaccggaaaagataaacacattatggctattccagtctgtagattacataaataa

SEQ ID NO: 30 - protein, 496 aa, Erythranthe guttata
MAWFWQTILIILLNWIIQKFLTLKKKQPPGPIGLPIIGHFHLLGKNPHQDLHRLAQKHGPIMYLRFGFVPTVVFSSPAGAELVLKTHDLVFAGRASNQAAKHISYDGRNIVFAQYGSYWRDMRKLATLNLLSTSKVSQFQPVRKAELRLAVDSLRRAAAGRETVDVSEMIMGTIGDMVCLMVFGRKFAGGELGGSGGGFKYVIDETFQVAAKPNLGDFFPYMGTIDLQGLTRRMKELSTTFDGFLEKIIDDHVLQNKQQKKENQDFVDTMMAIMDSGEAGFEFDRRHVKAVLLDMLLGGTDTSSATIDWALAELIRHPQIMKKLQQELEQVVGLDQYVEESHLEKLDYLNFVVKETFRLHPVVPLFIHEALEDCTVEEFHIAKGTRIMVNVWSMSKDPNVWQDPEKFFPERFIGSNLDLHGHDFELLPFGSGRRSCPGLQLGLTTVRLVLAQLVHCFDWELPNGMSPSELDMSEHFGLVTGKDKHIMAIPVCRLHK

SEQ ID NO: 31 - DNA, 1488 bp, synthetic construct
  source 1..1488: mol_type = other DNA
atggcttggatctggataagtctttgcaccataattgttatctatcttctccaagagcttgtcaacctcaacaagaagaacaaattgcctccaggaccaatagcactacctatcttaggtcatattcatttactgggtaaaaatccacaccagaatttacatcatttggcgcaaaaacacggcccaataatgttcatgcgttttggattcaaacccgtcatcgttatatcatcaccgggtgccgcagaactggttttacgaacccatgatctagtatttgcctgtagaccacactctcaagcgtctaagcatcttggctacaatagaaagaatataattttcgccccatacggtccctattggaggaatatgaggaagttgtgcactttacaattgttatcacaacttaaaattaatcaattccagcccatgagaagaactgagctaggattcttagtttccagcctaaagcaagcagctgagataggcgaaacagtagatatttcggctcaaatttcaaccttgaatagggatatgacatgtttgatggtgtttggtagaaaatttggcgatggtgatttggacgaaaagggtttcaaggctctggctgacgaaatagttcaagtagcagctgtgcctaatttaggtgactatttcccattgatgggattcctggatctacagggtttaacacgccggatgaaagaactttcaaaaacgttcgaccgttttttggacaagattattgatgaccacattcagtgcaacaaccaagagaaggaaaatcaggactttgtcgatactctgatgaatattatgatgtctggggaagctggaggtgtgttggaatttgatagagttcatgtcaaagcggttttattagatatgcttattggtgggatggatacatctgctgctgcagttgagtggactttgtcggaactcatcaggcatccaaaagccatgaaaaaattgcaaaaagagttgaaagacgttgtagggatggatcagaccgtggacgaatctcacttggataagctagaatacctagactttgttctgaaggaaacgttaagattgcatcccgtcgggccgttactaattcacgagagtattgaagactgtaaagtagatgaatttcatatcccaaaaggctcccagctaattgttaacgcctggagcatcggtagggatcctaatgtgtggaaaaacccggaaaaatttattcctgaaagatttatcggttctgatattgatttgaagggacaacattttgaactgctcccttttggcagtggaagacgtggttgtccaggtatgcaattaggtttgatagtagtgagattgatggtcgcacaattagtccactgtttcgattgggaattgcataacggaatgttagcatccgagcttgatatgtccgaacattttggcatagtaactggtagagatacacacttaatggttatccctacttatcgattataa

SEQ ID NO: 32 - protein, 495 aa, Verbascum sp.
MAWIWISLCTIIVIYLLQELVNLNKKNKLPPGPIALPILGHIHLLGKNPHQNLHHLAQKHGPIMFMRFGFKPVIVISSPGAAELVLRTHDLVFACRPHSQASKHLGYNRKNIIFAPYGPYWRNMRKLCTLQLLSQLKINQFQPMRRTELGFLVSSLKQAAEIGETVDISAQISTLNRDMTCLMVFGRKFGDGDLDEKGFKALADEIVQVAAVPNLGDYFPLMGFLDLQGLTRRMKELSKTFDRFLDKIIDDHIQCNNQEKENQDFVDTLMNIMMSGEAGGVLEFDRVHVKAVLLDMLIGGMDTSAAAVEWTLSELIRHPKAMKKLQKELKDVVGMDQTVDESHLDKLEYLDFVLKETLRLHPVGPLLIHESIEDCKVDEFHIPKGSQLIVNAWSIGRDPNVWKNPEKFIPERFIGSDIDLKGQHFELLPFGSGRRGCPGMQLGLIVVRLMVAQLVHCFDWELHNGMLASELDMSEHFGIVTGRDTHLMVIPTYRL

SEQ ID NO: 33 - DNA, 1266 bp, synthetic construct
  source 1..1266: mol_type = other DNA
atggcctggatatgggtatccattagcttgataatttttctctggattttgcataatcagtttacgtcgaacagaaaaaagaaatttcccccagggcctatcgggttgcctgttttgggccatcttcatcttttaggtaaaaatcctcaccacgatttacatcgattagcgcagaagcacggtccgataatgtcattacggtttggactagttccaactgttgttgtctcttcaccagctggtgctgaattaatcttaaaaacacacgacttggtatttgcaggaagaccccgttatcaagccgccaagcatctctcttatggtggcagaaacattcttttcgctccatacggcaactattggaggaacatgagaaaattatgcacactagaactgctatccagctccagaatcaaccaattcagaccgataagaacagtggagttgggttttttaattaaggctctacaaagtgcagcagagaggggagagactgtggatgttagtgcacgagtaagtgctttaaatggcgatgttatttcattaatggtgttcggtcgcaagtttgatgataatgaattggacgaaaggggtttcaaggccgtcatagaggaaaccctggctatggcagcaaggcctaatctaggtgatttctttccattgattgcagctgtcgatcttcaaggtttagatagaagaatgaaggaattaagcggaatgtttgatggtttcctcgaacgcattatagaagaccatcgtggtaaaggcaaaagaaagaaggaagaacaggaagacttcgtcgatacaatggtcggtattttagaatctggagaagcggaattcgagtttgatattagacacgtaaaagctgttctaatggatatgctaattggtggaatggacacttctactactgccgttgaatgggtattgtcagaattgatcagacatcccaaagctatgaagaaactgcaacaggaactggagcaagtcgttggcatcgaccaattggtggatgagtcacatttggacaaattggaataccttaatatgacgataaaagagaccctgaggcttcatcctgtgggaccattgcttatccacgaatctatggaggactgtaccgtcagtgggttccatatacgtaaaggttccagaattattgtaaatgtttcttcgatcggtaaagatccaaatgtttggcaagatcctgaaaagtttatgccagaacgttttgtggggtctcatgttgatttaaggggttaa

SEQ ID NO: 34 - protein, 421 aa, Sanchezia sp.
MAWIWVSISLIIFLWILHNQFTSNRKKKFPPGPIGLPVLGHLHLLGKNPHHDLHRLAQKHGPIMSLRFGLVPTVVVSSPAGAELILKTHDLVFAGRPRYQAAKHLSYGGRNILFAPYGNYWRNMRKLCTLELLSSSRINQFRPIRTVELGFLIKALQSAAERGETVDVSARVSALNGDVISLMVFGRKFDDNELDERGFKAVIEETLAMAARPNLGDFFPLIAAVDLQGLDRRMKELSGMFDGFLERIIEDHRGKGKRKKEEQEDFVDTMVGILESGEAEFEFDIRHVKAVLMDMLIGGMDTSTTAVEWVLSELIRHPKAMKKLQQELEQVVGIDQLVDESHLDKLEYLNMTIKETLRLHPVGPLLIHESMEDCTVSGFHIRKGSRIIVNVSSIGKDPNVWQDPEKFMPERFVGSHVDLRG

SEQ ID NO: 35 - DNA, 1485 bp, synthetic construct
  source 1..1485: mol_type = other DNA
atgaactggctatggaccatattgagtgctattgtattcctgaatctatttcagaggttattatcattaaagaaaaagaaacgcctgcctcccggtccacggggtttgccagttctaggccattttcacttgttggggaagaacccacatcaagatttgtgtagattagctagaaaacatggcccaataatgtaccttaggtttggtttggtacctacaatagtcgtctcaagtccagcagcagcagaacttttcttaaagacgtacgacttagtatttgcctcgcgacctcctcatcaggcttcaaaatatatcagctacgatcaaagaaacctaacctttggaccttacggtccatattggcgagatatgagaaagctgtgcactctcgagctattgagtagtctaagaatctcccaattccaagcgatgaggaaagccgaattagagttgttggtgaattccctcaaacaagcagctttgaaagaagagacggtagatttgagttctaaagtttcatcgctctcagccgacatgacatgcttgatggtcttcggaagaaagtatgtggacagagatttagatgagaaaggcttcaaagccgttatcatggatgctcttgaagtggcagctgcatttaaccttggagactatttcccgtatttgggagttttggatttacaagggctgacaagaaggatgaaagcgttacacaagatcttcgacggttttttagagaagatcattgacgaccacgtaagggataaacaggacaagaaacaatctcgtgatttcgtagatgttatgatgggtataatggaatcaggtgaagctggttttgaatttgatcgtagacacattaaggcggttttattggatatgctggtggccggaatggatacttcggctactacagtagaatggactttatccgaattgcttaagcatccgaccattatgagaaagttgcagaaagaactcgaagatgtagtgggtatggaccacatggtcgaagaatctcatcttgaccaattagaatacttagatctggtgataaaggaagccctgagacttcatcccgttgcacccttacttttgcctcattggtctatggaggactgtgttgttgatggctttcatataccgaaaaacagcagaattctagttaatgtctgggctattggtagagaccccaatgtctggccagatccagaagcattctctccagaacgttttgttggttgtaatattgatctgaggggcagagattttcaattaattccatttggaagcggtaggagaggttgtcctggcttacaactagggttaacagttgttcgtctaatggtcgcacagttggtccactgctttgattggaaattaccttccgggatgaaacctaatgagctagatatgtccgaacatttcggtttggttacttctcgtgctaatcatgtgatggctattccatcttatcgcctttaa

SEQ ID NO: 36 - protein, 494 aa, Anticharis glandulosa
MNWLWTILSAIVFLNLFQRLLSLKKKKRLPPGPRGLPVLGHFHLLGKNPHQDLCRLARKHGPIMYLRFGLVPTIVVSSPAAAELFLKTYDLVFASRPPHQASKYISYDQRNLTFGPYGPYWRDMRKLCTLELLSSLRISQFQAMRKAELELLVNSLKQAALKEETVDLSSKVSSLSADMTCLMVFGRKYVDRDLDEKGFKAVIMDALEVAAAFNLGDYFPYLGVLDLQGLTRRMKALHKIFDGFLEKIIDDHVRDKQDKKQSRDFVDVMMGIMESGEAGFEFDRRHIKAVLLDMLVAGMDTSATTVEWTLSELLKHPTIMRKLQKELEDVVGMDHMVEESHLDQLEYLDLVIKEALRLHPVAPLLLPHWSMEDCVVDGFHIPKNSRILVNVWAIGRDPNVWPDPEAFSPERFVGCNIDLRGRDFQLIPFGSGRRGCPGLQLGLTVVRLMVAQLVHCFDWKLPSGMKPNELDMSEHFGLVTSRANHVMAIPSYRL

SEQ ID NO: 37 - DNA, 33 bp, synthetic construct
  source 1..33: mol_type = other DNA
aaaaaggtttatatacaattacaatagaccctt

SEQ ID NO: 38 - DNA, 60 bp, synthetic construct
  source 1..60: mol_type = other DNA
taatgcctttatgcagtttttttttcccattcgatatttctatgtggggtacacacgact

SEQ ID NO: 39 - DNA, 31 bp, synthetic construct
  source 1..31: mol_type = other DNA
gataacttctgaatactttctaaactgatca

SEQ ID NO: 40 - DNA, 60 bp, synthetic construct
  source 1..60: mol_type = other DNA
gcagtttttttttcccattcgatatttctatgtcaatttgtacaattccccattagagtc

SEQ ID NO: 41 - DNA, 30 bp, synthetic construct
  source 1..30: mol_type = other DNA
tatgtgcgactatactagtattttaatatt

SEQ ID NO: 42 - DNA, 53 bp, synthetic construct
  source 1..53: mol_type = other DNA
gcagtttttttttcccattcgatatttctatgttctaggcctgcataatcggc

SEQ ID NO: 43 - DNA, 60 bp, synthetic construct
  source 1..60: mol_type = other DNA
gccaccgtgctcagcaaacgcttgcaaatcactccccatctcgagctttacttgtggaag

SEQ ID NO: 44 - DNA, 32 bp, synthetic construct
  source 1..32: mol_type = other DNA
ccagggaaaaacaaaattatatagaaaagaag

SEQ ID NO: 45 - DNA, 56 bp, synthetic construct
  source 1..56: mol_type = other DNA
ctcagcaaacgcttgcaaatcactccccatctcgaaggaatagttcaaagttggcg

SEQ ID NO: 46 - DNA, 60 bp, synthetic construct
  source 1..60: mol_type = other DNA
agaccgtgaaatctctttgcggtagttaaattacaatatctataaatgagaacttgacaa

SEQ ID NO: 47 - DNA, 54 bp, synthetic construct
  source 1..54: mol_type = other DNA
ctcagcaaacgcttgcaaatcactccccatctccaagttgcctgcgagtttctc

SEQ ID NO: 48 - DNA, 28 bp, synthetic construct
  source 1..28: mol_type = other DNA
ctctcttaaacacatagtaacagcaaaa

SEQ ID NO: 49 - DNA, 23 bp, synthetic construct
  source 1..23: mol_type = other DNA
tcgatgccatcggcaataatacg

SEQ ID NO: 50 - DNA, 22 bp, synthetic construct
  source 1..22: mol_type = other DNA
ggctgttccttcatagcccttt

SEQ ID NO: 51 - DNA, 28 bp, synthetic construct
  source 1..28: mol_type = other DNA
ttggatatcagtaatgagacgaaaaagc

SEQ ID NO: 52 - DNA, 26 bp, synthetic construct
  source 1..26: mol_type = other DNA
aaccctgtgccctttctatttttatg

SEQ ID NO: 53 - DNA, 30 bp, synthetic construct
  source 1..30: mol_type = other DNA
aaatgtagttaagttttcgttaaacttttc

SEQ ID NO: 54 - DNA, 23 bp, synthetic construct
  source 1..23: mol_type = other DNA
attctcgccaaggcattaccatc

SEQ ID NO: 55 - DNA, 23 bp, synthetic construct
  source 1..23: mol_type = other DNA
agtgatcttgctcatcacggaac

SEQ ID NO: 56 - DNA, 20 bp, synthetic construct
  source 1..20: mol_type = other DNA
accggtcgcgttcctgaaac

SEQ ID NO: 57 - DNA, 50 bp, synthetic construct
  source 1..50: mol_type = other DNA
gggaaagagaaaagaaaaaaattgatctatcgatttcaattcaattcaat

SEQ ID NO: 58 - DNA, 60 bp, synthetic construct
  source 1..60: mol_type = other DNA
gaaggtcgctatctcagagacgtatggtaaacgggaagtctttacagttttagttaggag

SEQ ID NO: 59 - DNA, 60 bp, synthetic construct
  source 1..60: mol_type = other DNA
ctcctaactaaaactgtaaagacttcccgtttaccatacgtctctgagatagcgaccttc

SEQ ID NO: 60 - DNA, 60 bp, synthetic construct
  source 1..60: mol_type = other DNA
cacttgctctagtcaagacttacaattaaaatgacctccgcactctacgcatccgatttg

SEQ ID NO: 61 - DNA, 60 bp, synthetic construct
  source 1..60: mol_type = other DNA
caaatcggatgcgtagagtgcggaggtcattttaattgtaagtcttgactagagcaagtg

SEQ ID NO: 62 - DNA, 60 bp, synthetic construct
  source 1..60: mol_type = other DNA
taccacgagacagggggttaagaaatccattgtttaagtgggtgatgttggaggtatttg

SEQ ID NO: 63 - DNA, 60 bp, synthetic construct
  source 1..60: mol_type = other DNA
caaatacctccaacatcacccacttaaacaatggatttcttaaccccctgtctcgtggta

SEQ ID NO: 64 - DNA, 60 bp, synthetic construct
  source 1..60: mol_type = other DNA
aactactaacccgttagtggccaaatctacttataattttgttggaatcgctcgtagtgg

SEQ ID NO: 65 - DNA, 60 bp, synthetic construct
  source 1..60: mol_type = other DNA
ccactacgagcgattccaacaaaattataagtagatttggccactaacgggttagtagtt

SEQ ID NO: 66 - DNA, 60 bp, synthetic construct
  source 1..60: mol_type = other DNA
caatgagagagccgcccaaaaccactccattgtttaagtgggtgatgttggaggtatttg

SEQ ID NO: 67 - DNA, 60 bp, synthetic construct
  source 1..60: mol_type = other DNA
caaatacctccaacatcacccacttaaacaatggagtggttttgggcggctctctcattg

SEQ ID NO: 68 - DNA, 60 bp, synthetic construct
  source 1..60: mol_type = other DNA
gctattccaacctacagattaaataaataagtagatttggccactaacgggttagtagtt

SEQ ID NO: 69 - DNA, 60 bp, synthetic construct
  source 1..60: mol_type = other DNA
aactactaacccgttagtggccaaatctacttatttatttaatctgtaggttggaatagc

SEQ ID NO: 70 - DNA, 29 bp, synthetic construct
  source 1..29: mol_type = other DNA
tactttaacgtcaaggagaaaaaactata

SEQ ID NO: 71 - DNA, 60 bp, synthetic construct
  source 1..60: mol_type = other DNA
taaaatcactgagctagttaaaaaatccattgtttaagtgggtgatgttggaggtatttg

SEQ ID NO: 72 - DNA, 60 bp, synthetic construct
  source 1..60: mol_type = other DNA
caaatacctccaacatcacccacttaaacaatggattttttaactagctcagtgatttta

SEQ ID NO: 73 - DNA, 60 bp, synthetic construct
  source 1..60: mol_type = other DNA
aactactaacccgttagtggccaaatctacttatactttaattggaatagctttcaaagg

SEQ ID NO: 74 - DNA, 60 bp, synthetic construct
  source 1..60: mol_type = other DNA
cctttgaaagctattccaattaaagtataagtagatttggccactaacgggttagtagtt

SEQ ID NO: 75 - DNA, 60 bp, synthetic construct
  source 1..60: mol_type = other DNA
caaatacctccaacatcacccacttaaacaatggattttccgacttttctattggttgta

SEQ ID NO: 76 - DNA, 60 bp, synthetic construct
  source 1..60: mol_type = other DNA
tacaaccaatagaaaagtcggaaaatccattgtttaagtgggtgatgttggaggtatttg

SEQ ID NO: 77 - DNA, 60 bp, synthetic construct
  source 1..60: mol_type = other DNA
ccactaaaggccgtaccaatgaagttgtaagtagatttggccactaacgggttagtagtt

SEQ ID NO: 78 - DNA, 60 bp, synthetic construct
  source 1..60: mol_type = other DNA
aactactaacccgttagtggccaaatctacttacaacttcattggtacggcctttagtgg

SEQ ID NO: 79 - DNA, 8007 bp, synthetic construct
  source 1..8007: mol_type = other DNA
aacttgccacagttcagcagaaagaatcagagttgaaagaattcataaacgatgtagaagaagaagatggtgtacctcaatatgccgcttcaagtgatgaagaaggcgacgaaaatgtgtcaaaaaagagaaaaatggagagcccaccaactgaaacagttgattctggcgaacagccaactaacccagtccaccctcagctttcctccatcttagagagtctatctagaacaactggactaacaccagaacctgcttctacttcggacgagtcagcaacagcaacgcagaagcaagatgaaacacctgtttcttcatctataaatcctgctctagccagtttgctgtccaaacttaacggttaattcttaatgtcttccccaatcacttgagtacgaactatgtattatataatcaggcatcttttctttttttcttttttttttgtctctcgcttggatcccaacaccatatttcagatctcctgatgactgactcactgataataaaaatacggcttcagaatttctcaagactacactcactgtccgacttcaagtgacccttgtgactgacactttgggagtccctattctacttagtctcatatcgcatgaaacttttgataaattattttctgataggaatttttcatcagatattatcatcgcggcttacgtaataacaaaaaaaattgatggagtctatactaggctaacataaactaagttattaattaaacaaaacaaaacgtactagcattactgtcatatataagggctcctaactaaaactgtaaagacttcccgtttaccatacgtctctgagatagcgaccttccgtttgtaacttcttaacgatggcttcagcttcggatgaagatacaccttcttgttcttgcactatcgtatgtaatgtacgatgaacatctctagccatacctttggcatcaccgcaaacatatagatatccttcctccttgatcaagtcccaaacttgcgcagccttctccatcattttatgctgaacatattccttttgtgctccctctctcgaaaaggccataattaattcggaaatcacgccctgatcgacgaaattgttaagctcgtcttcgtaaatgaaatccatttgcctgttccgacagccaaagaacagtaaactggagcctaattcttcaccgtcttctttcagcgccattctttcctgcaaaaaaccacggaaaggtgctagaccggttccgggacccaccataactataggtgtagacgggttcgagggcaacttaaaattagaagctcttataaagataggggcaccagaacattcgtgagacttttcagctgggactgcatttttcatccaagtggaacaaacacctttatggatcctacctgttggggttggaccataaaccaacgctgaagtgacatgaactctgctgggtgccaaccttggagaagaagaaatactgtaatatcttggttgcaatctcggggcaatggcagcgaaaaagactccaagtggaggtttggcagagggaaacgctgccatcacttctaataatgatctttgtgatgcaacaatccactgtgagtattcgtctttaccatcaggactggtgagatgttttaatttttctgcttcactaggttcagtggcatatgcagctaaagctactagtgctgattttcgtggcggattcaataaatctgcatacctagccagaccggttcctaaagtacaaggcccaggaaatggtggtggtacagcactttccaaaggtgagccatcttctttatcagcatgaatcgagaaaactaaatctaaggagtgccccagtagtttaccagcttcttcaacaatctcgacatgattctcagcatagacaccaacatgatctcctgtctcataagttatccccgttctgctgatatcaaactccaaatgtatacagctcctatcagattcatgggtatgaagctctttttgtactgctacatcaactctgcaagggtggtgaatatcaatagtagtgttaccattggcgacatttgattccattgacttttgagttgtgaaacgtggatcgtgtgttacgactctatactcgggaataacggcagtatatggtgtagccacactcttatcatcttcgtcctttaaaagtttatccaattcactccacaacgattctttccacgcgttgaaatcatcctcaatgctttgatcgtcatcgccgagacccacctcaataagcctttttgctccttttttgcacagctcctcatctagaacgatcccgatcttattaaagtgttcgtattgtcggttgccaagggcaaacaccccgtatgcaagttgctgtaattttatgtcacgttcgttctcttcagtgaaccacttgtagaatctagcagcattgtctgtcggttctccatcgccgtatgtagctacacaaaaaaaagccaacgtctccttctttagtttttcctcgtattggtcgtcgtccgcagcgtaatcgtccaggtctataaccttgactgctgccttttcatatctggccttaatttcttccgacaatgctttagcaaagccttcagccgtacctgtctgggtgccaaaaaatatagagactcttgttttacctgaacctaggtctagatcatcatcttcatctttagccatcagactctttgggatcattagtggttttaattcaccagagcgatcagcagttgtttttttccacaaaagtacaacgaatccggctactaaggccaatgaagtagtcgcaatcactaaaaccacgtcatcgctcaaggagtcggttcccattatagatttcaactgcttaaacaaatcggatgcgtagagtgcggaggtcattttaattgtaagtcttgactagagcaagtgttatggtgaatttcaagtaattgaagtgctgggtctacagcttatatatgattttggcctgatgagcaacagaggctatcaccccgctgggataatgaaacttaccgattgcggcaggctaagcgaaaagtccaggccaatcgagggaagttaattgctcagctttcctgactaatcccaactttgaggggtgcatagtatggattcggtctcgtgcgagaagcttaaactttgttcaattccattagtagagggtatcatggaacttgcatgtgatgattacttcatatgatatcaggtactagcaatctcgattcgaatttattaggtaatgccgaccggaacaatagaaagacttcaagattgctaaaagaccaataagaacgtgtacagatgtcaggccatgagtttatttaagtgatctatgctacaattaaggttacaggagaaccgcattcggagttcgattatgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggatttcttaaccccctgtctcgtggtagcatccattgcctggatttgtatgttgatactaagagcaagaaaaccgtctaaacttcctcctggcccatatggtccgcccatcataggtaatatactacatttaggtcctaaacctcatagatctttggccgacttggcccgaaaatatgggccagtcatgaagcttaggttgggttcggtaaccacagttgttatatcttccccagaagcagctaaagctgttttacaaaaacacgattcatcattttggaatagacctgctccttcttctgttagggcggttggtcacgatgaattctcagtcgcttggctgcctgtagacaaacaatggagaaaacttcggaaaataatgaaagaattaatgttctcatcgccaagattagatgccggccaaggtatgagacgtgcaaaacttcaacaattaagtgattatgtctggggtaggtgccaggctggtagagcggtcgaggttggagaggctgcattcacgacttctctgaatttaatgtcagctacattattttctactgattttgctcgtttcgattccgatagcagtcaggaaatgaaggaagtggtatggggcgttatgaagtgtgtgggttctccaaacttggtagattactttccagtcttgaaatccctcgatccgcaagggatcctaaaagacgcaaagttctgctttgggaagttgttcgcgatctttgacgaaattctggatgaacgcttgaagatctctcgtggtgaaaagcaggatttggtagaggcactaatcgatttaaatcaacgtgacgttcctcaactaagccgcgacgatattaaccacctattgttagacttatttgttgctggttcagatactactagtggtacagtcgaatgggcaatgacagaattgattaggcatcccgaaaagatgactaagttgagaaatgagattacttcatttgttgaagagaacggcccaattgaagaaagtgatatcagcagattgccatacttacaagccgtcgttaaagagacgtttagacttcatcctgtggccccattcctacttccacataaggctagctcggacattgaaattaacggttacacggttcctaaaaatgcacagatcctggttaacatttgggcctccggaagggatccgattaactgggtggatgctgataagtttgtgccagaaaggtttttaagtgagaataaaggactgaactttataggacaggattttgaactcataccctttggcgctggtagaagaatttgtccagggttgcccttggctaatagaatggtccatcaaatgctggtgaccttcgttggaaatttcgagtggaagcttgaaggaataaaggtagaggaaatggacatggacgaaaattttggcttaaccttgcaaaaagcaatcccactacgagcgattccaacaaaattataagtagatttggccactaacgggttagtagttgtgtaagtctattaaatttgatttttgtttatggatgatcatcgtagtggctatctgtttacctgtaggacatcctagggtgggatggtgatgtacaccccctcaatcttcagatgcaacactatgtggtaggtcattgacataaggtttaggaaagacctgttttttgaccaataaatggaacaggaaggaaaggaggaaccagtttacgaaccccgtcgatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 80 - DNA, 8028 bp, synthetic construct
  source 1..8028: mol_type = other DNA
aacttgccacagttcagcagaaagaatcagagttgaaagaattcataaacgatgtagaagaagaagatggtgtacctcaatatgccgcttcaagtgatgaagaaggcgacgaaaatgtgtcaaaaaagagaaaaatggagagcccaccaactgaaacagttgattctggcgaacagccaactaacccagtccaccctcagctttcctccatcttagagagtctatctagaacaactggactaacaccagaacctgcttctacttcggacgagtcagcaacagcaacgcagaagcaagatgaaacacctgtttcttcatctataaatcctgctctagccagtttgctgtccaaacttaacggttaattcttaatgtcttccccaatcacttgagtacgaactatgtattatataatcaggcatcttttctttttttcttttttttttgtctctcgcttggatcccaacaccatatttcagatctcctgatgactgactcactgataataaaaatacggcttcagaatttctcaagactacactcactgtccgacttcaagtgacccttgtgactgacactttgggagtccctattctacttagtctcatatcgcatgaaacttttgataaattattttctgataggaatttttcatcagatattatcatcgcggcttacgtaataacaaaaaaaattgatggagtctatactaggctaacataaactaagttattaattaaacaaaacaaaacgtactagcattactgtcatatataagggctcctaactaaaactgtaaagacttcccgtttaccatacgtctctgagatagcgaccttccgtttgtaacttcttaacgatggcttcagcttcggatgaagatacaccttcttgttcttgcactatcgtatgtaatgtacgatgaacatctctagccatacctttggcatcaccgcaaacatatagatatccttcctccttgatcaagtcccaaacttgcgcagccttctccatcattttatgctgaacatattccttttgtgctccctctctcgaaaaggccataattaattcggaaatcacgccctgatcgacgaaattgttaagctcgtcttcgtaaatgaaatccatttgcctgttccgacagccaaagaacagtaaactggagcctaattcttcaccgtcttctttcagcgccattctttcctgcaaaaaaccacggaaaggtgctagaccggttccgggacccaccataactataggtgtagacgggttcgagggcaacttaaaattagaagctcttataaagataggggcaccagaacattcgtgagacttttcagctgggactgcatttttcatccaagtggaacaaacacctttatggatcctacctgttggggttggaccataaaccaacgctgaagtgacatgaactctgctgggtgccaaccttggagaagaagaaatactgtaatatcttggttgcaatctcggggcaatggcagcgaaaaagactccaagtggaggtttggcagagggaaacgctgccatcacttctaataatgatctttgtgatgcaacaatccactgtgagtattcgtctttaccatcaggactggtgagatgttttaatttttctgcttcactaggttcagtggcatatgcagctaaagctactagtgctgattttcgtggcggattcaataaatctgcatacctagccagaccggttcctaaagtacaaggcccaggaaatggtggtggtacagcactttccaaaggtgagccatcttctttatcagcatgaatcgagaaaactaaatctaaggagtgccccagtagtttaccagcttcttcaacaatctcgacatgattctcagcatagacaccaacatgatctcctgtctcataagttatccccgttctgctgatatcaaactccaaatgtatacagctcctatcagattcatgggtatgaagctctttttgtactgctacatcaactctgcaagggtggtgaatatcaatagtagtgttaccattggcgacatttgattccattgacttttgagttgtgaaacgtggatcgtgtgttacgactctatactcgggaataacggcagtatatggtgtagccacactcttatcatcttcgtcctttaaaagtttatccaattcactccacaacgattctttccacgcgttgaaatcatcctcaatgctttgatcgtcatcgccgagacccacctcaataagcctttttgctccttttttgcacagctcctcatctagaacgatcccgatcttattaaagtgttcgtattgtcggttgccaagggcaaacaccccgtatgcaagttgctgtaattttatgtcacgttcgttctcttcagtgaaccacttgtagaatctagcagcattgtctgtcggttctccatcgccgtatgtagctacacaaaaaaaagccaacgtctccttctttagtttttcctcgtattggtcgtcgtccgcagcgtaatcgtccaggtctataaccttgactgctgccttttcatatctggccttaatttcttccgacaatgctttagcaaagccttcagccgtacctgtctgggtgccaaaaaatatagagactcttgttttacctgaacctaggtctagatcatcatcttcatctttagccatcagactctttgggatcattagtggttttaattcaccagagcgatcagcagttgtttttttccacaaaagtacaacgaatccggctactaaggccaatgaagtagtcgcaatcactaaaaccacgtcatcgctcaaggagtcggttcccattatagatttcaactgcttaaacaaatcggatgcgtagagtgcggaggtcattttaattgtaagtcttgactagagcaagtgttatggtgaatttcaagtaattgaagtgctgggtctacagcttatatatgattttggcctgatgagcaacagaggctatcaccccgctgggataatgaaacttaccgattgcggcaggctaagcgaaaagtccaggccaatcgagggaagttaattgctcagctttcctgactaatcccaactttgaggggtgcatagtatggattcggtctcgtgcgagaagcttaaactttgttcaattccattagtagagggtatcatggaacttgcatgtgatgattacttcatatgatatcaggtactagcaatctcgattcgaatttattaggtaatgccgaccggaacaatagaaagacttcaagattgctaaaagaccaataagaacgtgtacagatgtcaggccatgagtttatttaagtgatctatgctacaattaaggttacaggagaaccgcattcggagttcgattatgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggagtggttttgggcggctctctcattgatagtctttttgtcgttgttacaccaattactaaaaaaggagaagaaaagggaagctaacctcccaccgagtcctattgctcttcctgtaatcggccatttacacctattgggaaaaaaccttcccttaaagctgcacgcaattgccgaacgtcatggtcctatagtttttttaagattaggccttgtacgtgccttggtggttagcacagcagctggggcggagttagtgttaaaaactcatgatttggtttttagtggaagagttcatcatcaagcatctcgctaccttggttatgaccaaaagaatatcgtatttgcaccttatggggcatattggcgaaacatgcgtagattatgtatggtcaaattgttgaatgctgccaagataaatgaatttaggcccgtaagaagagctgaattagaagaaaccgttgcttccatgagaagggcagctgaagaaaggggagtggtggacgtgtcagcagtaatttccggtgtcattggagatatgaactcgttaatggttttcggaagaaaatatgtggaccgggatctggatgaagagttaggtttcaaggcagtcatagatgaaatgttgcacgttggtgccttgcctaatctaggtgacttttttccattcatggctgcactagacttgcaaggtttggatagaaggatgaaagaattgagcaagatttttgacgggttcctagaacgtataattgacgatcacttgctgaaaaagactgagaataccaaaaagggggatttcgtggatacaatgctggctgttatggaggccggcgaagccgattttgaattcgatcgtagacatgttaaagctgttctgctcgatatgcttatcgctggtatggatacgtctgccagcacagttgaatgggctctgtctgagttgattagacaccctgaaatcactaagaaactacaaaaagaactcgagcaggttgttggtatggaccagatggtagatgagtctcatctagacaaactagattacctggattccgtccttaaagagaccttaagattatacccacccggtagattgcttgtccatgaaacgatggaagaatgtacagttaacggttttcatattccaaagggtacatggactttcgtcaatatgtggagtataggaagagatccagcgatgtggcatgaaccagaaaagtttgtacctgaacgctttgccggtgaaaatttagattttctaggccaaaatttcaaattcattccattcggtgcgggccgaaggtcatgcccgggtttacagttgggcttaacttttgttaggttggtgttggcacaacttgtacattgttttgactgggaacttccaaacggaatggttccgtcagacttggatatgaacgagaaattcggtatcgtcacttctagagataagcatttaatggctattccaacctacagattaaataaataagtagatttggccactaacgggttagtagttgtgtaagtctattaaatttgatttttgtttatggatgatcatcgtagtggctatctgtttacctgtaggacatcctagggtgggatggtgatgtacaccccctcaatcttcagatgcaacactatgtggtaggtcattgacataaggtttaggaaagacctgttttttgaccaataaatggaacaggaaggaaaggaggaaccagtttacgaaccccgtcgatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 81 - DNA, 8010 bp, synthetic construct
  source 1..8010: mol_type = other DNA
aacttgccacagttcagcagaaagaatcagagttgaaagaattcataaacgatgtagaagaagaagatggtgtacctcaatatgccgcttcaagtgatgaagaaggcgacgaaaatgtgtcaaaaaagagaaaaatggagagcccaccaactgaaacagttgattctggcgaacagccaactaacccagtccaccctcagctttcctccatcttagagagtctatctagaacaactggactaacaccagaacctgcttctacttcggacgagtcagcaacagcaacgcagaagcaagatgaaacacctgtttcttcatctataaatcctgctctagccagtttgctgtccaaacttaacggttaattcttaatgtcttccccaatcacttgagtacgaactatgtattatataatcaggcatcttttctttttttcttttttttttgtctctcgcttggatcccaacaccatatttcagatctcctgatgactgactcactgataataaaaatacggcttcagaatttctcaagactacactcactgtccgacttcaagtgacccttgtgactgacactttgggagtccctattctacttagtctcatatcgcatgaaacttttgataaattattttctgataggaatttttcatcagatattatcatcgcggcttacgtaataacaaaaaaaattgatggagtctatactaggctaacataaactaagttattaattaaacaaaacaaaacgtactagcattactgtcatatataagggctcctaactaaaactgtaaagacttcccgtttaccatacgtctctgagatagcgaccttccgtttgtaacttcttaacgatggcttcagcttcggatgaagatacaccttcttgttcttgcactatcgtatgtaatgtacgatgaacatctctagccatacctttggcatcaccgcaaacatatagatatccttcctccttgatcaagtcccaaacttgcgcagccttctccatcattttatgctgaacatattccttttgtgctccctctctcgaaaaggccataattaattcggaaatcacgccctgatcgacgaaattgttaagctcgtcttcgtaaatgaaatccatttgcctgttccgacagccaaagaacagtaaactggagcctaattcttcaccgtcttctttcagcgccattctttcctgcaaaaaaccacggaaaggtgctagaccggttccgggacccaccataactataggtgtagacgggttcgagggcaacttaaaattagaagctcttataaagataggggcaccagaacattcgtgagacttttcagctgggactgcatttttcatccaagtggaacaaacacctttatggatcctacctgttggggttggaccataaaccaacgctgaagtgacatgaactctgctgggtgccaaccttggagaagaagaaatactgtaatatcttggttgcaatctcggggcaatggcagcgaaaaagactccaagtggaggtttggcagagggaaacgctgccatcacttctaataatgatctttgtgatgcaacaatccactgtgagtattcgtctttaccatcaggactggtgagatgttttaatttttctgcttcactaggttcagtggcatatgcagctaaagctactagtgctgattttcgtggcggattcaataaatctgcatacctagccagaccggttcctaaagtacaaggcccaggaaatggtggtggtacagcactttccaaaggtgagccatcttctttatcagcatgaatcgagaaaactaaatctaaggagtgccccagtagtttaccagcttcttcaacaatctcgacatgattctcagcatagacaccaacatgatctcctgtctcataagttatccccgttctgctgatatcaaactccaaatgtatacagctcctatcagattcatgggtatgaagctctttttgtactgctacatcaactctgcaagggtggtgaatatcaatagtagtgttaccattggcgacatttgattccattgacttttgagttgtgaaacgtggatcgtgtgttacgactctatactcgggaataacggcagtatatggtgtagccacactcttatcatcttcgtcctttaaaagtttatccaattcactccacaacgattctttccacgcgttgaaatcatcctcaatgctttgatcgtcatcgccgagacccacctcaataagcctttttgctccttttttgcacagctcctcatctagaacgatcccgatcttattaaagtgttcgtattgtcggttgccaagggcaaacaccccgtatgcaagttgctgtaattttatgtcacgttcgttctcttcagtgaaccacttgtagaatctagcagcattgtctgtcggttctccatcgccgtatgtagctacacaaaaaaaagccaacgtctccttctttagtttttcctcgtattggtcgtcgtccgcagcgtaatcgtccaggtctataaccttgactgctgccttttcatatctggccttaatttcttccgacaatgctttagcaaagccttcagccgtacctgtctgggtgccaaaaaatatagagactcttgttttacctgaacctaggtctagatcatcatcttcatctttagccatcagactctttgggatcattagtggttttaattcaccagagcgatcagcagttgtttttttccacaaaagtacaacgaatccggctactaaggccaatgaagtagtcgcaatcactaaaaccacgtcatcgctcaaggagtcggttcccattatagatttcaactgcttaaacaaatcggatgcgtagagtgcggaggtcattttaattgtaagtcttgactagagcaagtgttatggtgaatttcaagtaattgaagtgctgggtctacagcttatatatgattttggcctgatgagcaacagaggctatcaccccgctgggataatgaaacttaccgattgcggcaggctaagcgaaaagtccaggccaatcgagggaagttaattgctcagctttcctgactaatcccaactttgaggggtgcatagtatggattcggtctcgtgcgagaagcttaaactttgttcaattccattagtagagggtatcatggaacttgcatgtgatgattacttcatatgatatcaggtactagcaatctcgattcgaatttattaggtaatgccgaccggaacaatagaaagacttcaagattgctaaaagaccaataagaacgtgtacagatgtcaggccatgagtttatttaagtgatctatgctacaattaaggttacaggagaaccgcattcggagttcgattatgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggattttttaactagctcagtgattttagcttcaattgcatggatatgcatgctaattagtcgagcgagaaaagttagcaaacttcccccaggtccatacggattacccataataggtaatatcctacagttggggcctaaaccccatcattctttagcaaaactctcacgaaagtatggacctgttatgtctcttaagttggggtccattacgacagttgtaattagtagtgcagaaacggctaagctcgtgcttcaaaaacatgacagctcattcagtaataggactgtactatctgcaataactgcactacgccaccacgaattctcggtaggatggcttccggtcggcaatcaatggaggaaattaagaaagatctgtaaagagcaaatgttttcggcaccacgcttagacgctagtgaaggtttaagaagggaaaaattacaaaaactacaggattatttagctgaaagatgtgaaagaggtacagctgttgatgtaggtgaggccgcctttacaacagctttgaatttgatgagcgcttctctattttccgccgaatttgttcaatttggctccgaaaccagccaggagatgaaagaggccatcagttcagtcgtaaagtcaataggtcgtcctaacctcgctgattattttccggttctgaaggccgttgacccccagggaatatacaaggaaacggaattgaatgtgggtcggctatttgtcaaattggatgaaatcatagatgaaaaattaagatccaggggtgagaagagagatttagtggaagtgttgcttgaaattcacaggagggacgaagcacaactttctagagacgatatcaggcatcttttgttggatttactggttgctggcaccgataccacctcaggaactatggaatgggccatgactgaactgattcgtaatccacagaagatggtcaagttgagaaatgagatcagatcattcatgtccgaatctggtaaacaacaaattgaggagtccgatatttcgtctttgtcttacttacaagctgtagttaaggaaaatttcagattgcatcctgcagcgccatttttggttccacataaggctaactgcgatgttgaaataaacggttatatcattccgaaaaatgcccaaatattcgtgaacgtctgggcatcggggagagactctaaaatctggaagaacccagatgaattcttgcctgaaagatttctgaacgaaaatgataacattgactttaaaggtagagactttgaactaatcccattcggttctggccgtcgtatgtgtccaggcttacctttagcggacagaatggtccaccaaatgttggttactttcgtcggaaattacgattggaagctggaaaacatgaaacctgaggagatggatatgaacgagaatttcggtattacactggaaaaagcgattcctttgaaagctattccaattaaagtataagtagatttggccactaacgggttagtagttgtgtaagtctattaaatttgatttttgtttatggatgatcatcgtagtggctatctgtttacctgtaggacatcctagggtgggatggtgatgtacaccccctcaatcttcagatgcaacactatgtggtaggtcattgacataaggtttaggaaagacctgttttttgaccaataaatggaacaggaaggaaaggaggaaccagtttacgaaccccgtcgatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 82 - DNA, 8019 bp, synthetic construct
  source 1..8019: mol_type = other DNA
aacttgccacagttcagcagaaagaatcagagttgaaagaattcataaacgatgtagaagaagaagatggtgtacctcaatatgccgcttcaagtgatgaagaaggcgacgaaaatgtgtcaaaaaagagaaaaatggagagcccaccaactgaaacagttgattctggcgaacagccaactaacccagtccaccctcagctttcctccatcttagagagtctatctagaacaactggactaacaccagaacctgcttctacttcggacgagtcagcaacagcaacgcagaagcaagatgaaacacctgtttcttcatctataaatcctgctctagccagtttgctgtccaaacttaacggttaattcttaatgtcttccccaatcacttgagtacgaactatgtattatataatcaggcatcttttctttttttcttttttttttgtctctcgcttggatcccaacaccatatttcagatctcctgatgactgactcactgataataaaaatacggcttcagaatttctcaagactacactcactgtccgacttcaagtgacccttgtgactgacactttgggagtccctattctacttagtctcatatcgcatgaaacttttgataaattattttctgataggaatttttcatcagatattatcatcgcggcttacgtaataacaaaaaaaattgatggagtctatactaggctaacataaactaagttattaattaaacaaaacaaaacgtactagcattactgtcatatataagggctcctaactaaaactgtaaagacttcccgtttaccatacgtctctgagatagcgaccttccgtttgtaacttcttaacgatggcttcagcttcggatgaagatacaccttcttgttcttgcactatcgtatgtaatgtacgatgaacatctctagccatacctttggcatcaccgcaaacatatagatatccttcctccttgatcaagtcccaaacttgcgcagccttctccatcattttatgctgaacatattccttttgtgctccctctctcgaaaaggccataattaattcggaaatcacgccctgatcgacgaaattgttaagctcgtcttcgtaaatgaaatccatttgcctgttccgacagccaaagaacagtaaactggagcctaattcttcaccgtcttctttcagcgccattctttcctgcaaaaaaccacggaaaggtgctagaccggttccgggacccaccataactataggtgtagacgggttcgagggcaacttaaaattagaagctcttataaagataggggcaccagaacattcgtgagacttttcagctgggactgcatttttcatccaagtggaacaaacacctttatggatcctacctgttggggttggaccataaaccaacgctgaagtgacatgaactctgctgggtgccaaccttggagaagaagaaatactgtaatatcttggttgcaatctcggggcaatggcagcgaaaaagactccaagtggaggtttggcagagggaaacgctgccatcacttctaataatgatctttgtgatgcaacaatccactgtgagtattcgtctttaccatcaggactggtgagatgttttaatttttctgcttcactaggttcagtggcatatgcagctaaagctactagtgctgattttcgtggcggattcaataaatctgcatacctagccagaccggttcctaaagtacaaggcccaggaaatggtggtggtacagcactttccaaaggtgagccatcttctttatcagcatgaatcgagaaaactaaatctaaggagtgccccagtagtttaccagcttcttcaacaatctcgacatgattctcagcatagacaccaacatgatctcctgtctcataagttatccccgttctgctgatatcaaactccaaatgtatacagctcctatcagattcatgggtatgaagctctttttgtactgctacatcaactctgcaagggtggtgaatatcaatagtagtgttaccattggcgacatttgattccattgacttttgagttgtgaaacgtggatcgtgtgttacgactctatactcgggaataacggcagtatatggtgtagccacactcttatcatcttcgtcctttaaaagtttatccaattcactccacaacgattctttccacgcgttgaaatcatcctcaatgctttgatcgtcatcgccgagacccacctcaataagcctttttgctccttttttgcacagctcctcatctagaacgatcccgatcttattaaagtgttcgtattgtcggttgccaagggcaaacaccccgtatgcaagttgctgtaattttatgtcacgttcgttctcttcagtgaaccacttgtagaatctagcagcattgtctgtcggttctccatcgccgtatgtagctacacaaaaaaaagccaacgtctccttctttagtttttcctcgtattggtcgtcgtccgcagcgtaatcgtccaggtctataaccttgactgctgccttttcatatctggccttaatttcttccgacaatgctttagcaaagccttcagccgtacctgtctgggtgccaaaaaatatagagactcttgttttacctgaacctaggtctagatcatcatcttcatctttagccatcagactctttgggatcattagtggttttaattcaccagagcgatcagcagttgtttttttccacaaaagtacaacgaatccggctactaaggccaatgaagtagtcgcaatcactaaaaccacgtcatcgctcaaggagtcggttcccattatagatttcaactgcttaaacaaatcggatgcgtagagtgcggaggtcattttaattgtaagtcttgactagagcaagtgttatggtgaatttcaagtaattgaagtgctgggtctacagcttatatatgattttggcctgatgagcaacagaggctatcaccccgctgggataatgaaacttaccgattgcggcaggctaagcgaaaagtccaggccaatcgagggaagttaattgctcagctttcctgactaatcccaactttgaggggtgcatagtatggattcggtctcgtgcgagaagcttaaactttgttcaattccattagtagagggtatcatggaacttgcatgtgatgattacttcatatgatatcaggtactagcaatctcgattcgaatttattaggtaatgccgaccggaacaatagaaagacttcaagattgctaaaagaccaataagaacgtgtacagatgtcaggccatgagtttatttaagtgatctatgctacaattaaggttacaggagaaccgcattcggagttcgattatgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggattttccgacttttctattggttgtattgtcaatcatttggattagtaccatgatcttgaccttcaatagtcgcgcccgcaagtcgtctaaattgccgccaggacctaatagattcccaattatcggaaacattttggagattggtccaaaaccgcataaatccttagctaaacttgcggcaaaatatggtccagtcatgtcattaaagttgggtacgttaacaacagtgatcatcagtagccctaaagcagctaaggctgtgttacaaactcatgatctaattttttcttcaagaaccgttccttgtgcagcagagattttatctcatcatgagtcgtcaatcgcctggttgccagccgaaaatagatggcgtaaattaagaaaactttgtcaagagcaaatgttttccgttgcaaggttagatgcctctcaaaatttacgacgagagaagctaagaaagcttaggggttacgttggtgcttgtagtgaaacagggagagcggtcgacataggggatgcagcatttactacggcgctaaacttaatgagtgctactttattttccttagaatttgcaccagaatttgactctgactcatcacaagaaatgaaagacgtggtctggggtgttatgaaatgcactgggagcccaaatttcgccgactacttccccattctaaaaagcgtagaccctcagggaatattaaaggaagctaagacgtatttccagaagctcttcgtaatatttgacggcattatagacgacaaattgaaaagcggtggagagtctggcaagcaggatttactcgaagcactgatagaaattaaccgtagggatgagaacgaattgacaaaaaatgatatcaagcacctgcttttagatttgttcgtagcaggtactgacaccacttctgtgacagttgaatgggctatggctgaattgcttaaacacccccataagatgtcccgtgtccggcaagaaatacgtgatactgtcggtgaaaacgtggaagttgaagaatccgatatttctagacttccatatctgcaagctgtagtgaaagagacatttaggctacacccagctgctccttttcttgtcccccacaaagttaatgctgatgttgaaataaaaggttttacagttccaaaaaacgcacaagttctggttaatgtatgggcttcgggcagggatcctgatacttggcccgaagccgaatcattttcccctgaaagattcatggatggaagacaaatagatattagaggaaaagattttgaattgattcctttcggtagtggcaggagaatatgtcctggtctgccgctagcctacagaatggtccatttgatgttggcgaatttagttgggtctttcgagtggagactcgatggcgattgcaaggaaatcgatatggatgacaaatttggtctgaccttgcagaaggctattccactaaaggccgtaccaatgaagttgtaagtagatttggccactaacgggttagtagttgtgtaagtctattaaatttgatttttgtttatggatgatcatcgtagtggctatctgtttacctgtaggacatcctagggtgggatggtgatgtacaccccctcaatcttcagatgcaacactatgtggtaggtcattgacataaggtttaggaaagacctgttttttgaccaataaatggaacaggaaggaaaggaggaaccagtttacgaaccccgtcgatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 83 - DNA, 1064 bp, synthetic construct
  source 1..1064: mol_type = other DNA
tttaattgtaagtcttgactagagcaagtgttatggtgaatttcaagtaattgaagtgctgggtctacagcttatatatgattttggcctgatgagcaacagaggctatcaccccgctgggataatgaaacttaccgattgcggcaggctaagcgaaaagtccaggccaatcgagggaagttaattgctcagctttcctgactaatcccaactttgaggggtgcatagtatggattcggtctcgtgcgagaagcttaaactttgttcaattccattagtagagggtatcatggaacttgcatgtgatgattacttcatatgatatcaggtactagcaatctcgattcgaatttattaggtaatgccgaccggaacaatagaaagacttcaagattgctaaaagaccaataagaacgtgtacagatgtcaggccatgagtttatttaagtgatctatgctacaattaaggttacaggagaaccgcattcggagttcgattatgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaaca

SEQ ID NO: 84 - DNA, 250 bp, synthetic construct
  source 1..250: mol_type = other DNA
acgggaagtctttacagttttagttaggagcccttatatatgacagtaatgctagtacgttttgttttgtttaattaataacttagtttatgttagcctagtatagactccatcaattttttttgttattacgtaagccgcgatgataatatctgatgaaaaattcctatcagaaaataatttatcaaaagtttcatgcgatatgagactaagtagaatagggactcccaaagtgtcagtcacaagggtc

SEQ ID NO: 85 - DNA, 250 bp, synthetic construct
  source 1..250: mol_type = other DNA
gtagatttggccactaacgggttagtagttgtgtaagtctattaaatttgatttttgtttatggatgatcatcgtagtggctatctgtttacctgtaggacatcctagggtgggatggtgatgtacaccccctcaatcttcagatgcaacactatgtggtaggtcattgacataaggtttaggaaagacctgttttttgaccaataaatggaacaggaaggaaaggaggaaccagtttacgaaccccgtc

SEQ ID NO: 86 - DNA, 560 bp, synthetic construct
  source 1..560: mol_type = other DNA
aacttgccacagttcagcagaaagaatcagagttgaaagaattcataaacgatgtagaagaagaagatggtgtacctcaatatgccgcttcaagtgatgaagaaggcgacgaaaatgtgtcaaaaaagagaaaaatggagagcccaccaactgaaacagttgattctggcgaacagccaactaacccagtccaccctcagctttcctccatcttagagagtctatctagaacaactggactaacaccagaacctgcttctacttcggacgagtcagcaacagcaacgcagaagcaagatgaaacacctgtttcttcatctataaatcctgctctagccagtttgctgtccaaacttaacggttaattcttaatgtcttccccaatcacttgagtacgaactatgtattatataatcaggcatcttttctttttttcttttttttttgtctctcgcttggatcccaacaccatatttcagatctcctgatgactgactcactgataataaaaatacggcttcagaatttctcaagactacactcactgtccgacttcaagt

(19) United States
(12) Patent Application Publication
(10) Pub. No.: US 2026/0265705 A1
(43) Pub. Date: Sep. 10, 2026

(54) RECOMBINANT POLYPEPTIDES FOR BIOSYNTHESIS OF THYMOHYDROQUINONE (THQ)

(71) Applicant: Epimeron USA, Inc., Sunnyvale, CA (US)

(72) Inventors: Trish Choudhary, Sunnyvale, CA (US); Matthew Workentine, Sunnyvale, CA (US); Christian Hobson, Sunnyvale, CA (US); Michal Pyc, Sunnyvale, CA (US)

(73) Assignee: Epimeron USA, Inc., Sunnyvale, CA (US)

(21) Appl. No.: 19/166,755

(22) PCT Filed: Mar. 19, 2024
(86) PCT No.: PCT/US2024/020520

§ 371 Date: Sep. 18, 2025

Related U.S. Application Data
(60) Provisional application No. 63/491,230, filed on Mar. 20, 2023.

(51) Int. Cl. C12N 9/02; C12P 7/22

(52) U.S. Cl. CPC C12N 9/0071; C12N 9/0042; C12P 7/22; C12Y 106/02004

(57) ABSTRACT

The present disclosure relates to recombinant polypeptides with cytochrome P450 (CYP) activity capable of selectively catalyzing the conversion of precursor compounds, carvacrol and/or thymol, to the product compound, thymohydroquinone (THQ), and recombinant host cells that heterologously express genes encoding these polypeptides (e.g., in yeast) and that can be used for enhanced fermentative bioproduction of the product compound THQ.

### CROSS REFERENCE TO RELATED APPLICATIONS

This application claims priority of U.S. Provisional Patent Application No. 63/491,230, filed Mar. 20, 2023, the entirety of which is hereby incorporated by reference herein.

### FIELD

The present disclosure relates to recombinant polypeptides with cytochrome P450 (CYP) activity capable of selectively catalyzing the conversion of precursor compounds thymol and carvacrol to the product compound, thymohydroquinone (THQ), and the engineering of these polypeptides for heterologous expression in yeast allowing for improved fermentative bioproduction of THQ.

### REFERENCE TO SEQUENCE LISTING

The official copy of the Sequence Listing is submitted concurrently with the specification via USPTO Patent Center as a WIPO Standard ST.26 formatted XML file with file name “21896-021WO1.xml”, a creation date of Mar. 18, 2024, and a size of 146,712 bytes. This Sequence Listing filed via USPTO Patent Center is part of the specification and is incorporated in its entirety by reference herein.

### BACKGROUND

Thymohydroquinone (THQ) is a phenolic terpene that is a minor component in many essential oils, such as oregano oil. THQ acts an antioxidant without disrupting the flavor profile of oregano oil. The THQ precursors, thymol and carvacrol, are abundant in oregano oil but have a strong taste and odor. We have developed a bioconversion process with a P450/CPR enzyme pair to convert thymol and carvacrol in oregano oil to THQ. Two cytochrome P450 enzymes from oregano, CYP76S40, and CYP736A300, have been identified that can conduct this reaction when expressed heterologously in Nicotiana benthamiana or Saccharomyces cerevisiae (see e.g., Krause, et al. “The Biosynthesis of Thymol, Carvacrol, and Thymohydroquinone in Lamiaceae Proceeds via Cytochrome P450s and a Short-Chain Dehydrogenase.” Proceedings of the National Academy of Sciences 118, no. 52 (Dec. 28, 2021). These P450 enzymes, however, are sub-optimal for commercial bioproduction of THQ in yeast.

Accordingly, there remains a need for a more efficient recombinant polypeptides, recombinant cell systems, and biocatalytic methods for the commercially viable biosynthetic production of THQ.

### SUMMARY

The present disclosure relates generally to recombinant polypeptides with cytochrome P450 (CYP) activity capable of selectively catalyzing the conversion of the precursor compounds, thymol and carvacrol, to the product compound, THQ, and the engineering of these polypeptides for heterologous expression in yeast allowing for improved fermentative bioproduction of these product compounds. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides recombinant host cell comprising a heterologous nucleic acid encoding a polypeptide with CYP activity capable of converting carvacrol and/or thymol to THQ, wherein the polypeptide comprises an amino acid sequence of at least 80% sequence identity to a sequence selected from the group consisting of SEQ ID NO: 8, 10, 12, 14, 16, 18, 20, 22, 24, 26, 28, 30, 32, 34, and 36. In at least one embodiment, the heterologous nucleic acid comprises: (a) a sequence of at least 80% identity to a sequence selected from the group consisting of SEQ ID NO: 7, 9, 11, 13, 15, 17, 19, 21, 23, 25, 27, 29, 31, 33, and 35; or (b) a codon degenerate sequence of a sequence selected from the group consisting of SEQ ID NO: 7, 9, 11, 13, 15, 17, 19, 21, 23, 25, 27, 29, 31, 33, and 35.

In at least one embodiment of the recombinant host cell of the present disclosure:
(a) the polypeptide with CYP activity comprises an amino acid sequence having at least 85%, at least 90%, at least 91%, at least 92%, at least 93%, at least 94%, at least 95%, at least 96%, at least 97%, at least 98%, at least 99%, or at least 99.5% sequence identity to a sequence selected from the group consisting of SEQ ID NO: 8, 10, 12, 14, 16, 18, 20, 22, 24, 26, 28, 30, 32, 34, and 36; or
(b) the polypeptide with CYP activity comprises an amino acid sequence selected from SEQ ID NO: 8, 10, 12, 14, 16, 18, 20, 22, 24, 26, 28, 30, 32, 34, and 36.

In at least one embodiment of the recombinant host cell of the present disclosure, the heterologous nucleic acid further comprises a sequence encoding a polypeptide with CPR activity; optionally, wherein the polypeptide with CPR activity comprises an amino acid sequence of at least 90%, at least 91%, at least 92%, at least 93%, at least 94%, at least 95%, at least 96%, at least 97%, at least 98%, at least 99%, or at least 99.5% sequence identity to SEQ ID NO: 6.

In at least one embodiment of the recombinant host cell of the present disclosure, the heterologous nucleic acid:
(a) is integrated at one or more sites in the host cell genome; optionally, it is integrated at two or more sites, or three or more sites; and/or
(b) is under the control of a promoter system selected from pGal1/10, and PCAT1:pFDH.

In at least one embodiment of the recombinant host cell of the present disclosure, the source organism of the host cell is selected from Saccharomyces cerevisiae, Pichia pastoris, Yarrowia lipolytica, and Escherichia coli.

In at least one embodiment of the recombinant host cell of the present disclosure, the recombinant host cell is Saccharomyces cerevisiae and the heterologous nucleic acid is integrated at one or more sites in the genome selected from X-2, X-4, XI-2, XII-4, NDE1, XII-5, Gal80, and ROQ1; optionally, the heterologous nucleic acid is integrated at three or more sites.

In at least one embodiment of the recombinant host cell of the present disclosure, the recombinant host cell is Pichia pastoris and the heterologous nucleic acid is integrated at one or more sites in the genome selected from AOX1, Int6, Int15, and HIS4; optionally, the heterologous nucleic acid is integrated at three or more sites.

In another aspect, the present disclosure provides a polynucleotide comprising a sequence of at least 80% identity to a sequence selected from the group consisting of SEQ ID NO: 7, 9, 11, 13, 15, 17, 19, 21, 23, 25, 27, 29, 31, 33, and 35.

In another aspect, the present disclosure provides an expression vector comprising the polynucleotide comprising a sequence of at least 80% identity to a sequence selected from the group consisting of SEQ ID NO: 7, 9, 11, 13, 15, 17, 19, 21, 23, 25, 27, 29, 31, 33, and 35. In at least one embodiment, the expression vector further comprises a polynucleotide sequence of at least 80% identity to SEQ ID NO: 5. In at least one embodiment, the expression vector can further comprise a control sequence; optionally, wherein the control sequence comprises a promoter system selected from pGal1/10, and pCAT1:pFDH1.

In another embodiment, the present disclosure provides a recombinant host cell comprising a polynucleotide comprising a sequence of at least 80% identity to a sequence selected from the group consisting of SEQ ID NO: 7, 9, 11, 13, 15, 17, 19, 21, 23, 25, 27, 29, 31, 33, and 35. In at least one embodiment, the host cell further comprises a polynucleotide sequence of at least 80% identity to SEQ ID NO: 5. In at least one embodiment, the host cell further comprises a control sequence; optionally, wherein the control sequence comprises a promoter system selected from pGal1/10, and pCAT1:pFDH1. In at least one embodiment, the source organism of the host cell is selected from Saccharomyces cerevisiae, Pichia pastoris, Yarrowia lipolytica, and Escherichia coli.

In at least one embodiment, the present disclosure provides a method for producing THQ comprising: (a) culturing a recombinant host cell of the present disclosure in a suitable medium comprising carvacrol and/or thymol; and (b) recovering the produced THQ.

In at least one embodiment, the present disclosure provides a method for preparing compound (1a) or a derivative of compound (1a)

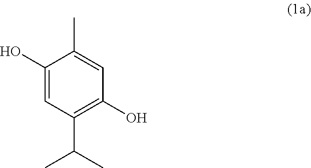

comprising contacting under suitable reactions conditions a recombinant polypeptide with CYP activity, wherein the polypeptide with CYP activity comprises an amino acid sequence of at least 80% sequence identity to a sequence selected from the group consisting of SEQ ID NO: 8, 10, 12, 14, 16, 18, 20, 22, 24, 26, 28, 30, 32, 34, and 36, with a compound (2a) and/or compound (2b)

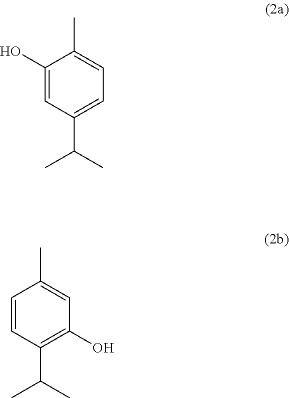

or a derivative of compound (2a) and/or a derivative of compound (2b). In at least one embodiment, the method further comprises contacting with a recombinant polypeptide with CPR activity; optionally, wherein the recombinant polypeptide with CPR activity comprises an amino acid sequence of at least 80% sequence identity to SEQ ID NO: 6.

In at least one embodiment of the method, a derivative of compound (1a) is prepared, wherein the derivative is selected from compound (1b), compound (1c), compound (1d), compound (1e), compound (1f), compound (1g), compound (1h), compound (1i), compound (1j), compound (1k), compound (1l), compound (1m), compound (1n), compound (1o), compound (1p), compound (1q), compound (1r), compound (1s), compound (1t), compound (1u), compound (1v), compound (1w), compound (1x), compound (1y), compound (1z), compound (1aa), compound (1ab), compound (1ac), compound (1ad), compound (1ae), compound (1af), compound (1ag), compound (1ah), and compound (1ai).

In at least one embodiment of the method, a derivative of compound (2a) and/or a derivative of compound (2b) is used to prepare a derivative of compound (1a), wherein the derivative of compound (2a) and/or a derivative of compound (2b), wherein the derivative is selected from compound (2c), compound (2d), compound (2e), compound (2f), compound (2g), compound (2h), compound (2i), compound (2j), compound (2k), compound (2l), compound (2m), compound (2n), compound (2o), compound (2p), and mixture thereof.

In at least one embodiment, the contacting with the recombinant polypeptides with CYP activity and CPR activity occurs in the presence of host cell that expresses the polypeptides. In at least one embodiment, the contacting with the recombinant polypeptides with CYP activity and CPR activity occurs in a cell-free system. In at least one embodiment, the method can further comprise a chemical step to form a derivative of compound (1a); optionally, wherein the derivative of compound (1a) is selected from compound (1b), compound (1c), compound (1d), compound (1e), compound (1f), compound (1g), compound (1h), compound (1i), compound (1j), compound (1k), compound (1l), compound (1m), compound (1n), compound (1o), compound (1p), compound (1q), compound (1r), compound (1s), compound (1t), compound (1u), compound (1v), compound (1w), compound (1x), compound (1y), compound (1z), compound (1aa), compound (1ab), compound (1ac), compound (1ad), compound (1ae), compound (1af), compound (1ag), compound (1ah), and compound (1ai).

In at least one embodiment, the disclosure provides an enzymatic reaction mixture composition, wherein the composition comprises: (a) a recombinant polypeptide with CYP activity comprising an amino acid sequence of at least 80% sequence identity to a sequence selected from the group consisting of SEQ ID NO: 8, 10, 12, 14, 16, 18, 20, 22, 24, 26, 28, 30, 32, 34, and 36; and (b) compound (2a) and/or compound (2b), or a derivative of compound (2a) and/or a derivative of compound (2b). In at least one embodiment, the composition further comprises a recombinant polypeptide with CPR activity; optionally, a polypeptide comprising an amino acid sequence of at least 80% sequence identity to SEQ ID NO: 6. In at least one embodiment, the composition comprises a derivative of compound (2a) and/or a derivative of compound (2b), wherein the derivative is selected from compound (2c), compound (2d), compound (2e), compound (2f), compound (2g), compound (2h), compound (2i), compound (2j), compound (2k), compound (2l), compound (2m), compound (2n), compound (2o), compound (2p), and mixture thereof.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIGS. 1A, 1B, and 1C show schematic depictions of the CYP and CPR gene insertion sites in the strain constructs described in Examples 1 and 2. FIG. 1A depicts the X-4 insertion site of the OG002 strain containing m-Venus and the URA3 gene under the bidirectional pGal10/1 promoter. FIG. 1B shows the X-4 insertion site of the OG003 strain containing CPR006 (SEQ ID NO: 6) and the URA3 gene (which was used as a landing site to integrate the two CYP genes) under the bidirectional pGal10/1 promoter. FIG. 1C shows the X-4 insertion site of the OG014 strain containing a non-functional CPR006 and the URA3 gene (which was used as a landing site to integrate various CYP homolog genes) under the bidirectional pGal10/1 promoter.

FIG. 2 depicts exemplary UHPLC profiles obtained for the recombinant host strains OG004, OG005, OG008 and OG009, which were constructed and screened for THQ production as described in Example 1.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### Definitions

“THQ” refers to the compound thymohydroquinone having the chemical structure shown as compound (1a) in Table 1 (below).

“THQ derivatives” as referenced in the present disclosure include, but are not limited to, structural analogs of THQ, such as thymoquinone (TQ), dithymoquinone (DTQ), and the other exemplary compounds shown below in Table 1 (below).

TABLE 1
Exemplary THQ and THQ derivative compounds

| Compound Name | Chemical Structure | |
|---|---|---|
| Thymohydroquinone (“THQ”) | 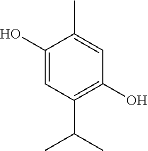 | (1a) |
| Thymoquinone (“TQ”) | 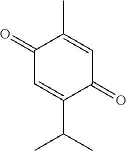 | (1b) |
| Dithymoquinone (“DTQ”) | 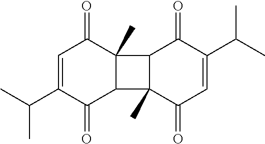 [SMILES: CC(C)C1=CC(=O)[C@]2(C)C(C1=O)[C@]1(C)C(=O)C=C(C(C)C)C(=O)C21] | (1c) |
| p-Methoxythymol | 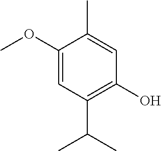 | (1d) |
| p-Methoxycarvacrol | 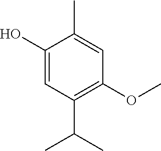 | (1e) |
| Thymohydroquinone dimethyl ether | 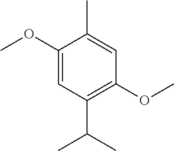 | (1f) |
| p-Acetylthymol | 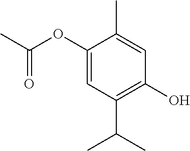 | (1g) |
| p-Acetylcarvacrol | 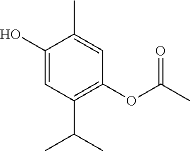 | (1h) |
| Di-O- acetylthymohydroquinone | 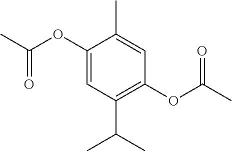 [SMILES: CC(=O)Oc1cc(C(C)C)c(OC(C)=O)cc1C] | (1i) |
| 2-hydroxy-5-[2-(pyrrolidin-1- yl)ethoxy]-p-cymene hydrochloride | 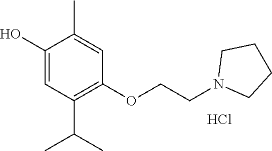 [SMILES: Cc1cc(OCCN2CCCC2)c(C(C)C)cc1O ; Cl] | (1j) |
| 5-isopropyl-2-methyl-4-(2- piperidino-ethoxy)-phenol | 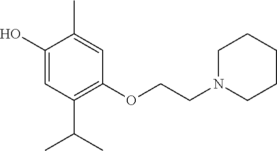 [SMILES: Cc1cc(OCCN2CCCCC2)c(C(C)C)cc1O] | (1k) |
| 5-isopropyl-2-methyl-4-(2- morpholino-ethoxy)-phenol | 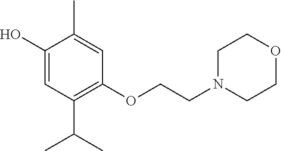 [SMILES: Cc1cc(OCCN2CCOCC2)c(C(C)C)cc1O] | (1l) |
| desacetylmoxisylyte | 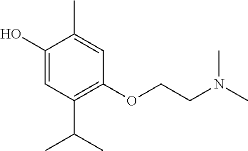 [SMILES: Cc1cc(OCCN(C)C)c(C(C)C)cc1O] | (1m) |
| moxisylyte | 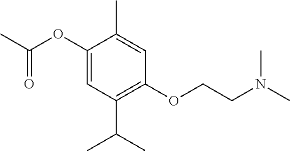 [SMILES: CC(=O)Oc1cc(C(C)C)c(OCCN(C)C)cc1C] | (1n) |
| moxisylyte hydrochloride | 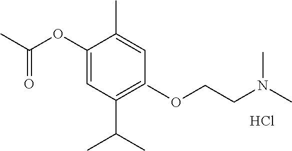 | (1o) |
| Desacetylmoxisylyte sulphate | 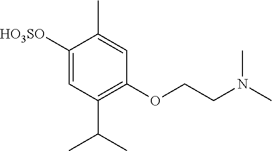 | (1p) |
| 5-(2-Methylaminoethoxy) carvacrol | 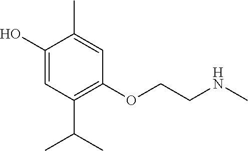 [SMILES: CNCCOc1cc(C)c(O)cc1C(C)C] | (1q) |
| 5-(2-Methylaminoethoxy) carvacrol hydrochloride | 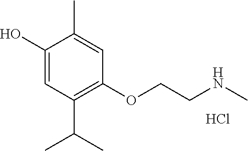 [SMILES: CNCCOc1cc(C)c(O)cc1C(C)C ; Cl] | (1r) |
| Monodesmethyl- desacetylmoxisylyte sulphate | 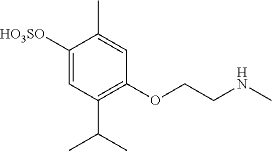 | (1s) |
| 5-(2-isopropyl-4-methoxy-5- methyl-phenoxy)-pyrimidine- 2,4-diamine | 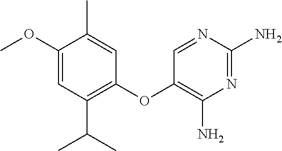 [SMILES: COc1cc(C(C)C)c(Oc2cnc(N)nc2N)cc1C] | (1t) |
| 5-(2-isopropyl-4-methoxy-5- methyl-phenoxy)-N-4- phenylpyrimidine-2,4- diamine | 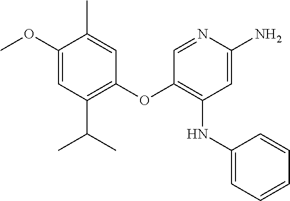 [SMILES: COc1cc(C(C)C)c(Oc2cnc(N)cc2Nc2ccccc2)cc1C] | (1u) |
| 2-amino-5-(2-isopropyl-4- methoxy-5-methyl-phenoxy)- 3H-pyrimidine-4-one | 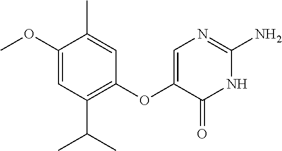 [SMILES: COc1cc(C(C)C)c(Oc2cnc(N)[nH]c2=O)cc1C] | (1v) |
| 4-chloro-5-(2-isopropyl-4- methoxy-5-methyl-phenoxy)- pyrimidin-2-ylamine | 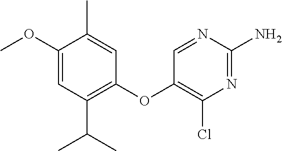 [SMILES: COc1cc(C(C)C)c(Oc2cnc(N)nc2Cl)cc1C] | (1w) |
| 6-methoxylthymol isobutyrate | 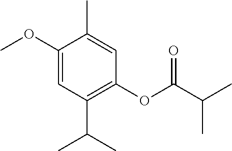 [SMILES: COc1cc(C(C)C)c(OC(=O)C(C)C)cc1C] | (1x) |
| thymoquinol 5-O-ß- glucopyranoside | 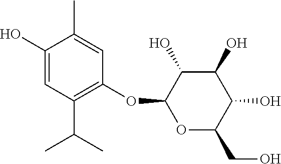 [SMILES: Cc1cc(O[C@@H]2O[C@H](CO)[C@@H](O)[C@H](O)[C@H]2O)c(C(C)C)cc1O] | (1y) |
| thymoquinol 2-O-ß- glucopyranoside | 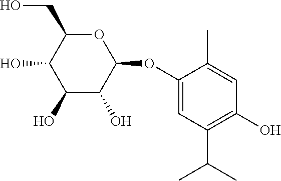 [SMILES: Cc1cc(O)c(C(C)C)cc1O[C@@H]1O[C@H](CO)[C@@H](O)[C@H](O)[C@H]1O] | (1z) |
| thymoquinol-2,5-O-ß- diglucopyranoside | 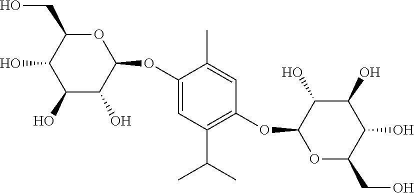 [SMILES: Cc1cc(O[C@@H]2O[C@H](CO)[C@@H](O)[C@H](O)[C@H]2O)c(C(C)C)cc1O[C@@H]1O[C@H](CO)[C@@H](O)[C@H](O)[C@H]1O] | (1aa) |
| 1-O-(4-methoxy-2-methyl-5- (1-methylethylphenyl)-ß-D- glucopyranoside | 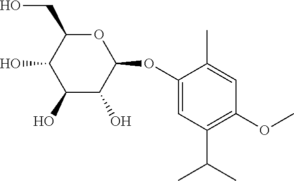 [SMILES: COc1cc(C)c(O[C@@H]2O[C@H](CO)[C@@H](O)[C@H](O)[C@H]2O)cc1C(C)C] | (1ab) |
| 1-O-(4-acetyl-2-methyl-5-(1- methylethylphenyl)-ß-D- glucopyranoside | 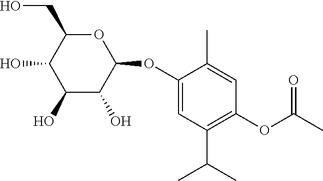 [SMILES: CC(=O)Oc1cc(C)c(O[C@@H]2O[C@H](CO)[C@@H](O)[C@H](O)[C@H]2O)cc1C(C)C] | (1ac) |
| Heyderiol | 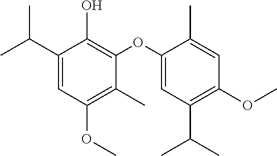 [SMILES: COc1cc(C)c(Oc2c(C)c(OC)cc(C(C)C)c2O)cc1C(C)C] | (1ad) |
| N-(3-hydroxy-6-methoxy- 2,4,5-trimethylbenzyl)-2-(4- hydroxy-2-isopropyl-5- methylphenoxy)ethylamine fumarate | 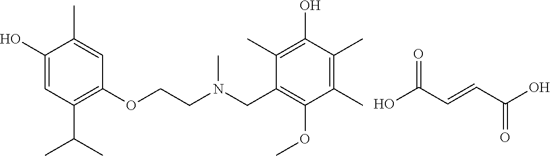 | (1ae) |
| N-(3-hydroxy-6-methoxy- 2,4,5-trimethylbenzyl)-2-(4- hydroxy-2-isopropyl-5- methylphenoxy)ethylamine | 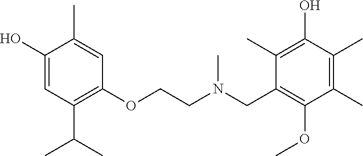 [SMILES: COc1c(C)c(C)c(O)c(C)c1CN(C)CCOc1cc(C)c(O)cc1C(C)C] | (1af) |
| 5-isopropyl-2-methyl-4- trifluoromethoxyphenol | 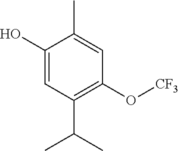 [SMILES: Cc1cc(OC(F)(F)F)c(C(C)C)cc1O] | (1ag) |
| querglanin | 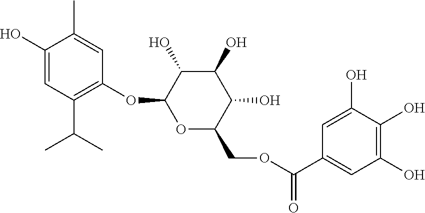 [SMILES: Cc1cc(O[C@@H]2O[C@H](COC(=O)c3cc(O)c(O)c(O)c3)[C@@H](O)[C@H](O)[C@H]2O)c(C(C)C)cc1O] | (1ah) |
| isoquerglanin | 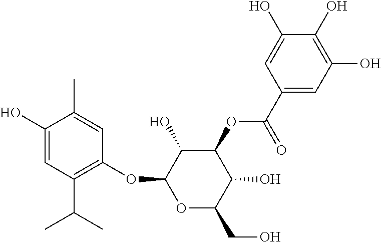 [SMILES: Cc1cc(O[C@@H]2O[C@H](CO)[C@@H](O)[C@H](OC(=O)c3cc(O)c(O)c(O)c3)[C@H]2O)c(C(C)C)cc1O] | (1ai) |

“THQ precursor” as used herein refers to a compound capable of being converted into THQ, or a THQ derivative, by an enzyme capable producing THQ alone, or in combination with another enzyme or a non-enzymatic chemical reaction. THQ precursors as referenced in the present disclosure include, but are not limited to, the exemplary compounds summarized in Table 2 (below).

TABLE 2
Exemplary THQ precursor compounds

| Compound Name | Chemical Structure | |
|---|---|---|
| Carvacrol | 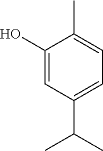 | (2a) |
| Thymol | 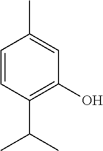 | (2b) |
| 5-[2-(pyrrolidin-1-yl)ethoxy]- p-cymene hydrochloride | 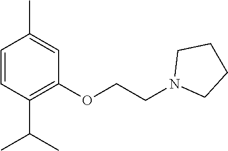 [SMILES: Cc1ccc(C(C)C)c(OCCN2CCCC2)c1] | (2c) |
| 5-isopropyl-2-methyl-4-(2- piperidino-ethoxy)-benzene | 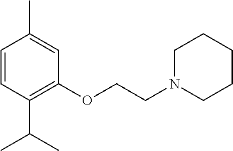 [SMILES: Cc1ccc(C(C)C)c(OCCN2CCCCC2)c1] | (2d) |
| 5-isopropyl-2-methyl-4-(2- morpholino-ethoxy)-benzene | 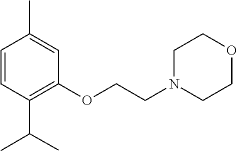 [SMILES: Cc1ccc(C(C)C)c(OCCN2CCOCC2)c1] | (2e) |
| 5-isopropyl-2-methyl-4-(2- dimethylamino-ethoxy)- benzene | 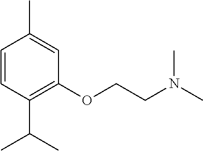 [SMILES: Cc1ccc(C(C)C)c(OCCN(C)C)c1] | (2f) |
| 5-isopropyl-2-methyl-4-(2- methylamino-ethoxy)- benzene | 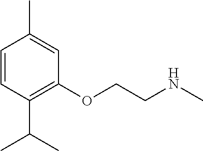 | (2g) |
| 5-isopropyl-2-methyl-4- (pyrimidine-2,4-diamine)- benzene | 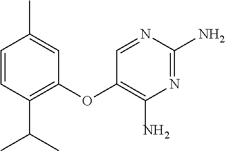 | (2h) |
| 5-isopropyl-2-methyl-4-(N-4- phenylpyrimidine-2,4- diamine)-benzene | 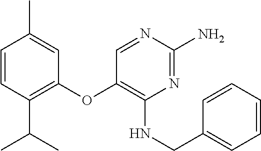 [SMILES: Cc1ccc(C(C)C)c(Oc2cnc(N)nc2NCc2ccccc2)c1] | (2i) |
| 5-isopropyl-2-methyl-4-(3H- pyrimidine-4-one)-benzene | 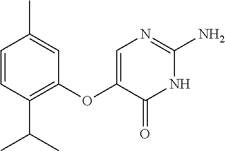 [SMILES: Cc1ccc(C(C)C)c(Oc2cnc(N)[nH]c2=O)c1] | (2j) |
| 5-isopropyl-2-methyl-4-(2- amino-4-chloropyrimidine)- benzene | 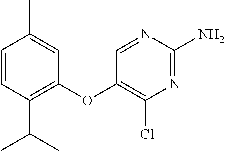 [SMILES: Cc1ccc(C(C)C)c(Oc2cnc(N)nc2Cl)c1] | (2k) |
| thymol isobutryate | 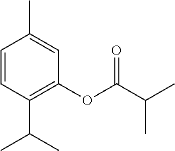 [SMILES: Cc1ccc(C(C)C)c(OC(=O)C(C)C)c1] | (2l) |
| thymol 5-O-ß- glucopyranoside | 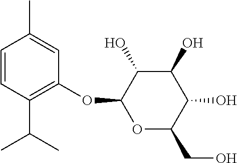 [SMILES: Cc1ccc(C(C)C)c(O[C@@H]2O[C@H](CO)[C@@H](O)[C@H](O)[C@H]2O)c1] | (2m) |
| carvacrol 2-O-ß- glucopyranoside | 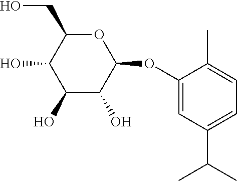 [SMILES: Cc1ccc(C(C)C)cc1O[C@@H]1O[C@H](CO)[C@@H](O)[C@H](O)[C@H]1O] | (2n) |
| N-(3-hydroxy-6-methoxy- 2,4,5-trimethylbenzyl)-2-(2- isopropyl-5- methylphenoxy)ethylamine | 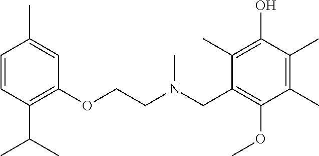 [SMILES: COc1c(C)c(C)c(O)c(C)c1CN(C)CCOc1cc(C)ccc1C(C)C] | (2o) |
| O-trifluoromethyl thymol | 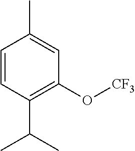 | (2p) |

“CYP activity” as used herein refers to the catalytic activity of a cytochrome P450 monooxygenase enzyme.

“CPR activity” as used herein refers to the catalytic activity of a cytochrome P450 reductase enzyme.

“Conversion” as used herein refers to the enzymatic conversion of a substrate(s) to a corresponding product(s). “Percent conversion” refers to the percent of the substrate that is converted to the product within a period of time under specified conditions. Thus, the “enzymatic activity” or “activity” of an enzymatic conversion can be expressed as “percent conversion” of the substrate to the product.

“Substrate” as used herein in the context of an enzyme mediated process refers to the compound or molecule acted on by the enzyme.

“Product” as used herein in the context of an enzyme mediated process refers to the compound or molecule resulting from the activity of the enzyme.

“Host cell” as used herein refers to a cell capable of being functionally modified with recombinant nucleic acids and functioning to express recombinant products, including polypeptides and compounds produced by activity of the polypeptides.

“Nucleic acid,” or “polynucleotide” as used herein interchangeably to refer to two or more nucleosides that are covalently linked together. The nucleic acid may be wholly comprised ribonucleosides (e.g., RNA), wholly comprised of 2′-deoxyribonucleotides (e.g., DNA) or mixtures of ribo- and 2′-deoxyribonucleosides. The nucleoside units of the nucleic acid can be linked together via phosphodiester linkages (e.g., as in naturally occurring nucleic acids), or the nucleic acid can include one or more non-natural linkages (e.g., phosphorothioester linkage). Nucleic acid or polynucleotide is intended to include single-stranded or double-stranded molecules, or molecules having both single-stranded regions and double-stranded regions. Nucleic acid or polynucleotide is intended to include molecules composed of the naturally occurring nucleobases (i.e., adenine, guanine, uracil, thymine, and cytosine), or molecules comprising that include one or more modified and/or synthetic nucleobases, such as, for example, inosine, xanthine, hypoxanthine, etc.

“Protein,” “polypeptide,” and “peptide” are used herein interchangeably to denote a polymer of at least two amino acids covalently linked by an amide bond, regardless of length or post-translational modification (e.g., glycosylation, phosphorylation, lipidation, myristilation, ubiquitination, etc.). As used herein “protein” or “polypeptide” or “peptide” polymer can include D- and L-amino acids, and mixtures of D- and L-amino acids.

“Naturally-occurring” or “wild-type” as used herein refers to the form as found in nature. For example, a naturally occurring nucleic acid sequence is the sequence present in an organism that can be isolated from a source in nature, and which has not been intentionally modified by human manipulation.

“Recombinant,” “engineered,” or “non-naturally occurring” when used herein with reference to, e.g., a cell, nucleic acid, or polypeptide, refers to a material, or a material corresponding to the natural or native form of the material, that has been modified in a manner that would not otherwise exist in nature, or is identical thereto but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Nucleic acid derived from” as used herein refers to a nucleic acid having a sequence at least substantially identical to a sequence of found in naturally in an organism. For example, cDNA molecules prepared by reverse transcription of mRNA isolated from an organism, or nucleic acid molecules prepared synthetically to have a sequence at least substantially identical to, or which hybridizes to a sequence at least substantially identical to a nucleic sequence found in an organism.

“Coding sequence” refers to that portion of a nucleic acid (e.g., a gene) that encodes an amino acid sequence of a protein.

“Heterologous nucleic acid” as used herein refers to any polynucleotide that is introduced into a host cell by laboratory techniques and includes polynucleotides that are removed from a host cell, subjected to laboratory manipulation, and then reintroduced into a host cell.

“Codon degenerate” describes a nucleotide sequence that has one or more different codons relative to the reference nucleotide sequence, but which encodes a polypeptide that is identical to the polypeptide encoded by a reference nucleotide sequence. The different codons between the nucleotide sequence and the reference nucleotide sequence are called “synonyms” or “synonymous” codons in that they use different triplets of nucleotides to encode the same amino acid in a polypeptide.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several different “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the imine reductase enzymes may be codon optimized for optimal production from the host organism selected for expression.

“Preferred, optimal, high codon usage bias codons” refers to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; CodonW, John Peden, University of Nottingham; McInerney, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” as used herein refers to all sequences, which are necessary or advantageous for the expression of a polynucleotide and/or polypeptide as used in the present disclosure. Each control sequence may be native or foreign to the nucleic acid sequence encoding a polypeptide. Such control sequences include, but are not limited to, a leader, a promoter, a polyadenylation sequence, a pro-peptide sequence, a signal peptide sequence, and a transcription terminator. At a minimum, control sequences typically include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” as used herein refers to a configuration in which a control sequence is appropriately placed (e.g., in a functional relationship) at a position relative to a polynucleotide sequence or polypeptide sequence of interest such that the control sequence directs or regulates the expression of the sequence of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Percentage of sequence identity,” “percent sequence identity,” “percentage homology,” or “percent homology” are used interchangeably herein to refer to values quantifying comparisons of the sequences of polynucleotides or polypeptides, and are determined by comparing two optimally aligned sequences over a comparison window, wherein the portion of the polynucleotide or polypeptide sequence in the comparison window may comprise additions or deletions (or gaps) as compared to the reference sequence for optimal alignment of the two sequences. The percentage values may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215:403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length nucleic acid or polypeptide sequence. A reference sequence typically is at least 20 nucleotide or amino acid residue units in length but can also be the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (or gaps) of 20 percent or less as compared to the reference sequence (which does not comprise additions or deletions) for optimal alignment of the two sequences.

“Substantial identity” or “substantially identical” refers to a polynucleotide or polypeptide sequence that has at least 70% sequence identity, at least 80% sequence identity, at least 85% sequence identity, at least 90% sequence identity, at least 95% sequence identity, or at least 99% sequence identity, as compared to a reference sequence over a comparison window of at least 20 nucleoside or amino acid residue positions, frequently over a window of at least 30-50 positions, wherein the percentage of sequence identity is calculated by comparing the reference sequence to a sequence that includes deletions or additions which total 20 percent or less of the reference sequence over the window of comparison.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered imine reductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Isolated” as used herein in reference to a molecule means that the molecule (e.g., THQ, polynucleotide, polypeptide) is substantially separated from other compounds that naturally accompany it, e.g., protein, lipids, and polynucleotides. The term embraces nucleic acids which have been removed or purified from their naturally occurring environment or expression system (e.g., host cell).

“Substantially pure” refers to a composition in which a desired molecule is the predominant species present (i.e., on a molar or weight basis it is more abundant than any other individual macromolecular species in the composition) and is generally a substantially purified composition when the object species comprises at least about 50 percent of the macromolecular species present by mole or % weight.

“Recovered” as used herein in relation to an enzyme, protein, or compound (e.g., THQ), refers to a more or less pure form of the enzyme, protein, or compound.

Homologous Genes Encoding Recombinant Polypeptides with CYP Activity

The present disclosure provides genes encoding polypeptides with cytochrome P450 (CYP) monooxygenase activity capable of converting the substrate compounds, carvacrol and/or thymol to the product compound, THQ, as illustrated by the reaction of Scheme 1 (below).

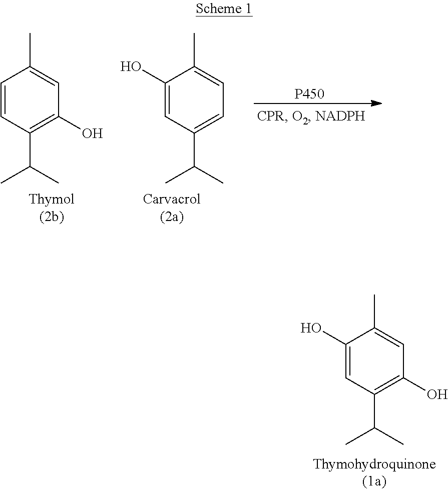

As shown in Scheme 1, the conversion of carvacrol (compound 2a) and/or thymol (compound 2b) to the product compound, THQ (compound 1a) can be catalyzed by the CYP polypeptide of the present disclosure in the presence of a cytochrome P450 reductase (CPR) enzyme, the cofactor NADPH, and oxygen. As described herein, CYP polypeptides with the activity of Scheme 1 were identified by sequence homology analysis of two cytochrome P450 enzymes from oregano (Origanum vulgare), CYP76S40, and CYP736A300. These two CYP polypeptides have been previously characterized as having this activity and heterologous genes encoding have been shown to be capable of expressing this activity in Nicotiana benthamiana or Saccharomyces cerevisiae (see e.g., Krause, et al. “The Biosynthesis of Thymol, Carvacrol, and Thymohydroquinone in Lamiaceae Proceeds via Cytochrome P450s and a Short-Chain Dehydrogenase.” Proceedings of the National Academy of Sciences 118, no. 52 (Dec. 28, 2021). CYP76S40 has the following 491 amino acid sequences:

(SEQ ID NO: 2; CYP002)
MDFLTPCLVVASIAWICMLILRARKPSKLPPGPYGPPIIGNILHLGPKPH
RSLADLARKYGPVMKLRLGSVTTVVISSPEAAKAVLQKHDSSFWNRPAPS
SVRAVGHDEFSVAWLPVDKQWRKLRKIMKELMFSSPRLDAGQGMRRAKLQ
QLSDYVWGRCQAGRAVEVGEAAFTTSLNLMSATLFSTDFARFDSDSSQEM
KEVVWGVMKCVGSPNLVDYFPVLKSLDPQGILKDAKFCFGKLFAIFDEIL
DERLKISRGEKQDLVEALIDLNQRDVPQLSRDDINHLLLDLFVAGSDTTS
GTVEWAMTELIRHPEKMTKLRNEITSFVEENGPIEESDISRLPYLQAVVK
ETFRLHPVAPFLLPHKASSDIEINGYTVPKNAQILVNIWASGRDPINWVD
ADKFVPERFLSENKGLNFIGQDFELIPFGAGRRICPGLPLANRMVHQMLV
TFVGNFEWKLEGIKVEEMDMDENFGLTLQKAIPLRAIPTKL

CYP736A300 has the following 498 amino acid sequence:

(SEQ ID NO: 4; CYP003)
MEWFWAALSLIVFLSLLHQLLKKEKKREANLPPSPIALPVIGHLHLLGKN
LPLKLHAIAERHGPIVFLRLGLVRALVVSTAAGAELVLKTHDLVFSGRVH
HQASRYLGYDQKNIVFAPYGAYWRNMRRLCMVKLLNAAKINEFRPVRRAE
LEETVASMRRAAEERGVVDVSAVISGVIGDMNSLMVFGRKYVDRDLDEEL
GFKAVIDEMLHVGALPNLGDFFPFMAALDLQGLDRRMKELSKIFDGFLER
IIDDHLLKKTENTKKGDFVDTMLAVMEAGEADFEFDRRHVKAVLLDMLIA
GMDTSASTVEWALSELIRHPEITKKLQKELEQVVGMDQMVDESHLDKLDY
LDSVLKETLRLYPPGRLLVHETMEECTVNGFHIPKGTWTFVNMWSIGRDP
AMWHEPEKFVPERFAGENLDFLGQNFKFIPFGAGRRSCPGLQLGLTFVRL
VLAQLVHCFDWELPNGMVPSDLDMNEKFGIVTSRDKHLMAIPTYRLNK

As described in the Examples and elsewhere herein, the CYP76S40 and CYP736A300 sequences were used to identify 128 unique homologs with at least 60% sequence identity from 45 different plant species. These candidate homolog polypeptides were screened for heterologous expression of the desired activity of Scheme 1 in yeast systems. In a first screening system, a heterologous CPR gene from Arabidopsis thaliana encoding the 692 amino acid CPR polypeptide of SEQ ID NO: 6 (CPR006) was also integrated into the yeast genome to provide the necessary P450 reductase activity.

(SEQ ID NO: 6; CPR006)
MTSALYASDLFKQLKSIMGTDSLSDDVVLVIATTSLALVAGFVVLLWKKT
TADRSGELKPLMIPKSLMAKDEDDDLDLGSGKTRVSIFFGTQTGTAEGFA
KALSEEIKARYEKAAVKVIDLDDYAADDDQYEEKLKKETLAFFCVATYGD
GEPTDNAARFYKWFTEENERDIKLQQLAYGVFALGNRQYEHFNKIGIVLD
EELCKKGAKRLIEVGLGDDDQSIEDDFNAWKESLWSELDKLLKDEDDKSV
ATPYTAVIPEYRVVTHDPRFTTQKSMESNVANGNTTIDIHHPCRVDVAVQ
KELHTHESDRSCIHLEFDISRTGITYETGDHVGVYAENHVEIVEEAGKLL
GHSLDLVFSIHADKEDGSPLESAVPPPFPGPCTLGTGLARYADLLNPPRK
SALVALAAYATEPSEAEKLKHLTSPDGKDEYSQWIVASQRSLLEVMAAFP
SAKPPLGVFFAAIAPRLQPRYYSISSSPRLAPSRVHVTSALVYGPTPTGR
IHKGVCSTWMKNAVPAEKSHECSGAPIFIRASNFKLPSNPSTPIVMVGPG
TGLAPFRGFLQERMALKEDGEELGSSLLFFGCRNRQMDFIYEDELNNFVD
QGVISELIMAFSREGAQKEYVQHKMMEKAAQVWDLIKEEGYLYVCGDAKG
MARDVHRTLHTIVQEQEGVSSSEAEAIVKKLQTEGRYLRDVW.

In a second screening system, the heterologous CPR of SEQ ID NO: 6 was knocked out and the native yeast cytochrome P450 reductase activity was relied upon to provide the reductase activity for the reaction.

In at least one embodiment of the present disclosure, the CYP76S40 polypeptide of SEQ ID NO: 2 can be encoded for heterologous expression by the yeast codon-optimized nucleotide sequence of SEQ ID NO: 1.

(SEQ ID NO: 1)
ATGGATTTCTTAACCCCCTGTCTCGTGGTAGCATCCATTGCCTGGATTTG
TATGTTGATACTAAGAGCAAGAAAACCGTCTAAACTTCCTCCTGGCCCAT
ATGGTCCGCCCATCATAGGTAATATACTACATTTAGGTCCTAAACCTCAT
AGATCTTTGGCCGACTTGGCCCGAAAATATGGGCCAGTCATGAAGCTTAG
GTTGGGTTCGGTAACCACAGTTGTTATATCTTCCCCAGAAGCAGCTAAAG
CTGTTTTACAAAAACACGATTCATCATTTTGGAATAGACCTGCTCCTTCT
TCTGTTAGGGCGGTTGGTCACGATGAATTCTCAGTCGCTTGGCTGCCTGT
AGACAAACAATGGAGAAAACTTCGGAAAATAATGAAAGAATTAATGTTCT
CATCGCCAAGATTAGATGCCGGCCAAGGTATGAGACGTGCAAAACTTCAA
CAATTAAGTGATTATGTCTGGGGTAGGTGCCAGGCTGGTAGAGCGGTCGA
GGTTGGAGAGGCTGCATTCACGACTTCTCTGAATTTAATGTCAGCTACAT
TATTTTCTACTGATTTTGCTCGTTTCGATTCCGATAGCAGTCAGGAAATG
AAGGAAGTGGTATGGGGCGTTATGAAGTGTGTGGGTTCTCCAAACTTGGT
AGATTACTTTCCAGTCTTGAAATCCCTCGATCCGCAAGGGATCCTAAAAG
ACGCAAAGTTCTGCTTTGGGAAGTTGTTCGCGATCTTTGACGAAATTCTG
GATGAACGCTTGAAGATCTCTCGTGGTGAAAAGCAGGATTTGGTAGAGGC
ACTAATCGATTTAAATCAACGTGACGTTCCTCAACTAAGCCGCGACGATA
TTAACCACCTATTGTTAGACTTATTTGTTGCTGGTTCAGATACTACTAGT
GGTACAGTCGAATGGGCAATGACAGAATTGATTAGGCATCCCGAAAAGAT
GACTAAGTTGAGAAATGAGATTACTTCATTTGTTGAAGAGAACGGCCCAA
TTGAAGAAAGTGATATCAGCAGATTGCCATACTTACAAGCCGTCGTTAAA
GAGACGTTTAGACTTCATCCTGTGGCCCCATTCCTACTTCCACATAAGGC
TAGCTCGGACATTGAAATTAACGGTTACACGGTTCCTAAAAATGCACAGA
TCCTGGTTAACATTTGGGCCTCCGGAAGGGATCCGATTAACTGGGTGGAT
GCTGATAAGTTTGTGCCAGAAAGGTTTTTAAGTGAGAATAAAGGACTGAA
CTTTATAGGACAGGATTTTGAACTCATACCCTTTGGCGCTGGTAGAAGAA
TTTGTCCAGGGTTGCCCTTGGCTAATAGAATGGTCCATCAAATGCTGGTG
ACCTTCGTTGGAAATTTCGAGTGGAAGCTTGAAGGAATAAAGGTAGAGGA
AATGGACATGGACGAAAATTTTGGCTTAACCTTGCAAAAAGCAATCCCAC
TACGAGCGATTCCAACAAAATTATAA

In at least one embodiment of the present disclosure, the CYP736A300 polypeptide of SEQ ID NO: 4 can be encoded for heterologous expression by the yeast codon-optimized nucleotide sequence of SEQ ID NO: 3.

(SEQ ID NO: 4)
ATGGAGTGGTTTTGGGCGGCTCTCTCATTGATAGTCTTTTTGTCGTTGTT
ACACCAATTACTAAAAAAGGAGAAGAAAAGGGAAGCTAACCTCCCACCGA
GTCCTATTGCTCTTCCTGTAATCGGCCATTTACACCTATTGGGAAAAAAC
CTTCCCTTAAAGCTGCACGCAATTGCCGAACGTCATGGTCCTATAGTTTT
TTTAAGATTAGGCCTTGTACGTGCCTTGGTGGTTAGCACAGCAGCTGGGG
CGGAGTTAGTGTTAAAAACTCATGATTTGGTTTTTAGTGGAAGAGTTCAT
CATCAAGCATCTCGCTACCTTGGTTATGACCAAAAGAATATCGTATTTGC
ACCTTATGGGGCATATTGGCGAAACATGCGTAGATTATGTATGGTCAAAT
TGTTGAATGCTGCCAAGATAAATGAATTTAGGCCCGTAAGAAGAGCTGAA
TTAGAAGAAACCGTTGCTTCCATGAGAAGGGCAGCTGAAGAAAGGGGAGT
GGTGGACGTGTCAGCAGTAATTTCCGGTGTCATTGGAGATATGAACTCGT
TAATGGTTTTCGGAAGAAAATATGTGGACCGGGATCTGGATGAAGAGTTA
GGTTTCAAGGCAGTCATAGATGAAATGTTGCACGTTGGTGCCTTGCCTAA
TCTAGGTGACTTTTTTCCATTCATGGCTGCACTAGACTTGCAAGGTTTGG
ATAGAAGGATGAAAGAATTGAGCAAGATTTTTGACGGGTTCCTAGAACGT
ATAATTGACGATCACTTGCTGAAAAAGACTGAGAATACCAAAAAGGGGGA
TTTCGTGGATACAATGCTGGCTGTTATGGAGGCCGGCGAAGCCGATTTTG
AATTCGATCGTAGACATGTTAAAGCTGTTCTGCTCGATATGCTTATCGCT
GGTATGGATACGTCTGCCAGCACAGTTGAATGGGCTCTGTCTGAGTTGAT
TAGACACCCTGAAATCACTAAGAAACTACAAAAAGAACTCGAGCAGGTTG
TTGGTATGGACCAGATGGTAGATGAGTCTCATCTAGACAAACTAGATTAC
CTGGATTCCGTCCTTAAAGAGACCTTAAGATTATACCCACCCGGTAGATT
GCTTGTCCATGAAACGATGGAAGAATGTACAGTTAACGGTTTTCATATTC
CAAAGGGTACATGGACTTTCGTCAATATGTGGAGTATAGGAAGAGATCCA
GCGATGTGGCATGAACCAGAAAAGTTTGTACCTGAACGCTTTGCCGGTGA
AAATTTAGATTTTCTAGGCCAAAATTTCAAATTCATTCCATTCGGTGCGG
GCCGAAGGTCATGCCCGGGTTTACAGTTGGGCTTAACTTTTGTTAGGTTG
GTGTTGGCACAACTTGTACATTGTTTTGACTGGGAACTTCCAAACGGAAT
GGTTCCGTCAGACTTGGATATGAACGAGAAATTCGGTATCGTCACTTCTA
GAGATAAGCATTTAATGGCTATTCCAACCTACAGATTAAATAAATAA

In at least one embodiment of the present disclosure, the CPR006 polypeptide of SEQ ID NO: 6 can be encoded for heterologous expression by the yeast codon-optimized nucleotide sequence of SEQ ID NO: 5.

(SEQ ID NO: 5)
ATGACCTCCGCACTCTACGCATCCGATTTGTTTAAGCAGTTGAAATCTAT
AATGGGAACCGACTCCTTGAGCGATGACGTGGTTTTAGTGATTGCGACTA
CTTCATTGGCCTTAGTAGCCGGATTCGTTGTACTTTTGTGGAAAAAAACA
ACTGCTGATCGCTCTGGTGAATTAAAACCACTAATGATCCCAAAGAGTCT
GATGGCTAAAGATGAAGATGATGATCTAGACCTAGGTTCAGGTAAAACAA
GAGTCTCTATATTTTTTGGCACCCAGACAGGTACGGCTGAAGGCTTTGCT
AAAGCATTGTCGGAAGAAATTAAGGCCAGATATGAAAAGGCAGCAGTCAA
GGTTATAGACCTGGACGATTACGCTGCGGACGACGACCAATACGAGGAAA
AACTAAAGAAGGAGACGTTGGCTTTTTTTTGTGTAGCTACATACGGCGAT
GGAGAACCGACAGACAATGCTGCTAGATTCTACAAGTGGTTCACTGAAGA
GAACGAACGTGACATAAAATTACAGCAACTTGCATACGGGGTGTTTGCCC
TTGGCAACCGACAATACGAACACTTTAATAAGATCGGGATCGTTCTAGAT
GAGGAGCTGTGCAAAAAAGGAGCAAAAAGGCTTATTGAGGTGGGTCTCGG
CGATGACGATCAAAGCATTGAGGATGATTTCAACGCGTGGAAAGAATCGT
TGTGGAGTGAATTGGATAAACTTTTAAAGGACGAAGATGATAAGAGTGTG
GCTACACCATATACTGCCGTTATTCCCGAGTATAGAGTCGTAACACACGA
TCCACGTTTCACAACTCAAAAGTCAATGGAATCAAATGTCGCCAATGGTA
ACACTACTATTGATATTCACCACCCTTGCAGAGTTGATGTAGCAGTACAA
AAAGAGCTTCATACCCATGAATCTGATAGGAGCTGTATACATTTGGAGTT
TGATATCAGCAGAACGGGGATAACTTATGAGACAGGAGATCATGTTGGTG
TCTATGCTGAGAATCATGTCGAGATTGTTGAAGAAGCTGGTAAACTACTG
GGGCACTCCTTAGATTTAGTTTTCTCGATTCATGCTGATAAAGAAGATGG
CTCACCTTTGGAAAGTGCTGTACCACCACCATTTCCTGGGCCTTGTACTT
TAGGAACCGGTCTGGCTAGGTATGCAGATTTATTGAATCCGCCACGAAAA
TCAGCACTAGTAGCTTTAGCTGCATATGCCACTGAACCTAGTGAAGCAGA
AAAATTAAAACATCTCACCAGTCCTGATGGTAAAGACGAATACTCACAGT
GGATTGTTGCATCACAAAGATCATTATTAGAAGTGATGGCAGCGTTTCCC
TCTGCCAAACCTCCACTTGGAGTCTTTTTCGCTGCCATTGCCCCGAGATT
GCAACCAAGATATTACAGTATTTCTTCTTCTCCAAGGTTGGCACCCAGCA
GAGTTCATGTCACTTCAGCGTTGGTTTATGGTCCAACCCCAACAGGTAGG
ATCCATAAAGGTGTTTGTTCCACTTGGATGAAAAATGCAGTCCCAGCTGA
AAAGTCTCACGAATGTTCTGGTGCCCCTATCTTTATAAGAGCTTCTAATT
TTAAGTTGCCCTCGAACCCGTCTACACCTATAGTTATGGTGGGTCCCGGA
ACCGGTCTAGCACCTTTCCGTGGTTTTTTGCAGGAAAGAATGGCGCTGAA
AGAAGACGGTGAAGAATTAGGCTCCAGTTTACTGTTCTTTGGCTGTCGGA
ACAGGCAAATGGATTTCATTTACGAAGACGAGCTTAACAATTTCGTCGAT
CAGGGCGTGATTTCCGAATTAATTATGGCCTTTTCGAGAGAGGGAGCACA
AAAGGAATATGTTCAGCATAAAATGATGGAGAAGGCTGCGCAAGTTTGGG
ACTTGATCAAGGAGGAAGGATATCTATATGTTTGCGGTGATGCCAAAGGT
ATGGCTAGAGATGTTCATCGTACATTACATACGATAGTGCAAGAACAAGA
AGGTGTATCTTCATCCGAAGCTGAAGCCATCGTTAAGAAGTTACAAACGG
AAGGTCGCTATCTCAGAGACGTATGGTAA

As a result of screening the 128 candidate homolog polypeptides for the desired CYP activity when expressed heterologously in two yeast screening systems, 15 exemplary polypeptide homologs were identified that exhibit the unexpected and surprising technical effect of THQ production when integrated in a recombinant host cell. These exemplary polypeptides and exemplary yeast optimized genes encoding them are summarized in Table 3 below (as well as in the following Examples and the accompanying Sequence Listing).

TABLE 3
Homolog CYP polypeptides with carvacrol/thymol to THQ conversion
activity

| | | | NT | AA |
|---|---|---|---|---|
| | | | SEQ | SEQ |
| | Source | | ID | ID |
| Name | Organism | Amino Acid Sequence | NO: | NO: |
| CYP016 | Salvia | MAWYWLALSFVILFLSFLQHLLRKTDLPPGPIALPL | 7 | 8 |
| | hispanica | IGHLHLLGKNLSRDLHRLAAKHGPIIFLRLGTIPAV | | |
| | | VVSSAAGAELVLKTHDLAFAGRTHHQAARYIGYDQR | | |
| | | NILFAPYGAYWRNMRRLCTVELLSSAKINEFRPIRR | | |
| | | AGMGEMVGSLRRDAERGEIVDLTARVSGVLGDMNCL | | |
| | | MVFGRKYVDRDLDEKLGFKAVIDETLQVAAIPNLGD | | |
| | | FFPFIAALDVQGLDRRMKKLSSIFDAFLDKIIDDHL | | |
| | | QRKLEKTHNHDFVDTMLAVMDSGEAGFEFDRRHVKA | | |
| | | VLLDILIGGMDTTLSTVEWAISELIRHPKVTKKLQN | | |
| | | ELERIVSLDQMVDESHLDKLEYLDLVIKETLRLHPP | | |
| | | GRLLVHESMENCTVNGFHIPKGTWTFVNIWTIGRDP | | |
| | | NFWHEPDEFVPERFVGENVDLLGQHFQLIPFGSGRR | | |
| | | SCPGQQLGLTLVKLVVAQLVHCFDWELPDGVEPRDL | | |
| | | DMTEHFGIVTSRDKHLMAIPSYRLYK | | |
| CYP017 | Salvia | MNFNILIDFLTSSLVAASIAWICILISRARKSSKLP | 9 | 10 |
| | splendens | PGPYGHPVIGNVLHLGPKPHRSLAKLSQKYGPVMSL | | |
| | | KLGSITTVVISSAETAKLVLQKHDSSFSNRTIPSAS | | |
| | | KALRHHDFSVVWLPVGSQWRKLRRICREQMFSALRL | | |
| | | DASQGLRREKLQKLRDFVAERCDRGAAVDVGDAAFT | | |
| | | TALNLMSASLFSAEFARFDSESSQEMKEVVWGVMKS | | |
| | | VGSPNFADYFPLLKPADPQGIFQAAEFNFGKLFAKL | | |
| | | DEIIDEKLKSGGEKRDLVEALLEINRRDEDQLSRED | | |
| | | IRHLLLDLFVAGTDTTSGTVEWAMTELIRNPQKLEK | | |
| | | LRNEIRSFMSEHGKQQVQESDISSLPYLQAVVKETF | | |
| | | RLHPTAPFLLPHKASSDVEINGYIVPENAQILINVW | | |
| | | ASGRDSNIWKNPNEFLPERFLNENGNVDFKGRDFEL | | |
| | | IPFGAGRRICPGLPLADRMVHLMLVTFVGNYEWKLE | | |
| | | NIKPEEMDMNENFGITLQKAVPLKAIPTKL | | |
| CYP018 | Salvia | MAWFWLALSFIILLSFLQHLLRRRDLPPGPIALPLI | 11 | 12 |
| | splendens | GHLHLLGKNLSRDLHRLAAKHGPIIFLRLGTIPAVV | | |
| | | VSSAAGAELVLKTHDLAFAGRTHHQAARYIGYDQRN | | |
| | | ILFAPYGAYWRNMRRLCTVELLSSAKINEFRPIRRA | | |
| | | GMGEMVGSLRRDAERGEIVDLTARVSGVLGDMNCLM | | |
| | | VFGRKYVDRDLDEKLGFKAVIDETLQVAAIPNLGDF | | |
| | | FPFIAALDLQGLDRRMKKLSSIFDAFLDKIIDDHLQ | | |
| | | RKPEKTHNRDFVDTMLAVMDSGEAGFEFDRRHVKAV | | |
| | | LLDILIGGMDTTLSTVEWAISELIRHPKVTKKLQSE | | |
| | | LERIVSLDEMVDESHLDKLEYLDLVVKETLRLHPPG | | |
| | | RLLVHESIENCTVNGFHIPKGTWTFVNIWTIGRDPN | | |
| | | FWHEPDEFVPERLVGKNVDLLGQHFQLIPFGSGRRS | | |
| | | CPGQQLGLTLVKLVVAQLVHCFDWELPDGVEPRDLD | | |
| | | MTEHFGIVTSRAKHLNSIPSYRLYK | | |
| CYP019 | Salvia | MDLLTSLIFVASIAWICILILTSNSRARKSSKLPPG | 13 | 14 |
| | splendens | PYGPPIIGNILHLGPKPHRSLAKLSQKYGPVMSLKL | | |
| | | GSITTVVISSAETAKLVLQKHDSSFSNRTIPSASKA | | |
| | | LRHHEFSVAWLPVGSQWRKLRKICREQMFSAPRLDS | | |
| | | SEGLRREKLHKLRDFVAERCDRGVAVNVGDAAFTTA | | |
| | | LNLMSASLFSVEFARFDSESSQEMKEVVWGVMKSVG | | |
| | | SPNLADYFPLLKPADPQRIFKAAEFNYGKLFAKLDE | | |
| | | IIDEKLKSGGDKRDLVEALLEINRRDEAQLSRDDIR | | |
| | | HLLLDLFVAGTDTTSGTVEWAMTELIRNPQKMAKLR | | |
| | | SEIRSFMSENGKQQVEESDITSLPYLQAVVKENFRL | | |
| | | HPAAPLLVPHKANCDVEINGYIVPENAQILINVWAS | | |
| | | GRDSNIWKNPNEFLPERFLNENGSVDFKGRDFELIP | | |
| | | FGAGRRICPGLPLADRMVHQMLVTFVGNYEWKLENT | | |
| | | KPEEMDMNENFGITLQKAVPLKAIPTKL | | |
| CYP020 | Salvia | MDFLTSSIFAASIAWICILILTSNSQARKLSKLPPG | 15 | 16 |
| | hispanica | PYGLPIIGNILQLGPKPHHSLAKLSQKYGPVMLLKL | | |
| | | GSITTVVISSAETAKLVLQKHDSSFSNRTIPSAITA | | |
| | | LRHHEFSVGWLPVGNQWRKLRKICKEQMFSAPRLDA | | |
| | | SEGLRREKLQKLRDFVAERCDRGTAVDVGEAAFTTS | | |
| | | LNLISASLFSMEFARFDSESSQEMKEVVWGVMKSIG | | |
| | | SPNLADYFPLLKPADPQGILKAAEFNFVKLFAKLDE | | |
| | | IIDEKLKSGVEKRDLVEALLEIHRRDEAQLSRDDIR | | |
| | | HLLLDLLLAGTDTTSGTMEWAMTELISNPQKMVKLR | | |
| | | DEIRSFMSENGKPQIDESDISSLPYLQAVVKETFRL | | |
| | | HPAAPFLLPHKASSYVEINGYIVPENAQILINVWAS | | |
| | | GRDSRIWRNPDEFLPERFLNENNNIDFKGRNFELIP | | |
| | | FSTGRRICPGLPLADRMVHQMLVTFVGNFEWKLENM | | |
| | | KLEEMDMNENFGITLQKAVPLKAIPTKL | | |
| CYP021 | Salvia | MNFNFLIDFLTSSLIAASIAWVCILISRARKSSKLP | 17 | 18 |
| | hispanica | PGPYGLPVIGNILHLGPKPHRSLAELSQKYGPVMSL | | |
| | | KLGSITTVVITSAETAKLVLQKHDSSFSNRTLPSAS | | |
| | | KALRHHEFSVAWLPVGSQWRKLRKICREQMFSAPRL | | |
| | | DASEGLRREKLQKLQDFVAERSDRGAAVNVGDAAFT | | |
| | | TALNLMSASLFSVEFARFDSESSQEMKEVVWGVMKA | | |
| | | VGSPNLADYFPLLKPADPQGIFKAAEYNYGKLFAKL | | |
| | | DEIIDEKLKSEGEKRDLVEALLEINRRDETQLSRDD | | |
| | | IRHLLLDLFVAGTDTTSGTVEWAMTELIRNPQKMAK | | |
| | | LRSEIRSFMSKNGKQQVEESDISSLPYLQAVVKENF | | |
| | | RLHPAAPFLVPHKANCDVEINDYIVPENAQILINVW | | |
| | | ASGRDSNIWKNPNEFLPERFLNENDNIDFKGRDFEL | | |
| | | IPFGAGRRICPGLPLADRMVHQMLVTFVGNYEWKLE | | |
| | | NMKPEEIDMNENFGITLQKAVPLKAIPTKL | | |
| CYP022 | Salvia | MDILTSSIILASIAWTCILISRARKLSKLPPGPYGL | 19 | 20 |
| | hispanica | PIIGNILHLGPTPHRSLAKLSHKYGPVMSLKLGSIT | | |
| | | TVVISSAETAKLVLQKYDSSFSNRTIPLASKALRHD | | |
| | | EFSVGWLPVGSQWRKLRKICREQMFSAPRLDASEGL | | |
| | | RREKLQKLRDFVANRCESGSAVDVGEAAFTTALNLI | | |
| | | SASLFSAEFARFDSESSQEMKEAVCGVMKSVGSPNF | | |
| | | ADYFPLLKPADPQGLLKAAELSTGMLFVKLDEIIDE | | |
| | | KLKSRGEKRDLVEALLEINQRDEAQLSRDDIRHLLL | | |
| | | DLLVAGTDTSSATVEWAMTELIRNPHKMTKLRNEIK | | |
| | | SFMSENGKQQVQESDIPLLPYLQAVVKETFRLHPAA | | |
| | | PFLLPHKASSDVEINGYIVPANAQILINVWASGRDS | | |
| | | RIWKHPDEFLPERFLNENEGVDFKGQDFELIPFSAG | | |
| | | RRICPGLPLADRMVHQMLVTFVENYEWKLENMKPEE | | |
| | | MDMNESFGITLRKAIPLKAIPTKL | | |
| CYP025 | Scutellaria | MICKEQMFSVSRLDASQSLRREKLQKLCDYVKECSE | 21 | 22 |
| | montana | TGRAVDIGEAAFTTSLNLMSATLFSKEFAQFNSDSS | | |
| | | QEMKDVVWGVMECVGSPNFADYFPVLKSIDPQGISK | | |
| | | QLEIHFEKLFAIFDGIIDDKMKSRAENNDLVEALID | | |
| | | INQRDEAELSRDEINHLLLDLFVAGVDTTSGTVEWA | | |
| | | MTELLRNPEKLSRVRNEIQDVIKQNGQVDESDIPQL | | |
| | | PYLQAVVKETFRLHPAAPFLVPHKANMDVEINGYIV | | |
| | | PKDTQVLVNVWASGRDPNIWKNPDSFEPERFLNDNV | | |
| | | HIDFKGKDFELIPFGSGRRICPGLPLANRMVHLMLA | | |
| | | TLVGKFDWKLEGILDMDEKFGLTLQKSIPLKAVPTK | | |
| | | L | | |
| CYP029 | Salvia | MDFLTSSVILASIAWICMLISRARKVSKLPPGPYGL | 23 | 24 |
| | hispanica | PIIGNILQLGPKPHHSLAKLSRKYGPVMSLKLGSIT | | |
| | | TVVISSAETAKLVLQKHDSSFSNRTVLSAITALRHH | | |
| | | EFSVGWLPVGNQWRKLRKICKEQMFSAPRLDASEGL | | |
| | | RREKLQKLQDYLAERCERGTAVDVGEAAFTTALNLM | | |
| | | SASLFSAEFVQFGSETSQEMKEAISSVVKSIGRPNL | | |
| | | ADYFPVLKAVDPQGIYKETELNVGRLFVKLDEIIDE | | |
| | | KLRSRGEKRDLVEVLLEIHRRDEAQLSRDDIRHLLL | | |
| | | DLLVAGTDTTSGTMEWAMTELIRNPQKMVKLRNEIR | | |
| | | SFMSESGKQQIEESDISSLSYLQAVVKENFRLHPAA | | |
| | | PFLVPHKANCDVEINGYIIPKNAQIFVNVWASGRDS | | |
| | | KIWKNPDEFLPERFLNENDNIDFKGRDFELIPFGSG | | |
| | | RRMCPGLPLADRMVHQMLVTFVGNYDWKLENMKPEE | | |
| | | MDMNENFGITLEKAIPLKAIPIKV | | |
| CYP030 | Buddleja | MFPIIGNILSLGPKPHQSLAKLSRKYGPVMSLKLGS | 25 | 26 |
| | sp. | LTTIVVSSPEAAKIVLQTHDLAFSSRSIPSAAGALN | | |
| | | HDKFSMVWLPVENQWRKLRKICKEQMFSVSRLDASQ | | |
| | | GLRREKLQKLCDYVNECCETGRAVDIGEAAFTTSLN | | |
| | | LISATLFSKEFAQFNTGSSQEMKEVVRGVMKCSGTP | | |
| | | NFADYFPVLKSVDPQGILKEAKIYFERLLAIFDEII | | |
| | | DEKLKNRSKSTEKNDLVDALIEINQRDEAELSRNDI | | |
| | | KHLLLDLFVAGSDTTSGTVEWAMTELLRNPEKMSRV | | |
| | | RGEIIDIIGANGQVQESDISNLPYLQAVVKETFRLH | | |
| | | PAGPFLVPHKAEANVEVNGYIVPKSAQILVNVWASG | | |
| | | RDSNVWSNADSFMPERFLDHQIDFRGKDFELIPFGA | | |
| | | GRRICPGLPLAYRMVHLMVATLVYNFKWKLEEGTKP | | |
| | | EEIDMNEKFGLSLQKTIPLKAIPTK | | |
| CYP056 | Erythranthe | MDFPTFLLVVLSIIWISTMILTFNSRARKSSKLPPG | 27 | 28 |
| | guttata | PNRFPIIGNILEIGPKPHKSLAKLAAKYGPVMSLKL | | |
| | | GTLTTVIISSPKAAKAVLQTHDLIFSSRTVPCAAEI | | |
| | | LSHHESSIAWLPAENRWRKLRKLCQEQMFSVARLDA | | |
| | | SQNLRREKLRKLRGYVGACSETGRAVDIGDAAFTTA | | |
| | | LNLMSATLFSLEFAPEFDSDSSQEMKDVVWGVMKCT | | |
| | | GSPNFADYFPILKSVDPQGILKEAKTYFQKLFVIFD | | |
| | | GIIDDKLKSGGESGKQDLLEALIEINRRDENELTKN | | |
| | | DIKHLLLDLFVAGTDTTSVTVEWAMAELLKHPHKMS | | |
| | | RVRQEIRDTVGENVEVEESDISRLPYLQAVVKETFR | | |
| | | LHPAAPFLVPHKVNADVEIKGFTVPKNAQVLVNVWA | | |
| | | SGRDPDTWPEAESFSPERFMDGRQIDIRGKDFELIP | | |
| | | FGSGRRICPGLPLAYRMVHLMLANLVGSFEWRLDGD | | |
| | | CKEIDMDDKFGLTLQKAIPLKAVPMKL | | |
| CYP088 | Erythranthe | MAWFWQTILIILLNWIIQKFLTLKKKQPPGPIGLPI | 29 | 30 |
| | guttata | IGHFHLLGKNPHQDLHRLAQKHGPIMYLRFGFVPTV | | |
| | | VFSSPAGAELVLKTHDLVFAGRASNQAAKHISYDGR | | |
| | | NIVFAQYGSYWRDMRKLATLNLLSTSKVSQFQPVRK | | |
| | | AELRLAVDSLRRAAAGRETVDVSEMIMGTIGDMVCL | | |
| | | MVFGRKFAGGELGGSGGGFKYVIDETFQVAAKPNLG | | |
| | | DFFPYMGTIDLQGLTRRMKELSTTFDGFLEKIIDDH | | |
| | | VLQNKQQKKENQDFVDTMMAIMDSGEAGFEFDRRHV | | |
| | | KAVLLDMLLGGTDTSSATIDWALAELIRHPQIMKKL | | |
| | | QQELEQVVGLDQYVEESHLEKLDYLNFVVKETFRLH | | |
| | | PVVPLFIHEALEDCTVEEFHIAKGTRIMVNVWSMSK | | |
| | | DPNVWQDPEKFFPERFIGSNLDLHGHDFELLPFGSG | | |
| | | RRSCPGLQLGLTTVRLVLAQLVHCFDWELPNGMSPS | | |
| | | ELDMSEHFGLVTGKDKHIMAIPVCRLHK | | |
| CYP091 | Verbascum | MAWIWISLCTIIVIYLLQELVNLNKKNKLPPGPIAL | 31 | 32 |
| | sp. | PILGHIHLLGKNPHQNLHHLAQKHGPIMFMRFGFKP | | |
| | | VIVISSPGAAELVLRTHDLVFACRPHSQASKHLGYN | | |
| | | RKNIIFAPYGPYWRNMRKLCTLQLLSQLKINQFQPM | | |
| | | RRTELGFLVSSLKQAAEIGETVDISAQISTLNRDMT | | |
| | | CLMVFGRKFGDGDLDEKGFKALADEIVQVAAVPNLG | | |
| | | DYFPLMGFLDLQGLTRRMKELSKTFDRFLDKIIDDH | | |
| | | IQCNNQEKENQDFVDTLMNIMMSGEAGGVLEFDRVH | | |
| | | VKAVLLDMLIGGMDTSAAAVEWTLSELIRHPKAMKK | | |
| | | LQKELKDVVGMDQTVDESHLDKLEYLDFVLKETLRL | | |
| | | HPVGPLLIHESIEDCKVDEFHIPKGSQLIVNAWSIG | | |
| | | RDPNVWKNPEKFIPERFIGSDIDLKGQHFELLPFGS | | |
| | | GRRGCPGMQLGLIVVRLMVAQLVHCFDWELHNGMLA | | |
| | | SELDMSEHFGIVTGRDTHLMVIPTYRL | | |
| CYP095 | Sanchezia | MAWIWVSISLIIFLWILHNQFTSNRKKKFPPGPIGL | 33 | 34 |
| | sp. | PVLGHLHLLGKNPHHDLHRLAQKHGPIMSLRFGLVP | | |
| | | TVVVSSPAGAELILKTHDLVFAGRPRYQAAKHLSYG | | |
| | | GRNILFAPYGNYWRNMRKLCTLELLSSSRINQFRPI | | |
| | | RTVELGFLIKALQSAAERGETVDVSARVSALNGDVI | | |
| | | SLMVFGRKFDDNELDERGFKAVIEETLAMAARPNLG | | |
| | | DFFPLIAAVDLQGLDRRMKELSGMFDGFLERIIEDH | | |
| | | RGKGKRKKEEQEDFVDTMVGILESGEAEFEFDIRHV | | |
| | | KAVLMDMLIGGMDTSTTAVEWVLSELIRHPKAMKKL | | |
| | | QQELEQVVGIDQLVDESHLDKLEYLNMTIKETLRLH | | |
| | | PVGPLLIHESMEDCTVSGFHIRKGSRIIVNVSSIGK | | |
| | | DPNVWQDPEKFMPERFVGSHVDLRG | | |
| CYP098 | Anticharis | MNWLWTILSAIVFLNLFQRLLSLKKKKRLPPGPRGL | 35 | 36 |
| | glandulosa | PVLGHFHLLGKNPHQDLCRLARKHGPIMYLRFGLVP | | |
| | | TIVVSSPAAAELFLKTYDLVFASRPPHQASKYISYD | | |
| | | QRNLTFGPYGPYWRDMRKLCTLELLSSLRISQFQAM | | |
| | | RKAELELLVNSLKQAALKEETVDLSSKVSSLSADMT | | |
| | | CLMVFGRKYVDRDLDEKGFKAVIMDALEVAAAFNLG | | |
| | | DYFPYLGVLDLQGLTRRMKALHKIFDGFLEKIIDDH | | |
| | | VRDKQDKKQSRDFVDVMMGIMESGEAGFEFDRRHIK | | |
| | | AVLLDMLVAGMDTSATTVEWTLSELLKHPTIMRKLQ | | |
| | | KELEDVVGMDHMVEESHLDQLEYLDLVIKEALRLHP | | |
| | | VAPLLLPHWSMEDCVVDGFHIPKNSRILVNVWAIGR | | |
| | | DPNVWPDPEAFSPERFVGCNIDLRGRDFQLIPFGSG | | |
| | | RRGCPGLQLGLTVVRLMVAQLVHCFDWKLPSGMKPN | | |
| | | ELDMSEHFGLVTSRANHVMAIPSYRL | | |

In at least one embodiment, when a host cell (e.g., S. cerevisiae or P. pastoris) is transformed with a heterologous nucleic acid encoding an exemplary homolog CYP polypeptide of the present disclosure (e.g., a CYP polypeptide of Table 3), and the recombinant host cell is fed the substrate(s) carvacrol and/or thymol, the product molecule, THQ is produced by the host cell. Furthermore, the THQ production is in greater yield relative to a comparable recombinant host cell integrated with a gene encoding a recombinant CYP polypeptide from Origanum vulgare of SEQ ID NO: 2 or 4. Without intending to be bound by any particular theory or mechanism, the enhanced yield of the THQ biosynthetic product is correlated with the one or more amino acid residue differences in the homolog CYP polypeptides of the present disclosure, as compared to the amino acid sequences of the CYP polypeptides of SEQ ID NO: 2 or 4, both of which have sequence identity of 76% or less to any of the 15 exemplary homolog polypeptides of Table 3.

Based on the correlation of recombinant polypeptide functional information provided herein with the sequence information provided in Table 3, the accompanying Sequence Listing, and/or the Examples disclosed herein, one of ordinary skill can recognize that the present disclosure of homolog CYP polypeptides provides a range of recombinant polypeptides having CYP activity capable of converting carvacrol and/or thymol to THQ, wherein the polypeptide comprises an amino acid sequence comprising one or more of the amino acid differences or sets of amino acid differences (relative to SEQ ID NO: 2 or 4) disclosed in any one of polypeptide homolog sequences of SEQ ID NO: 8, 10, 12, 14, 16, 18, 20, 22, 24, 26, 28, 30, 32, 34, and 36, and otherwise have at least 80%, at least 85% at least 90%, at least 91%, at least 92%, at least 93%, at least 94%, at least 95%, at least 96%, at least 97%, at least 98%, at least 99%, or at least 99.5% sequence identity to a sequence selected from the group consisting of SEQ ID NO: 8, 10, 12, 14, 16, 18, 20, 22, 24, 26, 28, 30, 32, 34, and 36.

Additionally, in at least one embodiment, a recombinant polypeptide of the present disclosure having CYP activity capable of converting carvacrol and/or thymol to THQ can have an amino acid sequence comprising one or more of the amino acid differences or sets of amino acid differences (relative to SEQ ID NO: 2 or 4) disclosed in any one of SEQ ID NO: 8, 10, 12, 14, 16, 18, 20, 22, 24, 26, 28, 30, 32, 34, and 36, and additionally have 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions.

In addition to the residue positions specified above, any of the homolog CYP polypeptides disclosed herein can further comprise other residue differences relative to the reference polypeptides of SEQ ID NO: 2 or 4 at other residue positions.

Residue differences at these other residue positions can provide for additional variations in the amino acid sequence without adversely affecting the ability of the recombinant polypeptide to carry out the desired biocatalytic conversion of carvacrol (compound 2a) and/or thymol (compound 2b) to THQ (compound 1a). In some embodiments, the recombinant polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40 residue differences at other amino acid residue positions as compared to SEQ ID NO: 2. In some embodiments, the number of differences can be 1, 2, 3, 4, 5, 6, 7, 8, 9, 10, 11, 12, 14, 15, 16, 18, 20, 22, 24, 26, 30, 35, and 40 residue differences at other residue positions. The residue difference at these other positions can include conservative changes or non-conservative changes. In some embodiments, the residue differences can comprise conservative substitutions and non-conservative substitutions as compared to the reference polypeptides of SEQ ID NO: 2 or 4.

In some embodiments, the recombinant homolog CYP polypeptides of the present disclosure can be in the form of fusion polypeptides in which the polypeptides are fused to other polypeptides, such as, by way of example and not limitation, antibody tags (e.g., myc epitope), purification sequences (e.g., His tags for binding to metals), and cell localization signals (e.g., secretion signals). Thus, the recombinant polypeptides described herein can be used with or without fusions to other polypeptides. It is also contemplated that the recombinant polypeptides described herein are not restricted to the genetically encoded amino acids. In addition to the genetically encoded amino acids, the polypeptides described herein may be comprised, either in whole or in part, of naturally occurring and/or synthetic non-encoded amino acids.

In another aspect, the present disclosure provides polynucleotides encoding the recombinant polypeptides having CYP activity capable of converting carvacrol and/or thymol to THQ with increased activity and/or yield as described herein (e.g., CYP polypeptides of Table 3). In at least one embodiment, the polynucleotide encoding a recombinant CYP polypeptide comprises an polynucleotide sequence that is at least about 70%, 75%, 80%, 85%, 90%, 91%, 92%, 93%, 94%, 95%, 96%, 97%, 98%, 99%, or more identical to a polynucleotide sequence encoding an exemplary polypeptide of SEQ ID NO: 8, 10, 12, 14, 16, 18, 20, 22, 24, 26, 28, 30, 32, 34, or 36. In some embodiments, the polynucleotide encodes a recombinant polypeptide comprising an amino acid sequence that has the percent identity described above and has one or more amino acid residue differences as compared to the polypeptide of SEQ ID NO: 8, 10, 12, 14, 16, 18, 20, 22, 24, 26, 28, 30, 32, 34, and 36.

In at least one embodiment, the polynucleotide has a sequence encoding a recombinant polypeptide of the present disclosure in which the encoding polynucleotide sequence has one or more neutral codon differences relative to a polynucleotide sequence of SEQ ID NO: 7, 9, 11, 13, 15, 17, 19, 21, 23, 25, 27, 29, 31, 33, and 35, which codon differences do not encode an amino acid difference but result in increased yield of the desired product compound (e.g., THQ) produced by a recombinant host cell in which the polynucleotide sequence is integrated.

It is also contemplated that the polynucleotides encoding the recombinant polypeptides having CYP activity and increased activity and/or yield as described herein, can include a combination of one or more codon differences relative to SEQ ID NO: 7, 9, 11, 13, 15, 17, 19, 21, 23, 25, 27, 29, 31, 33, and 35, wherein at least one of the codon differences encodes an amino acid difference as compared to the parent sequence and at least one codon difference is a neutral codon difference that does not encode an amino acid difference as compared to the parent sequence of SEQ ID NO: 8, 10, 12, 14, 16, 18, 20, 22, 24, 26, 28, 30, 32, 34, and 36 Accordingly, in at least one embodiment, the present disclosure provides a polynucleotide sequence encoding a recombinant polypeptide having CYP activity capable of converting carvacrol and/or thymol to THQ, wherein the polynucleotide sequence comprises a combination of a codon differences encoding an amino acid difference and a neutral codon difference.

In at least one embodiment, the polynucleotide comprises a sequence encoding an exemplary recombinant polypeptide having CYP activity as disclosed in Table 3 and the accompanying Sequence Listing. In at least one embodiment, the polynucleotide comprises a sequence of at least 80%, at least 85%, at least 90%, at least 95%, at least 97%, at least 98%, or at least 99% identity to a sequence selected from the group consisting of SEQ ID NO: 7, 9, 11, 13, 15, 17, 19, 21, 23, 25, 27, 29, 31, 33, and 35. In at least one embodiment, the polynucleotide comprises a codon degenerate sequence of a sequence selected from the group consisting of SEQ ID NO: 7, 9, 11, 13, 15, 17, 19, 21, 23, 25, 27, 29, 31, 33, and 35.

The polynucleotide sequences encoding the recombinant polypeptides of the present disclosure may be operatively linked to one or more heterologous regulatory sequences that control gene expression to create a recombinant polynucleotide capable of expressing the polypeptide. Expression constructs containing a heterologous polynucleotide encoding the recombinant polypeptide can be introduced into appropriate host cells to express the corresponding polypeptide. Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved transaminase enzymes disclosed herein. Thus, having identified a particular amino acid sequence, those skilled in the art could make any number of different nucleic acids by simply modifying the sequence of one or more codons in a way which does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein, including the amino acid sequences presented in Table 3 and the accompanying Sequence Listing.

The codons can be selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. It is contemplated that all codons need not be replaced to optimize the codon usage of the recombinant polypeptide since the natural sequence will comprise preferred codons and because use of preferred codons may not be required for all amino acid residues. Consequently, codon optimized polynucleotides encoding the recombinant polypeptide may contain preferred codons at about 40%, 50%, 60%, 70%, 80%, or greater than 90% of codon positions of the full-length coding region.

The present disclosure also provides an expression vector comprising a polynucleotide encoding a recombinant polypeptide having CYP activity capable of converting carvacrol and/or thymol to THQ, and one or more expression regulating regions such as a promoter, a terminator, a replication origin, or the like, depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the recombinant polypeptide at such sites. Alternatively, a polynucleotide sequence of the present disclosure may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression. The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one which, when introduced into the host cell, is integrated into the genome, and replicated together with the chromosome(s) into which it has been integrated. Furthermore, a single vector or plasmid or two or more vectors or plasmids which together contain the total DNA to be introduced into the genome of the host cell, or a transposon may be used. In at least one embodiment, the expression vector further comprises one or more selectable markers, which permit easy selection of transformed cells.

### Use in Recombinant Host Cells

The recombinant genes of the present disclosure that encode recombinant polypeptides with CYP activity capable of converting carvacrol and/or thymol to THQ (e.g., exemplary polypeptides of Table 3) can be incorporated in recombinant host cells to enable in vivo biosynthesis of the compound THQ and THQ derivative compounds. These recombinant host cells comprise a polynucleotide or expression vector that encodes the recombinant homolog polypeptide with CYP activity, wherein the polynucleotide is operatively linked to one or more control sequences for expression of the polypeptide in the host cell. Host cells for use in expressing recombinant genes encoding the polypeptides with CYP activity of the present disclosure are well known in the art and include but are not limited to, bacterial cells, such as E. coli, or fungal cells, such as Saccharomyces cerevisiae or Pichia pastoris, insect cells, such as Drosophila S2 and Spodoptera Sf9, animal cells, such as CHO, COS, BHK, 293, and plant cells. Appropriate mediums and growth conditions for culturing the recombinant host cells so that they express the polypeptide with CYP activity are well known in the art.

The recombinant host cells can comprise heterologous nucleic acids encoding not only polypeptides with CYP activity capable of converting carvacrol and/or thymol to THQ, and also other enzymes, such as CPR, and enzymes capable of producing other compounds, such as precursors for the substrates, carvacrol and/or thymol. As described elsewhere herein, nucleic acid sequences encoding pathway enzymes for producing such precursor compounds are known in the art and can readily be used in accordance with the present disclosure. Typically, the nucleic acid sequence encoding the enzymes which form a part of the pathway, further include one or more additional nucleic acid sequences, for example, a nucleic acid sequence controlling expression of the enzymes which form a part of the biosynthetic pathway, and these one or more additional nucleic acid sequences together with the nucleic acid sequence encoding the recombinant polypeptides with CYP activity can be considered a heterologous nucleic acid sequence. A variety of techniques and methodologies are available and well known in the art for introducing heterologous nucleic acid sequences, such as nucleic acid sequences encoding the enzymes (e.g., CYP and CPR), into a host cell so as to attain expression the host cell. Such techniques are well known to the skilled artisan and can be found in, for example, Sambrook et al., Molecular Cloning, a Laboratory Manual, Cold Spring Harbor Laboratory Press, 2012, Fourth Ed.

For example, the introduction of the heterologous nucleic acids can include integration of the nucleic acids into specific loci in the genome of a host cell via CRISPR-Cas9 and other techniques, some of which are demonstrated in the Examples herein. Such techniques are well known to the skilled artisan and can, for example, be found in Sambrook and other well-known sources. The number of copies of heterologous genes and their locus of integration in a recombinant host cell's genome can result in improved biosynthetic production of a desired product, such as THQ. For example, a heterologous nucleic acid encoding a polypeptide with CYP activity capable of converting carvacrol and/or thymol to THQ can be integrated into a host cell's genome in 1, 2, 3, 4, or more copies. Accordingly, it is contemplated that in the recombinant host cells of the present disclosure, the heterologous nucleic acid encoding the recombinant polypeptide having CYP activity can be integrated in the host cell's genome at one or more loci, including but not limited to the well-known genomic loci in Saccharomyces cerevisiae of X-2, X-4, XI-2, XII-4, NDE1, XII-5, Gal80, and ROQ1, or the loci in Pichia pastoris of AOX1, Int6, Int15, and HIS4.

One of ordinary skill will recognize that the heterologous nucleic acids encoding the recombinant enzymes with CYP activity capable of converting carvacrol and/or thymol to THQ, CPR activity, and any other pathway enzymes will further comprise transcriptional promoters capable of controlling expression of the enzymes in the recombinant host cell. Generally, the transcriptional promoters are selected to be compatible with the host cell, so that promoters obtained from bacterial cells are used when a bacterial host cell is selected in accordance herewith, while a fungal promoter is used when a fungal host cell is selected, a plant promoter is used when a plant cell is selected, and so on. Promoters useful in the recombinant host cells of the present disclosure may be constitutive or inducible, provided such promoters are operable in the host cells. Promoters that may be used to control expression in fungal host cells, such as Saccharomyces cerevisiae and Pichia pastoris, are well known in the art and include, but are not limited to, inducible promoters, such as a Gal1 promoter or Gal10 promoter, a constitutive promoter, such as an alcohol dehydrogenase (ADH) promoter, a glyceraldehyde-3-phosphate dehydrogenase (GPD) promoter, or an S. pombe Nmt, or ADH promoter. Exemplary promoters that may be used to control expression in bacterial cells can include the Escherichia coli promoters, lac, tac, trc, trp or the 77 promoter. Exemplary promoters that may be used to control expression in plant cells include, for example, a Cauliflower Mosaic Virus 35S promoter (Odell et al. (1985) Nature 313:810-812), a ubiquitin promoter (U.S. Pat. No. 5,510,474; Christensen et al. (1989)), or a rice actin promoter (McElroy et al. (1990) Plant Cell 2:163-171). Exemplary promoters that can be used in mammalian cells include, a viral promoter such as an SV40 promoter or a metallothionine promoter. All of these host cell promoters are well known by and readily available to one of ordinary skill in the art. Further nucleic acid control elements useful for controlling expression in a recombinant host cell can include transcriptional terminators, enhancers, and the like, all of which may be used with the heterologous nucleic acids incorporate in the recombinant host cells of the present disclosure.

A wide variety of techniques are well known in the art for linking transcriptional promoters and other control elements to heterologous nucleic acid sequences encoding pathway genes for biosynthesis of THQ. Such techniques are described in e.g., Sambrook et al., Molecular Cloning, a Laboratory Manual, Cold Spring Harbor Laboratory Press, 2012, Fourth Ed. Accordingly, in at least one embodiment, the heterologous nucleic acid sequences of the present disclosure comprise a promoter capable of controlling expression in a host cell, wherein the promoter is linked to a nucleic acid sequence encoding a recombinant polypeptide of the present disclosure having CYP activity capable of converting carvacrol and/or thymol to THQ, and as necessary, other enzymes constituting a pathway for production of a precursor, such as carvacrol and/or thymol, THQ, and/or THQ derivative. This heterologous nucleic acid sequence can be integrated into a recombinant expression vector which ensures good expression in the desired host cell, wherein the expression vector is suitable for expression in a host cell, meaning that the recombinant expression vector comprises the heterologous nucleic acid sequence linked to any genetic elements required to achieve expression in the host cell. Genetic elements that may be included in the expression vector in this regard include a transcriptional termination region, one or more nucleic acid sequences encoding marker genes, one or more origins of replication, and the like. In some embodiments, the expression vector further comprises genetic elements required for the integration of the vector or a portion thereof in the host cell's genome.

It is also contemplated that in some embodiments an expression vector comprising a heterologous nucleic acid of the present disclosure may further contain a marker gene. Marker genes useful in accordance with the present disclosure include any genes that allow the distinction of transformed cells from non-transformed cells, including all selectable and screenable marker genes. A marker gene may be a resistance marker such as an antibiotic resistance marker against, for example, kanamycin or ampicillin. Screenable markers that may be employed to identify transformants through visual inspection include β-glucuronidase (GUS) (U.S. Pat. Nos. 5,268,463 and 5,599,670) and green fluorescent protein (GFP) (Niedz et al., 1995, Plant Cell Rep., 14:403).

In at least one embodiment, the present disclosure also provides of a method for producing THQ, wherein a heterologous nucleic acid encoding a recombinant polypeptide having CYP activity capable of converting carvacrol and/or thymol to THQ (e.g., an exemplary engineered polypeptide of Table 3) can be introduced into a recombinant host cell. The recombinant host cell can then be used for production of the polypeptide or incorporated in a biocatalytic process that utilized the CYP activity of the recombinant polypeptide expressed by the host cell for the catalytic conversion of a substrate, e.g., the conversion of carvacrol and/or thymol. In at one embodiment, the recombinant host cell can further comprise a pathway of enzymes capable of producing a compound precursor (e.g., carvacrol) which can act as a substrate for the recombinant polypeptides with CYP activity and CPR activity. It is contemplated that a recombinant host cell comprising a heterologous nucleic acid encoding a recombinant polypeptide having CYP activity of the present disclosure can provide improved biosynthesis of a desired product compound (e.g., THQ or THQ derivative) in terms of titer, yield, and production rate, due to the improved characteristics of the expressed CYP activity in the cell associated with the amino acid and codon differences engineered in the gene.

Accordingly, in at least one embodiment, the present disclosure provides a method for producing THQ comprising: (a) culturing a recombinant host cell of the present disclosure in a suitable medium comprising carvacrol and/or thymol; and (b) recovering the produced THQ.

In at least one embodiment, it is contemplated the recombinant polypeptides with CYP activity of the present disclosure can be incorporated in any biosynthesis method requiring a CYP catalyzed biocatalytic step, whether in vivo or in vitro. For example, in at least one embodiment, the recombinant polypeptides having CYP activity (e.g., exemplary polypeptides of Table 3) can be used in a method for preparing compound (1a) or a derivative of compound (1a)

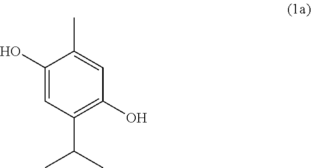

comprising contacting under suitable reactions conditions a recombinant polypeptide with CYP activity, wherein the polypeptide comprises an amino acid sequence of at least 80% sequence identity to a sequence selected from the group consisting of SEQ ID NO: 8, 10, 12, 14, 16, 18, 20, 22, 24, 26, 28, 30, 32, 34, and 36, and a substrate compound (2a) and/or a substrate compound (2b)

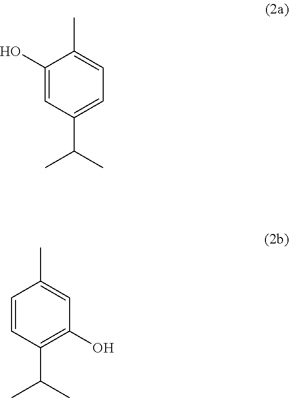

or a derivative of compound (2a) and/or a derivative of compound (2b).

The method comprises contacting a recombinant polypeptide having CYP activity of the present disclosure (e.g., an exemplary polypeptide of Table 3) under suitable reactions conditions with conditions compound (2a) and/or compound (2b), or a derivative of compound (2a) and/or a derivative of compound (2b). Exemplary conversions of THQ precursor compounds, carvacrol, compound (2a) and/or thymol, compound (2b) to THQ, compound (1a), and related structural analogs or derivatives of compound (1a) that are catalyzed by the recombinant polypeptides having CYP activity of the present disclosure can include: (1) conversion of thymol to THQ; (2) the conversion of carvacrol to THQ; and (3) conversion of a mixture of carvacrol and thymol to THQ. Accordingly, in at least one embodiment of the biosynthesis method for conversion a THQ precursor compound to THQ or a THQ structural analog or THQ derivative compound, the THQ precursor compound is carvacrol and/or thymol and the resulting product compound THQ.

The present disclosure also contemplates that the methods for biocatalytic conversion of a THQ precursor compound to a THQ or a THQ structural analog or a THQ derivative compound using an recombinant polypeptide having CYP activity of the present disclosure can further comprise chemical or biocatalytic steps carried out on the product compound of the bioconversion, including steps of product compound work-up, extraction, isolation, purification, and/or crystallization, each of which can be carried out under a range of conditions. For example, the biocatalytic conversion can further comprise a further biocatalytic or chemical step to form a derivative of compound (1a), such as compound (1b), compound (1c), compound (1d), compound (1e), compound (1f), compound (1g), compound (1h), compound (1i), compound (1j), compound (1k), compound (1l), compound (1m), compound (1n), compound (1o), compound (1p), compound (1q), compound (1r), compound (1s), compound (1t), compound (1u), compound (1v), compound (1w), compound (1x), compound (1y), compound (1z), compound (1aa), compound (1ab), compound (1ac), compound (1ad), compound (1ae), compound (1af), compound (1ag), compound (1ah), and compound (1ai).

In at least one embodiment of the method, a derivative of compound (2a) and/or a derivative of compound (2b) is used to prepare a derivative of compound (1a), wherein the derivative of compound (2a) and/or a derivative of compound (2b), wherein the derivative is selected from compound (2c), compound (2d), compound (2e), compound (2f), compound (2g), compound (2h), compound (2i), compound (2j), compound (2k), compound (2l), compound (2m), compound (2n), compound (2o), compound (2p), and mixture thereof.

Suitable reaction conditions for the biosynthesis of compounds such as THQ and THQ derivatives are known in the art and can be used with the recombinant polypeptides having CYP activity of the present disclosure. Additionally, suitable reaction conditions for the exemplary polypeptides of the present disclosure can be determined using routine techniques known in the art for optimizing biocatalytic reactions. It is contemplated that various ranges of suitable reaction conditions with the recombinant polypeptides of the present disclosure, including but not limited to ranges of pH, temperature, buffer, solvent system, substrate loading, polypeptide loading, co-substrate or co-factor loading, atmosphere, and reaction time. Suitable reaction conditions can be readily determined and optimized for particular reactions by routine experimentation that includes, but is not limited to, contacting the recombinant polypeptide and substrate under experimental reaction conditions of concentration, pH, temperature, solvent conditions, and detecting the production of the desired compound of structural formula (I). In at least one embodiment, the suitable reaction conditions comprise a reaction solution of ~pH 7-8, a temperature of 25C to 37C; optionally, the reaction conditions comprise a reaction solution of ~pH 7 and a temperature of ~30C. In at least one embodiment, the reaction solution is allowed to incubate at a temperature of 25C to 37 C for a reaction time of at least 1, 6, 12, 24, or 48 hours, before the amount of reaction product is determined.

Associated with the above-described biocatalytic methods, the disclosure also provides enzymatic reaction mixture compositions useful for enzymatic synthesis of the compound (1a) or a derivative of compound (1a). For example, in at least one embodiment the disclosure provides a composition comprising: (a) a recombinant polypeptide with CYP activity comprising an amino acid sequence of at least 80% sequence identity to a sequence selected from the group consisting of SEQ ID NO: 8, 10, 12, 14, 16, 18, 20, 22, 24, 26, 28, 30, 32, 34, and 36; and (b) a compound (2a) and/or compound (2b), or a derivative of compound (2a) and/or a derivative of compound (2b). In at least one embodiment, the composition further comprises a recombinant polypeptide with CPR activity; optionally, a polypeptide comprising an amino acid sequence of at least 80% sequence identity to SEQ ID NO: 6.

It is also contemplated that the composition can comprise other compounds useful in further biocatalytic or chemical steps, such as reagents useful in the production of derivatives of compound (1a), such as compound (1b), compound (1c), compound (1d), compound (1e), compound (1f), compound (1g), compound (1h), compound (1i), compound (1j), compound (1k), compound (1l), compound (1m), compound (1n), compound (1o), compound (1p), compound (1q), compound (1r), compound (1s), compound (1t), compound (1u), compound (1v), compound (1w), compound (1x), compound (1y), compound (1z), compound (1aa), compound (1ab), compound (1ac), compound (1ad), compound (1ae), compound (1af), compound (1ag), compound (1ah), and compound (1ai). Accordingly, in at least one embodiment, the composition can comprise a derivative of compound (2a) and/or a derivative of compound (2b), for example a derivative selected from compound (2c), compound (2d), compound (2e), compound (2f), compound (2g), compound (2h), compound (2i), compound (2j), compound (2k), compound (2l), compound (2m), compound (2n), compound (2o), compound (2p), and mixture thereof.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Establishment of CYP Activity for Conversion of Carvacrol and/or Thymol to Thymohydroquinone (THQ)

This example illustrates establishment of baseline activity of two CYP polypeptides from Origanum vulgare, CYP76S40 (CYP002; SEQ ID NO: 2) and CYP736A300 (CYP003; SEQ ID NO: 4) which have previously been shown to convert carvacrol and/or thymol to THQ in heterologous yeast systems (see e.g., Krause, et al. “The Biosynthesis of Thymol, Carvacrol, and Thymohydroquinone in Lamiaceae Proceeds via Cytochrome P450s and a Short-Chain Dehydrogenase.” Proceedings of the National Academy of Sciences 118, no. 52, Dec. 28, 2021).

### Material and Methods

A. Construction of Control S. cerevisiae Strains with Integrated CYP Genes Previously Identified.

Yeast codon-optimized genes encoding the enzymes CYP76S40 (SEQ ID NO: 1) and CYP736A300 (SEQ ID NO: 3) with previously reported activity to convert carvacrol and/or thymol to THQ, were synthesized by TWIST. The synthesized genes (also referred to herein as CYP002 and CYP003, respectively), were further amplified to contain homology sequences to the S. cerevisiae pGAL1 promoter and tPGK1 terminator with the wbOligos5915 and wbOligos5917 primers to create donor cassettes and further integrated into the X-4 locus of two distinct screening strains as described below.

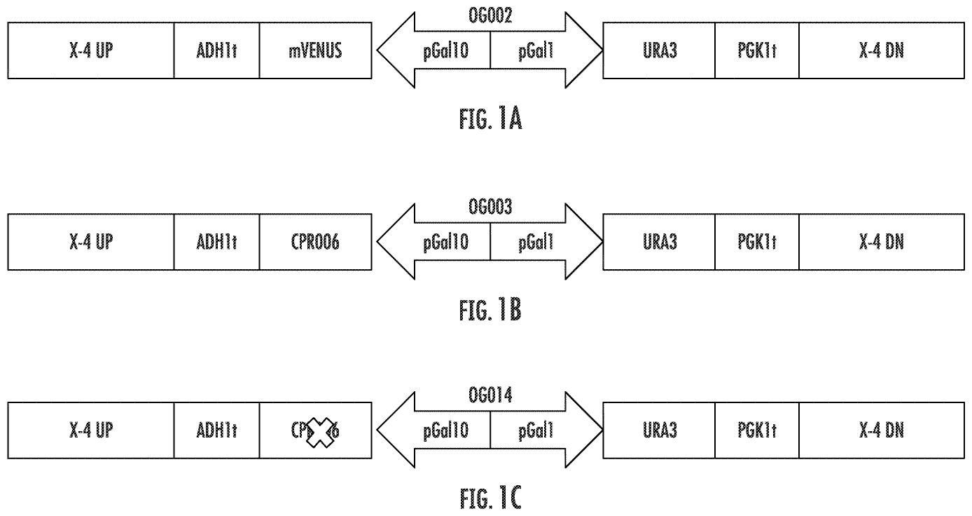

A first screening strain (OG002) was constructed expressing two landing sites at the X-4 locus, m-Venus and URA3. This OG002 screening strain did not contain a heterologous CPR reductase to function as the redox partner required for activity of CYP enzymes, instead relying on the endogenous yeast reductases (PGA3, AIM33, MCR1, CYB5, NCP1) to provide this CPR activity. FIG. 1A shows a schematic depiction of the X-4 insertion site of OG002 containing m-Venus and the URA3 gene under the bidirectional pGal10/1 promoter. This strain was not capable of converting carvacrol and/or thymol to THQ and therefore was also used as the negative control.

CYP002 and CYP003 integrated into OG002 generated strains OG008 and OG009 respectively. A second screening strain (OG003) was constructed to include a heterologous cytochrome P450 reductase (CPR006, SEQ ID NO: 6) to function as the redox partner required to work in tandem with the recombinant CYP enzyme. CPR006 (SEQ ID NO: 6) is the ATR1 CPR polypeptide from Arabidopsis thaliana and is the CPR sequence reported to be functioning along with CYP76S40 and CYP736A300, in a strain capable of converting carvacrol and/or thymol to THQ (Krause, et al, 2021). FIG. 1B shows a schematic depiction of the X-4 insertion site of OG003 containing CPR006 (SEQ ID NO: 6) and the URA3 gene (which was used as a landing site to integrate the two CYP genes) under the bidirectional pGal10/1 promoter. Using the Gal10/Gal1 promoters, these screening systems could be induced with galactose and/or via glucose depletion.

CYP002 (SEQ ID NO: 2) and CYP003 (SEQ ID NO: 4) integrated into OG003 generated strains OG004 and OG005 respectively. Additional copies of the resulting ADH1t_CPR006-pGAL 10-pGAL1_CYP002_PGK1t expression cassette were targeted to integration sites XI-2, XII-4, and X-2. Individual donors for integrating into each of these sites were generated by amplifying the CPR006-CYP002 cassette from OG004 then adding site-specific integration homology at both the 5′ and 3′ ends by overlap extension PCR. PCR fragments targeting the three different loci were amplified using the primers listed in Table 4 and the accompanying Sequence Listing.

TABLE 4

| PCR | Forward | SEQ ID | Reverse | SEQ ID |
|---|---|---|---|---|
| Fragment | primer | NO: | Primer | NO: |
| XI-2 up | wbOligos1104 | 37 | wbOligos 1238 | 43 |
| XI-2 dn | wbOligos1239 | 38 | wbOligos1107 | 44 |
| XII-4 up | wbOligos1272 | 39 | wbOligos5290 | 45 |
| XII-4 dn | wbOligos5291 | 40 | wbOligos1275 | 46 |
| X-2 up | wbOligos1202 | 41 | wbOligos5292 | 47 |
| X-2 dn | wbOligos5293 | 42 | wbOligos 1205 | 48 |

Primers used in overlap extension PCR are listed in Table 5 and the accompanying Sequence Listing.

TABLE 5

| | | SEQ | | SEQ |
|---|---|---|---|---|
| PCR | Forward | ID | Reverse | ID |
| Fragment | primer | NO: | Primer | NO: |
| CPR006-CYP002 | wbOligos1126 | 49 | wbOligos | 52 |
| at XI-2 | | | 1127 | |
| CPR006-CYP002 | wbOligos1276 | 50 | wbOligos | 53 |
| at XII-4 | | | 1277 | |
| CPR006-CYP002 | wbOligos1208 | 51 | wbOligos | 54 |
| at X-2 | | | 1209 | |

The resulting donors were transformed as a pool into OG004 which already contained a single copy of CPR006 and CYP002 at the X-4 locus as previously described. Individual clones were screened for THQ production using an HTP protocol (as described in Example 2C) and in shake flasks as described in section B below. A PCR confirmed two-copy strain, OGS034 (copies of CPR006/CYP002 verified at X-4 and X-2) and a three-copy strain OGS035 (copies of CPR006/CYP002 verified at X-4, XI-2, and XII-4) were identified with respective conversion shown in Table 7 (below).

### B. Screening of Initial Control Strains for THQ Bioconversion in Shake Flasks

To screen initial control strains, a shake flask protocol was developed to detect and quantify the bioconversion of carvacrol to THQ from the recombinant S. cerevisiae strains. Individual colonies of recombinant S. cerevisiae strains were picked into 500 mL flasks containing 2×YPD media (100 mL working volume). The flasks were incubated at 30° C. for 48 h with shaking at 250 rpm at 85% humidity. After this time, the strains were induced via the addition of galactose (1% final concentration) from a 40% stock solution. The flasks were incubated for a further 4 h before the biomass was harvested via centrifugation and the supernatant removed. A portion of the resulting biomass (2 g) was resuspended in 10 mL of bioconversion buffer (50 mg/L carvacrol, 0.1 M phosphate buffer, pH 7) in a 125 mL flask. Bioconversion was conducted at 30° C. for 24 h with shaking at 250 rpm at 85% humidity. After this time, a 300 μL aliquot was taken and extracted with MeOH (300 μL) with shaking at 250 rpm at 30° C. for a further 30 minutes. The biomass was then separated by centrifugation and the resulting supernatant diluted with MeOH (3× total dilution). The samples were then loaded onto an Agilent 1290 Infinity II UHPLC equipped with DAD and the compounds of interest were detected by monitoring at 274 nm and quantified relative to calibration curves which were prepared using serial dilutions of stock solutions of carvacrol and THQ.

UHPLC Instrumentation and parameters: UHPLC system: Agilent 1290 Infinity II UHPLC; Column: Agilent EclipsePlusC18 column 1.8 μm, 3.0×50 mm; Mobile phase A: 100% water+0.01% FA; Mobile phase B: 100% acetonitirile+0.01% FA; Gradient: 0-0.5 min isocratic 20% B, 0.5-2.5 min gradient to 60% B, 2.5-3.0 min isocratic 60% B; UV detection wavelength: 274 nm.

### Results

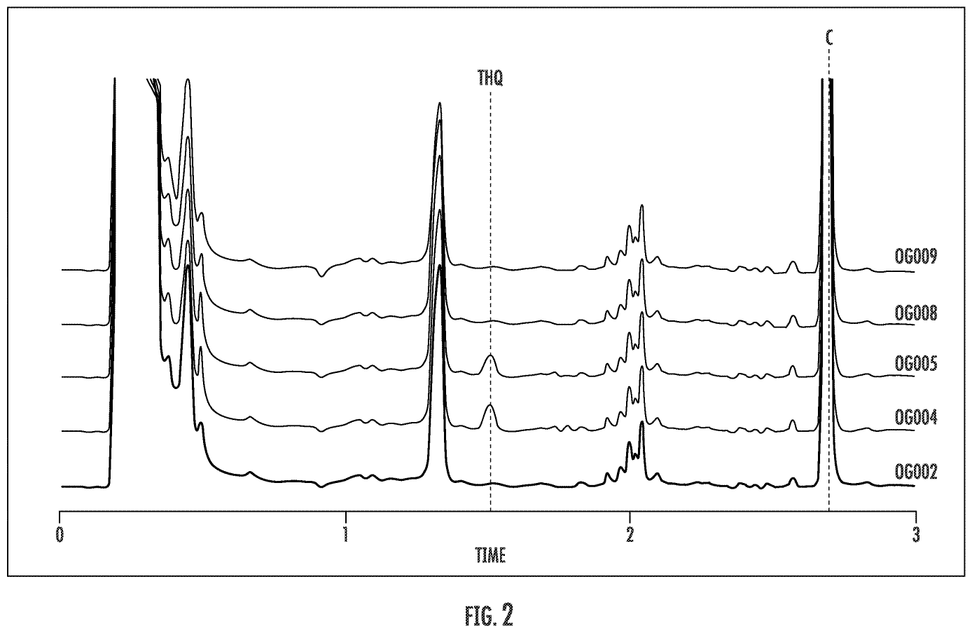

Exemplary UHPLC profiles for the strains OG004, OG005, OG008 and OG009 are shown in FIG. 2. The results of screening assays in shake flasks for the four S. cerevisiae strain builds (OG004, OG005, OG008, and OG009), along with the OG002 negative control are summarized in Table 6 below.

TABLE 6

| | | | Substrate | |
|---|---|---|---|---|
| Strain | CYP ID | CPR ID | Loading | Conversion |
| OG004 | CYP76S40/ | CPR006 | 50 mg/L | 9.3% |
| | CYP002 | | | |
| OG004 | CYP76S40/ | CPR006 | 100 mg/L | 3.7% |
| | CYP002 | | | |
| OG005 | CYP736A300/ | CPR006 | 50 mg/L | 10.5 % |
| | CYP003 | | | |
| OG005 | CYP736A300/ | CPR006 | 100 mg/L | 5.3% |
| | CYP003 | | | |
| OG008 | CYP76S40/ | Yeast | 50 mg/L | 0% |
| | CYP002 | endogenous | | |
| OG008 | CYP76S40/ | Yeast | 100 mg/L | 0% |
| | CYP002 | endogenous | | |
| OG009 | CYP736A300/ | Yeast | 50 mg/L | 0% |
| | CYP003 | endogenous | | |
| OG009 | CYP736A300/ | Yeast | 100 mg/L | 0% |
| | CYP003 | endogenous | | |

Results of HTP screening of the multiple copy strain builds in S. cerevisiae are summarized in Table 7 below.

TABLE 7

| | Copies of | Carvacrol | Carvacrol to THQ |
|---|---|---|---|
| Strain | CYP002 | Loading (mg/L) | % Conversion |
| OG004 | 1 | 50 | 3.5 |
| OGS034 | 2 | 50 | 12.7 |
| OGS035 | 3 | 50 | 29.3 |

### Example 2: Identification of Homologs of CYP002 and CYP003 with CYP Activity for Conversion of Carvacrol and/or Thymol to Thymohydroquinone (THQ)

This example illustrates a study to identify and screen candidate genes from various organisms that encode polypeptides with CYP activity capable of converting carvacrol and/or thymol to THQ when expressed heterologously in yeast.

### Material and Methods

### A. Search for Candidate CYP Homologs

The amino acid sequences of CYP002 (SEQ ID NO: 2) and CYP003 (SEQ ID NO: 4) were used to conduct sequence searches with a 60% sequence identity cut-off for genes encoding homologous polypeptide sequences. A total of 128 unique homologs from 45 different plant species were identified from public databases (NCBI, OneKP), including 28 homologs from 14 species that had >70% sequence homology. 96 of these unique homologs were selected to be synthesized and further evaluated for activity to convert carvacrol and/or thymol to THQ.

B. Construction of S. cerevisiae Strains with Integrated CYP Homolog Genes

A third screening strain (OG014) was constructed to include a non-functional cytochrome P450 reductase (CPR006 with 1 base pair deletion causing a frameshift) unable to act as the redox partner, required to work in tandem with a CYP enzyme. FIG. 1C shows a schematic depiction of the X-4 insertion site of OG014 containing a non-functional CPR006 and the URA3 gene (which was used as a landing site to integrate various CYP homolog genes) under the bidirectional pGal10/1 promoter.

The 96 candidate homolog genes were first amplified using primers wbOligos5945 (SEQ ID NO: 77) and wbOligos5377 (SEQ ID NO: 57). To facilitate integration, a fragment containing the pGAL10/1 promoter and a fragment containing the tPGK1 terminator and a portion of the S. cerevisiae X4 locus were added on to the amplified fragments via overlap-extension PCR using primers wbOligos4668 (SEQ ID NO: 56) and wbOligos1135 (SEQ ID NO: 55) to create donor cassettes. The resulting donor cassettes were transformed into two different screening strains, OG003 and OG014 to identify CYP enzymes that had activity with and active CPR006 or inactive CPR006 (endogenous reductases) respectively.

### C. HTP Screening of Strains for THQ Bioconversion

A HTP screening assay was developed to detect and quantify the bioconversion of carvacrol to THQ from the recombinant S. cerevisiae strains. Individual colonies of recombinant S. cerevisiae strains were picked into 96-well plates containing 2×YPD media (300 μL per well) using a QPix™ 420 colony picking system. The plates were incubated at 30° C. for 24 h with shaking at 250 rpm at 85% humidity. After this time, the strains were sub-cultured (40 μL inoculation volume per strain) into a second 96-well plate containing 2×YPD media (1.2 mL per well) using an Agilent Bravo automated liquid handling platform. The plates were incubated at 30° C. for 48 h with shaking at 250 rpm at 85% humidity. After this time, a solution of galactose (40% stock solution) was added using the Bravo (1% final concentration) and the plates were further incubated for 4 hours to induce protein production. After this time, the biomass was harvested via centrifugation and the supernatant removed. The resulting biomass was resuspended in 300 μL of bioconversion buffer (50 mg/L carvacrol, 0.1 M phosphate buffer, pH 7) using the Agilent Bravo automated liquid handling platform and the plates were incubated at 30° C. for 24 h with shaking at 250 rpm at 85% humidity. After this time, MeOH (300 μL) was added using the Bravo and the plates were incubated at 30° C. with shaking at 250 rpm at 85% humidity for a further 30 minutes. The biomass was then separated by centrifugation and the resulting supernatant diluted with MeOH (3×total dilution) using the Agilent Bravo automated liquid handling platform. The samples were then loaded onto an Agilent 1290 Infinity II UHPLC equipped with DAD and the compounds of interest were detected by monitoring at 274 nm and quantified relative to calibration curves which were prepared using serial dilutions of stock solutions of carvacrol and THQ.

UHPLC Instrumentation and parameters: Identical to those described in Example 1 (above).

### Results

The 96 CYP homologs were screened in S. cerevisiae (integrated to replace URA3 in OG003 and OG014) and compared to the control strain, OG004, which contains the gene encoding CYP002 (SEQ ID NO: 1) and CPR006 (SEQ ID NO: 5). 15 unique CYP homologs were identified with varying activity toward converting carvacrol and/or thymol to THQ. Of these 15 homologs the CYP identified from Salvia hispanica, CYP029 (SEQ ID NO: 24), was shown to fully convert 50 mg/L of carvacrol substrate to THQ.

Results obtained with screening strain 1 (OG003) with an integrated functional CPR006 gene are shown in Table 5 below.

TABLE 5

| | | | | Carvacrol |
|---|---|---|---|---|
| | | | Carvacrol | to THQ |
| | | AA | to THQ | % |
| | | SEQ | % | Conversion |
| | | ID | Conversion | FIOPC |
| CYP ID | Organism | NO: | (Mean) | (Mean) |
| CYP002 | Origanum vulgare | 2 | 4.6 | 1.0 |
| CYP016 | Salvia hispanica | 8 | 3.3 | 0.7 |
| CYP018 | Salvia splendens | 12 | 3.6 | 0.7 |
| CYP020 | Salvia hispanica | 16 | 11.1 | 2.6 |
| CYP021 | Salvia hispanica | 18 | 15.7 | 3.7 |
| CYP022 | Salvia hispanica | 20 | 61.3 | 14.3 |
| CYP029 | Salvia hispanica | 24 | 100.0 | 23.4 |
| CYP030 | Buddleja sp. | 26 | 1.6 | 0.4 |
| CYP088 | Erythranthe guttata | 30 | 14.1 | 3.3 |
| CYP091 | Verbascum sp. | 32 | 5.0 | 1.2 |
| CYP095 | Sanchezia sp. | 34 | 0.8 | 0.2 |
| CYP098 | Anticharis | 36 | 2.1 | 0.5 |
| | glandulosa | | | |

Results obtained with screening strain 3 (OG014) with an integrated non-functional CPR006 gene (endogenous reductases only) are shown in Table 6 below.

TABLE 6

| | | | Carvacrol | Carvacrol |
|---|---|---|---|---|
| | | AA | to THQ | to THQ |
| CYP | | SEQ ID | % Conversion | % Conversion |
| ID | Organism | NO: | (Mean) | FIOPC (Mean) |
| CYP002 | Origanum | 2 | 3.7 | 1.0 |
| | vulgare | | | |
| CYP017 | Salvia | 10 | 2.0 | 0.5 |
| | splendens | | | |
| CYP019 | Salvia | 14 | 2.1 | 0.6 |
| | splendens | | | |
| CYP020 | Salvia | 16 | 2.8 | 0.7 |
| | hispanica | | | |
| CYP021 | Salvia | 18 | 2.0 | 0.5 |
| | hispanica | | | |
| CYP025 | Scutellaria | 22 | 1.9 | 0.5 |
| | montana | | | |
| CYP029 | Salvia | 24 | 6.5 | 1.8 |
| (strain | hispanica | | | |
| OG027) | | | | |
| CYP056 | Erythranthe | 28 | 6.2 | 1.7 |
| (strain | guttata | | | |
| OG026) | | | | |

### Example 3: Construction of Pichia Strains with CYP Activity for Conversion of Carvacrol and/or Thymol to Thymohydroquinone (THQ)

This example illustrates a study to build strains of Pichia pastoris with heterologous genes encoding an enzyme with CYP activity capable of converting carvacrol and/or thymol to THQ.

### Material and Methods

A. Construction of Pichia Strains Expressing CYP Genes Capable of Converting Carvacrol and/or Thymol to THQ

To improve upon the bioconversion of carvacrol and/or thymol to THQ in S. cerevisiae, the genes encoding CYP002 (SEQ ID NO: 1) and CPR006 (SEQ ID NO: 6) were integrated into the P. pastoris genome under the bidirectional pCAT1:pFDH1 promoter system (SEQ ID NO: 83) and tDAS1 and tDAS2 terminators SEQ ID NOs: 84 and 85, respectively, to generate a single copy strain (OGP010) as follows. A single plasmid (wbplasmid162; SEQ ID NO: 79) containing a zeocin resistance marker, a pUC origin of replication, a HIS4 homologous sequence (SEQ ID NO: 86), and a cassette comprising of tDAS1:CPR006:pCAT1:pFDH1:CYP002:tDAS2 was assembled. Sequences were amplified using PCR using the following primers: wboligos5866 (SEQ ID NO: 58), wboligos5867 (SEQ ID NO: 59), wboligos5868 (SEQ ID NO: 60), wboligos5869 (SEQ ID NO: 61), wboligos5870 (SEQ ID NO: 62), wboligos5871 (SEQ ID NO: 63), wboligos5872 (SEQ ID NO: 64), and wboligos5873 (SEQ ID NO: 65).

This was followed by agarose-gel purification and assembly using NEB's HiFi DNA Assembly Master Mix (catalog no. E2621X) according to manufacturer's instructions. Three microliters of the HiFi assembly were used to transform E. coli competent cells and plated on low-salt LB media containing zeocin. Plasmids were extracted from cultures inoculated by colonies using GeneJet Plasmid Miniprep Kit (ThermoFisher catalog no. K0503) and sequence verified. The sequence-confirmed plasmid was digested with Bbvcl and cleaned using Zymo Research's DNA Clean and Concentrator Kit (catalog no. D4014). The cut site, found in the HIS4 homologous sequence, linearized the plasmid, allowing for homologous integration at the HIS4 locus in Pichia competent cells, which were prepared from the Δaox1 BG-11 strain using Thermo's Pichia EasyComp Transformation Kit (catalog no. K173001) according to manufacturer's instructions. Briefly, 5 μL of linearized plasmid (5 μg total) was used to transform a 50 μL aliquot of BG-11 competent cells and plated on YPDS media plates containing zeocin. After 3 days of growth, colonies were confirmed for integration of the linearized plasmid using PCR and genomic DNA from each colony.

A recombinant Pichia host cell strain (OGP011) with one copy of CYP003 (SEQ ID NO: 3) and CPR006 (SEQ ID NO: 5) was also created as follows. A single plasmid (wbplasmid163; SEQ ID NO: 80) containing a zeocin resistance marker, a pUC origin of replication, a HIS4 homologous sequence (SEQ ID NO: 86), and a cassette comprising of tDAS1:CPR006:pCAT1:pFDH1:CYP003:tDAS2 was assembled with the method described above. Sequences were amplified using PCR using the following primers: wboligos5866, wboligos5867, wboligos5868, wboligos5869, wboligos5870, wboligos5871, wboligos5872, and wboligos5873.

Following plasmid recovery and sequence confirmation, a single plasmid was digested with Bbvcl and cleaned using Zymo Research's DNA Clean and Concentrator Kit (catalog no. D4014). The cut site, found in the HIS4 homologous sequence, linearized the plasmid, allowing for homologous integration at the HIS4 locus in Pichia competent cells, which were prepared from the Δaox1 BG-11 strain using Thermo's Pichia EasyComp Transformation Kit (catalog no. K173001) according to manufacturer's instructions. Briefly, 5 microliters of linearized plasmid (5 μg total) was used to transform a 50-microliter aliquot of BG-11 competent cells and plated on YPDS media plates containing zeocin. After 3 days of growth, colonies were confirmed for integration of the linearized plasmid using PCR and genomic DNA from each colony.

A recombinant Pichia host cell strain (OGP024) with one copy of a gene encoding CYP056 (SEQ ID NO: 28) and a gene encoding CPR006 (SEQ ID NO: 6) was also created as follows. A single plasmid (wbplasmid192; SEQ ID NO: 82) containing a zeocin resistance marker, a pUC origin of replication, a HIS4 homologous sequence (SEQ ID NO: 86), and a cassette comprising of tDAS1:CPR006:pCAT1:pFDH1:CYP056:tDAS2 was assembled with the method described above. As wbplasmid192 (SEQ ID NO: 82) shares the same sequence as wbplasmid162 (SEQ ID NO: 79) except for the CYP002 CDS, inverse PCR was used to amplify the vector minus the CYP002 CDS using primers wboligos6113 (SEQ ID NO: 76) and wboligos6114 (SEQ ID NO: 77). CYP056 (SEQ ID NO: 28) was amplified from S. cerevisiae strain OG026 using primers wboligos6112 (SEQ ID NO: 75) and wboligos6115 (SEQ ID NO: 78). Following plasmid recovery and sequence confirmation, a single plasmid was digested with Bbvcl and cleaned using Zymo Research's DNA Clean and Concentrator Kit (catalog no. D4014). The cut site, found in the HIS4 homologous sequence, linearized the plasmid, allowing for homologous integration at the HIS4 locus in Pichia competent cells, which were prepared from the Δaox1 BG-11 strain using Thermo's Pichia EasyComp Transformation Kit (catalog no. K173001) according to manufacturer's instructions. Briefly, 5 microliters of linearized plasmid (5 μg total) was used to transform a 50 microliter aliquot of BG-11 competent cells and plated on YPDS media plates containing zeocin. After 3 days of growth, colonies were confirmed for integration of the linearized plasmid using PCR and genomic DNA from each colony.

A recombinant Pichia host cell strain (OGP025) with one copy of a gene encoding CYP029 (SEQ ID NO: 24) and a gene encoding CPR006 (SEQ ID NO: 6) was also created as follows. A single plasmid (wbplasmid191; SEQ ID NO: 81) containing a zeocin resistance marker, a pUC origin of replication, a HIS4 homologous sequence (SEQ ID NO: 86), and a cassette comprising of tDAS1:CPR006:pCAT1:pFDH1:CYP029:tDAS2 was assembled with the method described above. As wbplasmid191 shares the same sequence as wbplasmid162 except for the CYP002 CDS, inverse PCR was used to amplify the vector minus the CYP002 CDS using primers wboligos6103 and wboligos6100. The gene encoding CYP056 (SEQ ID NO: 28) was amplified from S. cerevisiae strain OG027 gDNA using primers wboligos6101 (SEQ ID NO: 72) and wboligos6102 (SEQ ID NO: 73). Following plasmid recovery and sequence confirmation, a single plasmid was digested with Bbvcl and cleaned using Zymo Research's DNA Clean and Concentrator Kit (catalog no. D4014). The cut site, found in the HIS4 homologous sequence, linearized the plasmid, allowing for homologous integration at the HIS4 locus in Pichia competent cells, which were prepared from the Δaox1 BG-11 strain using Thermo's Pichia EasyComp Transformation Kit (catalog no. K173001) according to manufacturer's instructions. Briefly, 5 μL of linearized plasmid (5 μg total) was used to transform a 50 μL aliquot of BG-11 competent cells and plated on YPDS media plates containing zeocin. After 3 days of growth, colonies were confirmed for integration of the linearized plasmid using PCR and genomic DNA from each colony.

### B. Analysis of Strains for THQ Bioconversion

Screening of the recombinant Pichia strains for bioconversion of carvacrol and/or thymol to THQ was carried out according to the following assay: individual colonies of recombinant P. pastoris strains were picked into 500 mL baffled conical flasks containing BMGY media (100 mL working volume). The flasks were incubated at 30° C. for 72 h with shaking at 250 rpm at 85% humidity and were supplemented with glycerol (2% final concentration) every 24 hours. After this time, the biomass was harvested via centrifugation and the supernatant was discarded. The biomass was then resuspended in BMMY and incubated at 30° C. with shaking at 250 rpm at 85% humidity for 6 hours to induce protein production. After this time, the biomass was harvested via centrifugation and the supernatant removed. A portion of the resulting biomass (2 g) was resuspended in 10 ml of bioconversion buffer (50-250 mg/L 3-KCA, 0.1 M phosphate buffer, 2% MeOH, 2 mM 5-ALA, pH 7) and the flask was incubated at 30° C. for 24 h with shaking at 250 rpm at 85% humidity. The extraction, sample preparation and analytical methods are identical to those described in above examples.

### Results

Results obtained from recombinant Pichia strains are shown below in Table 7.

TABLE 7

| | | | Carvacrol | Carvacrol |
|---|---|---|---|---|
| | | CYP AA | Loading | to THQ |
| Strain ID | CYP ID | SEQ ID NO: | (mg/L) | % Conversion |
| OGP010 | CYP002 | 2 | 50 | 58 |
| OGP011 | CYP003 | 4 | 50 | 22 |
| OGP024 | CYP056 | 28 | 100 | 97 |
| OGP025 | CYP029 | 24 | 100 | 100 |
| OGP025 | CYP029 | 24 | 250 | 95 |
| OGP025 | CYP029 | 24 | 500 | 31 |

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. A recombinant host cell comprising a heterologous nucleic acid encoding a polypeptide with CYP activity capable of converting carvacrol and/or thymol to THQ, wherein the polypeptide comprises an amino acid sequence of at least 80%, at least 85%, at least 90%, at least 91%, at least 92%, at least 93%, at least 94%, at least 95%, at least 96%, at least 97%, at least 98%, at least 99%, or at least 99.5% sequence identity to a sequence selected from the group consisting of SEQ ID NO: 24, 8, 10, 12, 14, 16, 18, 20, 22, 26, 28, 30, 32, 34, and 36.

2. The cell of claim 1, wherein the heterologous nucleic acid further encodes a polypeptide with CPR activity; optionally, wherein the polypeptide with CPR activity comprises an amino acid sequence of at least 90% sequence identity to SEQ ID NO: 6.

3. The cell of claim 1, wherein the heterologous nucleic acid is integrated into one or more sites in the host cell genome; optionally, integrated at three or more sites.

4. The cell of claim 1, wherein the source organism of the host cell is selected from Pichia pastoris, Saccharomyces cerevisiae, Yarrowia lipolytica, and Escherichia coli.

5. The cell of claim 1, wherein the recombinant host cell is Pichia pastoris and the heterologous nucleic acid is integrated at one or more sites in the genome selected from AOX1, Int6, Int15, and HIS4; optionally, integrated at three or more sites.

6. The cell of claim 1, wherein the recombinant host cell is Saccharomyces cerevisiae and the heterologous nucleic acid is integrated at one or more sites in the genome selected from X-2, X-4, XI-2, XII-4, NDE1, XII-5, Gal80, and ROQ1; optionally, integrated at three or more sites

7. The cell of claim 1, wherein the heterologous nucleic acid is under the control of a promoter system selected from pGal1/10, and pCAT1:pFDH1.

8. The cell of claim 1, wherein the heterologous nucleic acid comprises:
(a) a sequence of at least 80% identity to a sequence selected from the group consisting of SEQ ID NO: 23, 7, 9, 11, 13, 15, 17, 19, 21, 25, 27, 29, 31, 33, and 35; or
(b) a codon degenerate sequence of a sequence selected from the group consisting of SEQ ID NO: 23, 7, 9, 11, 13, 15, 17, 19, 21, 25, 27, 29, 31, 33, and 35.

9. A polynucleotide comprising a sequence of at least 80% identity to a sequence selected from the group consisting of SEQ ID NO: 23, 7, 9, 11, 13, 15, 17, 19, 21, 25, 27, 29, 31, 33, and 35.

10. An expression vector comprising the polynucleotide of claim 9.

11. The expression vector of claim 10, further comprising a polynucleotide sequence of at least 80% identity to SEQ ID NO: 5.

12. The expression vector of any one of claims 10-11 further comprising a control sequence; optionally, wherein the control sequence comprises a promoter system selected from pGal1/10, and pCAT1:pFDH1.

13. A host cell comprising the polynucleotide claim 9 or the expression vector of any one of claims 10-11.

14. The host cell of claim 13, wherein the source organism of the host cell is selected from Pichia pastoris, Saccharomyces cerevisiae, Yarrowia lipolytica, and Escherichia coli.

15. A method for producing THQ comprising:
(a) culturing a recombinant host cell of any one of claims 1-8, 13-14 in a suitable medium comprising carvacrol and/or thymol; and
(b) recovering the produced THQ.

16. A method for preparing compound (1a) or a derivative of compound (1a) 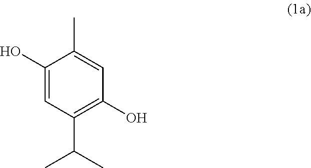
comprising contacting under suitable reactions conditions compound (2a) and/or compound (2b) or a derivative of compound (2a) and/or a derivative of compound (2b) 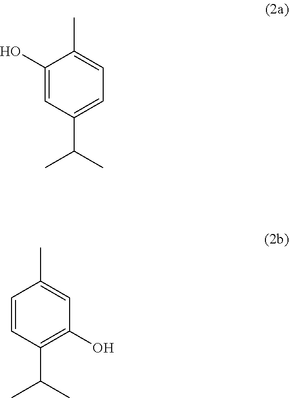
with a recombinant polypeptide with CYP activity, wherein the recombinant polypeptide with CYP activity comprises an amino acid sequence of at least 80% sequence identity to a sequence selected from the group consisting of SEQ ID NO: 24, 8, 10, 12, 14, 16, 18, 20, 22, 26, 28, 30, 32, 34, and 36.

17. The method of claim 16, wherein the method further comprises contacting with a recombinant polypeptide with CPR activity; optionally, wherein the recombinant polypeptide with CPR activity comprises an amino acid sequence of at least 80% sequence identity to SEQ ID NO: 6.

18. The method of any one of claims 16-18, wherein the contacting with the polypeptide with CYP activity occurs in the presence of host cell that expresses the polypeptide.

19. The method of any one of claims 16-18, wherein the contacting with the polypeptide with CYP activity occurs in a cell-free system.

20. The method any one of claims 16-19, wherein a derivative of compound (1a) is prepared, wherein the derivative is selected from compound (1b), compound (1c), compound (1d), compound (1e), compound (1f), compound (1g), compound (1h), compound (1i), compound (1j), compound (1k), compound (1l), compound (1m), compound (1n), compound (1o), compound (1p), compound (1q), compound (1r), compound (1s), compound (1t), compound (1u), compound (1v), compound (1w), compound (1x), compound (1y), compound (1z), compound (1aa), compound (1ab), compound (1ac), compound (1ad), compound (1ae), compound (1af), compound (1ag), compound (1ah), and compound (1ai).

21. The method any one of claims 16-20, wherein a derivative of compound (2a) and/or a derivative of compound (2b) is used to prepare a derivative of compound (1a), wherein the derivative is selected from compound (2c), compound (2d), compound (2e), compound (2f), compound (2g), compound (2h), compound (2i), compound (2j), compound (2k), compound (2l), compound (2m), compound (2n), compound (2o), compound (2p), and mixture thereof.

22. The method of any one of claims 16-21, further comprising a chemical step to form a derivative of compound (1a).

23. The method of claim 22, wherein the derivative of compound (1a) is selected from compound (1b), compound (1c), compound (1d), compound (1e), compound (1f), compound (1g), compound (1h), compound (1i), compound (1j), compound (1k), compound (1l), compound (1m), compound (1n), compound (1o), compound (1p), compound (1q), compound (1r), compound (1s), compound (1t), compound (1u), compound (1v), compound (1w), compound (1x), compound (1y), compound (1z), compound (1aa), compound (1ab), compound (1ac), compound (1ad), compound (1ae), compound (1af), compound (1ag), compound (1ah), and compound (1ai).

24. A composition comprising:
(a) a recombinant polypeptide with CYP activity comprising an amino acid sequence of at least 80% sequence identity to a sequence selected from the group consisting of SEQ ID NO: 24, 8, 10, 12, 14, 16, 18, 20, 22, 26, 28, 30, 32, 34, and 36; and
(b) a compound (2a) and/or compound (2b) or a derivative of compound (2a) and/or a derivative of compound (2b)

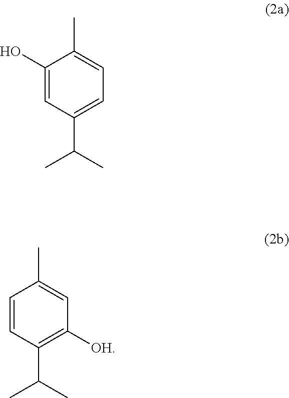

25. The composition of claim 24, further comprising a recombinant polypeptide with CPR activity; optionally, a polypeptide comprising an amino acid sequence of at least 80% sequence identity to SEQ ID NO: 6.

26. The method of any one of claims 24-25, wherein the composition comprises a derivative of compound (2a) and/or a derivative of compound (2b) selected from compound (2c), compound (2d), compound (2e), compound (2f), compound (2g), compound (2h), compound (2i), compound (2j), compound (2k), compound (2l), compound (2m), compound (2n), compound (2o), compound (2p), and mixture thereof.

27. The composition of any one of claims 24-26, wherein the composition is in aqueous solution.
